(19) United States
(12) Patent Application Publication
(10) Pub. No.: US 2012/0122679 A1
(43) Pub. Date: May 17, 2012

(54) MESOIONIC PESTICIDES

(75) Inventors: Wenming Zhang, Newark, DE (US); Caleb William Holyoke, JR., Newark, DE (US); Kenneth Andrew Hughes, Rising Sun, MD (US); George Philip Lahm, Wilmington, DE (US); Thomas Francis Pahutski, JR., Elkton, MD (US); My-Hanh Thi Tong, Bear, DE (US); Stephen Frederick McCann, Newark, DE (US)

(73) Assignee: E. I. Du Pont De Nemours and Company, Wilmington, DE (US)

(21) Appl. No.: 13/386,052

(22) PCT Filed: Aug. 3, 2010
(86) PCT No.: PCT/US2010/044264

§ 371 Date: Jan. 20, 2012

Related U.S. Application Data
(60) Provisional application No. 61/231,452, filed on Aug. 5, 2009.

(51) Int. Cl. A01N 43/90; C07D 498/04; C07D 513/04; C07D 487/04; A01N 57/28; A01C 1/06; A01N 57/16; A01N 59/14; A01N 43/68; A01N 63/00; A01N 63/04; A01P 1/00; C07D 471/04; A01N 43/22

(52) U.S. Cl. 504/100; 544/282; 514/259.4; 514/259.41; 544/278; 514/259.2; 544/280; 514/259.1; 514/120; 514/30; 514/80; 424/660; 514/245; 424/93.461; 424/93.4; 424/93.6; 424/93.5

(57) ABSTRACT

Disclosed are compounds of Formula 1, N-oxides and salts thereof,

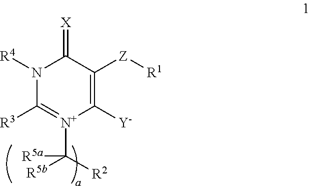

wherein
X is O or S;
Y is O or S;
Z is a direct bond, O, S(O)ₙ, NR⁶, C(R⁷)₂O, OC(R⁷)₂, EC(═X¹);
a is 1, 2 or 3;
and R¹, R², R³, R⁴, R^{5a}, R^{5b}, R⁶, R⁷, X¹ and E are as defined in the disclosure.
   Also disclosed are compositions containing the compounds of Formula 1 and methods for controlling an invertebrate pest comprising contacting the invertebrate pest or its environment with a biologically effective amount of a compound or a composition of the invention.

### FIELD OF THE INVENTION

This invention relates to certain pyrimidinium compounds, their N-oxides, salts and their compositions suitable for agronomic, nonagronomic and animal health uses, methods of their use for controlling invertebrate pests such as arthropods in both agronomic and nonagronomic environments, and for treatment of parasite infections in animals or infestations in the general environment.

### BACKGROUND OF THE INVENTION

The control of invertebrate pests is extremely important in achieving high crop efficiency. Damage by invertebrate pests to growing and stored agronomic crops can cause significant reduction in productivity and thereby result in increased costs to the consumer. The control of invertebrate pests in forestry, greenhouse crops, ornamentals, nursery crops, stored food and fiber products, livestock, household, turf, wood products, and public health is also important. Many products are commercially available for these purposes, but the need continues for new compounds that are more effective, less costly, less toxic, environmentally safer or have different sites of action.

The control of animal parasites in animal health is essential, especially in the areas of food production and companion animals. Existing methods of treatment and parasite control are being compromised due to growing resistance to many current commercial parasiticides. The discovery of more effective ways to control animal parasites is therefore imperative.

U.S. Pat. No. 5,151,427 discloses mesoionic pyrimidinium compounds of Formula i as anthelmintics

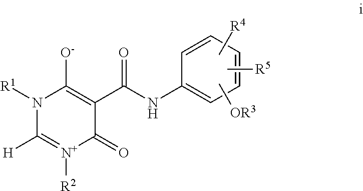

wherein, inter alia, R¹ and R² are independently C₁-C₆ alkyl, R³ is a heteroaromatic 6-membered ring, and R⁴ and R⁵ are independently hydrogen or C₁-C₄ alkyl.

The pyrimidinium compounds of the present invention are not disclosed in this publication.

### SUMMARY OF THE INVENTION

This invention is directed to compounds of Formula 1 (including all stereoisomers), N-oxides, and salts thereof, and compositions containing them and their use for controlling invertebrate pests:

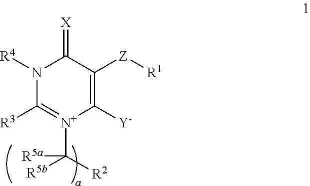

wherein
X is O or S;
Y is O or S;
Z is a direct bond, O, S(O)ₙ, NR⁶, C(R⁷)₂O, OC(R⁷)₂ or EC(═X¹);
X¹ is O, S or NR⁹;
E is O, S or NR^{9a};
R¹ is selected from the group consisting of cyano, CHO, C(═O)OH, C(═O)NH₂ and C(═S)NH₂; C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₅-C₁₀ alkylcycloalkylalkyl and C₃-C₆ cycloalkenyl, each unsubstituted or substituted with at least one substituent independently selected from R³¹; or
R¹ is a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁴;
R² is H, halogen, cyano, hydroxy, amino, nitro, —OCN, —SCN, CHO, C(═O)OH, C(═O)NH₂, C(═S)NH₂, SO₂NH₂, C(═O)R¹⁸, C(═O)OR¹⁸, NHR¹⁸, NR¹⁸R¹⁹, C(═O)NR¹⁸R¹⁹, C(═S)NR¹⁸R¹⁹, SO₂NR¹⁸R¹⁹, OC(═O)R²¹, OC(═O)OR¹⁸, OC(═O)NR¹⁸R¹⁹, N(R¹⁸)C(═O)R²¹, N(R²¹)C(═O)OR¹⁹, N(R²¹)C(═O)NR²¹R²², OSO₂R¹⁸, OSO₂NR²¹R²², NR¹⁸SO₂R¹⁸, NR²¹SO₂NR²¹R²² or Si(R¹⁸R¹⁹R²⁰); or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkotiylsulfinyl, C₂-C₈ alkenylsulfonyl, C₂-C₈ alkynylthio, C₂-C₈ alkyliylsulfinyl or C₂-C₈ alkynylsulfonyl, each unsubstituted or substituted with at least one substituent independently selected from halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ and Si(R¹⁰)₃; or a 3 to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted, with up to 5 substituents independently selected from R¹⁵;
R³ is H, C₁-C₆ alkyl, C₂-C₆ haloalkyl, C₂-C₆ alkenyl, C₂-C₆ haloalkenyl, C₂-C₆ alkynyl, C₂-C₆ haloalkynyl or C≡CR²⁵; or
   C₃-C₆ cycloalkyl or C₄-C₇ cycloalkylalkyl, each optionally substituted with up to 4 substituents independently selected from the group consisting of halogen, C₁-C₂ alkyl, 1 cyclopropyl and 1 CF₃; or
R³ is phenyl, naphthalenyi or a 5- or 6-membered heteroaromatic ring, each optionally substituted with up to 2 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino and C₂-C₆ dialkylamino;
R⁴ is C₁-C₆ alkyl, C₁-C₆ haloalkyl, C₂-C₆ alkenyl, C₂-C₆ haloalkenyl, C₂-C₆ alkynyl, C₂-C₆ haloalkynyl or C≡CR²⁵; or C₃-C₆ cycloalkyl or C₄-C₇ cycloalkylalkyl, each optionally substituted with up to 4 substituents independently selected from the group consisting of halogen, C₁-C₂ alkyl, 1 cyclopropyl and 1 CF₃; or
R⁴ is phenyl, naphthalenyi or a 5- or 6-membered heteroaromatic ring, each optionally substituted with up to 2 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ -alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino and C₂-C₆ dialkylamino; or
R³ and R⁴ are taken together with the contiguous linking nitrogen and carbon atoms to form optionally substituted ring R-1 or ring R-2

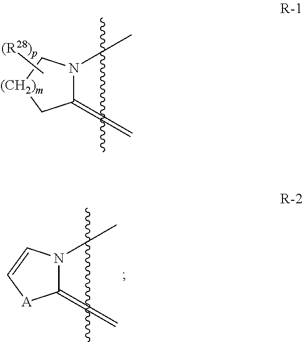

A is C(R^{29a})═(R^{29b}), S, O or NCH₃, provided that the C(R^{29a})═C(R^{29b}) moiety is oriented so the carbon atom bonded to R^{29b} is connected as R3 in Formula 1;
each R^{5a} and R^{5b} is independently H, halogen, cyano, hydroxy, amino, nitro, —OCN, —SCN, CHO, C(═O)OH, C(═O)NH₂, C(S)NH₂ or SO₂NH₂; or C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₂ cycloalkylcycloalkyl, C₅-C₈ alkylcycloalkylalkyl, C₃-C₆ cycloalkenyl, C₁-C₆ alkoxy, C₃-C₆ cycloalkoxy, C₄-C₈ cycloalkylalkoxy, C₂-C₆ alkenyloxy or C₂-C₆ alkynyloxy, each optionally substituted with halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ or Si(R¹⁰)₃;
each R⁶, R⁷ and R⁸ is independently H; or C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₆ cycloalkenyl, C₂-C₆ alkylcarbonyl or C₂-C₆ alkoxycarbonyl, each unsubstituted or substituted with at least one substituent independently selected from halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ and Si(R¹⁰)₃;
each R⁹ is independently C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₆ cycloalkenyl, C₂-C₆ alkylcarbonyl or C₂-C₆ alkoxycarbonyl, each unsubstituted or substituted with at least one substituent independently selected from halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ and Si(R¹⁰)₃;
each R^{9a} is independently H; or C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₆ cycloalkenyl, C₂-C₆ alkylcarbonyl or C₂-C₈ alkoxycarbonyl, each unsubstituted or substituted witli at least one substituent independently selected from halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ and Si(R¹⁰)₃;
each R¹⁰, R¹¹, R¹² and R¹³ is independently C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl or C₃-C₆ cycloalkenyl, each unsubstituted or substituted with at least one substituent independently selected from halogen, cyano, nitro, CHO, C(═O)OH, C(═O)NH₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl and C₃-C₆ trialkylsilyl; or phenyl or a 5- or 6-membered heteroaromatic ring, each unsubstituted or substituted with at least one substituent independently selected from C₁-C₆ alkyl, C₁-C₆ haloalkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₆ cycloalkenyl, halogen, cyano, nitro, CHO, C(═O)OH, C(═O)NH₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl and C₃-C₆ trialkylsilyl;
each R¹⁴ is independently halogen, cyano, hydroxy, amino, nitro, SF₅, —OCN, —SCN, CHO, C(═O)OH, C(═O)NH₂, C(═S)NH₂, SO₂NH2, C(═O)R¹⁸, C(═O)OR¹⁸, NHR¹⁸, NR¹⁸R¹⁹, C(═O)NR¹⁸R¹⁹, C(═S)NR¹⁸R¹⁹, SO₂NR¹⁸R¹⁹, OC(═O)R²¹, OC(═O)OR¹⁸, OC(═O)NR¹⁸R¹⁹, N(R¹⁸)C(═O)R²¹, N(R²¹)C(═O)OR¹⁹, N(R²¹)C(═O)NR²¹R²², OSO₂R¹⁸, OSO₂NR²¹R²², NR¹⁸SO₂R¹⁸, NR²¹SO₂NR²¹R²², Si(R¹⁸R¹⁹R²⁰), C(═NR²¹)R²², C(═NOR²¹)R²², C(═NNR²¹R²²)R²³, C(═NN(C(═O)R¹⁹)R²¹)R²², C(═NN(C(═O)OR¹⁹)R²¹)R²², ON═CR²¹R²², ONR²¹R²², S(═O)(═NR²¹)R²², SO₂NR²¹C(═O)NR²²R²³, P(═X²⁾R¹⁸R¹⁹, OP(═X²)R¹⁸R¹⁹, OP(═X²)(OR¹⁸)R¹⁹, OP(═X²)(OR¹⁸)OR¹⁹, N═CR²¹R²², NR²¹N═CR²²R²³, NR²¹NR²²R²³, NR²¹C(═X²)NR²²R²³, NR²¹C(═NR²¹)NR²²R²³, NR²¹NR²¹C(═X²)NR²²R²³, NR²¹NR²¹SO₂NR²²R²³, Z¹Q^{t} or Z¹QⁱQ^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₂-C₈ alkyl sulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl or C₂-C₈ alkynylsulfonyl, each unsubstituted or substituted, with at least one substituent independently selected from R¹⁷; or
two R¹⁴ substituents on adjacent ring atoms are taken together with the adjacent ring-atoms to form a 5- to 7-membered carbocyclic or heterocyclic ring, each ring containing ring members selected from carbon atoms and up to 3 heteroatoms independently selected from up to 2 O, up to 2 S, and up to 3 N, wherein up to 2-carbon atom ring members are independently selected from C(═O) and C(═S) and the sulfur atom ring members are independently selected from S(O)ₙ, each ring optionally substituted with up to 3 substituents independently selected from the group consisting of halogen, cyano, hydroxy, amino, nitro, C(═O)OH, C(═O)NH₂, SO₂NH₂, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₃-C₇ cycloalkyl, C₃-C₇ halocycloalkyl, C₄-C₈ alkylcycloalkyl, C₄-C₈ haloalkylcycloalkyl, C₄-C₈ cycloalkylalkyl, C₄-C₈ halocycloalkylalkyl, C₁-C₆ alkoxy, C₁-C₆ haloalkoxy, C₂-C₆ alkoxycarbonyl, C₂-C₆ haloalkoxycarbonyl, C₂-C₆ alkylcarbonyl and C₂-C₆ haloalkylcarbonyl;
each X² is independently O or S;
each Z¹ is independently a direct bond, O, S(O)ₙ, NR⁶, CH(R⁷), C(R⁷)═C(R⁷), C≡C, C(R⁷)₂O, OC(R⁷)₂, C(═X¹), C(═X¹)E, EC(═X¹), C(═NOR⁸) or C(═NN(R⁶)₂);
each Qⁱ is independently a 3- to 10-membered ring or a 7- to 11-membered ring-system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 4 substituents independently selected from the group consisting of halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³, Si(R¹⁰)₃ and R¹⁶;
each Q^{t} is independently a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from the group consisting of halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³, Si(R¹⁰)₃ and R¹⁶;
each R¹⁵ is independently halogen, cyano, hydroxy, amino, nitro, SF₅, —OCN, —SCN, CHO, C(═O)OH, C(═O)NH₂, C(═S)NH₂, SO₂NH2, C(═O)R¹⁸, C(═O)OR¹⁸, NHR¹⁸, NR¹⁸R¹⁹, C(═O)NR¹⁸R¹⁹, C(═S)NR¹⁸R¹⁹, SO₂NR¹⁸R¹⁹, OC(═O)R²¹, OC(═O)OR¹⁸, OC(═O)NR¹⁸R¹⁹, N(R¹⁸)C(═O)R²¹, N(R²¹)C(═O)OR¹⁹, N(R²¹)C(═O)NR²¹R²², OSO₂R¹⁸, OSO₂NR²¹R²², NR¹⁸SO₂R¹⁸, NR²¹SO₂NR²¹R²², Si(R¹⁸R¹⁹R²⁰) or Z¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkyUhio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl or C₂-C₈ alkynylsulfonyl, each unsubstituted or substituted with at least one substituent independently selected from R¹⁷; or
two R¹⁵ substituents on adjacent ring atoms are taken together with, adjacent ring atoms to form a 5- to 7-membered carbocyclic or heterocyclic ring, each ring containing ring members selected, from carbon atoms and up to 3 heteroatoms independently selected from up to 2 O, up to 2 S, and up to 3 N, wherein up to 2-carbon atom ring members are independently selected, from C(═O) and C(═S) and the sulfur atom ring members are independently selected from S(═O)ₙ, each ring optionally substituted, with up to 3 substituents independently selected from the group consisting of halogen, cyano, hydroxy, amino, nitro, C(═O)OH, C(═O)NH₂, SO₂NH₂, C₁-C₄ alkyl, C₁-C₄ haloalkyl, C₂-C₄ alkenyl, C₂-C₄ haloalkenyl, C₂-C₄ alkynyl, C₂-C₄ haloalkynyi, C₃-C₇ cycloalkyl, C₃-C₇ halocycloalkyl, C₄-C₈ alkylcycloalkyl, C₄-C₈ haloalkylcycloalkyl, C₄-C₈ cycloalkylalkyl, C₄-C₈ halocycloalkylalkyl, C₁-C₆ alkoxy, C₁-C₆ haloalkoxy, C₂-C₆ alkoxycarbonyl, C₂-C₆ haloalkoxycarbonyl, C₂-C₆ alkylcarbonyl and C₂-C₆ haloalkylcarbonyl;
each R¹⁶ is independently C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl or C₃-C₆ cycloalkenyl, each unsubstituted or substituted with at least one substituent independently selected from the group consisting of halogen, cyano, nitro, CHO, C(═O)OH, C(═O)NH₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl and C₃-C₆ trialkylsilyl; or phenyl or a 5- or 6-membered heteroaromatic ring, each unsubstituted or substituted with at least one substituent independently selected from the group consisting of C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₈ cycloalkenyl, halogen, cyano, nitro, CHO, C(═O)OH, C(═O)NH₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl and C₃-C₆ trialkylsilyl;
each R¹⁷ is independently halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³, Si(R¹⁰)₃ or Z₁Q^{t};
each R¹⁸, R¹⁹ and R²⁰ is independently C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl or C₃-C₈ cycloalkenyl, each unsubstituted or substituted, with at least one substituent independently selected, from R¹⁷; or Qₜ;
each R²¹, R²² and R²³ is independently H, C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl or C₃-C₆ cycloalkenyl, each unsubstituted or substituted with at least one substituent independently selected from R¹⁷; or Q^{t};
each R²⁴ is independently H, cyano, —OCN, —SCN, CHO, C(═O)OH, C(═O)NH₂, C(═S)NH₂, SO₂NH₂, C(═O)R¹⁸, C(═O)OR¹⁸, NHR¹⁸, NR¹⁸R¹⁹, C(═O)NR¹⁸R¹⁹, C(═S)NR¹⁸R¹⁹, SO₂NR¹⁸R¹⁹, OC(═O)R²¹, OC(═O)OR¹⁸, OC(═O)NR¹⁸R¹⁹, N(R¹⁸)C(═O)R21, N(R²¹)C(═O)OR¹⁹, N(R²¹)C(═O)NR²¹R²², OSO₂R¹⁸, OSO₂NR²¹R²², NR¹⁸SO₂R¹⁸, NR²¹SO₂NR²¹R²², Si(R¹⁸R¹⁹R²⁰) or Z¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cyeloalkylalkylsulfonyl, C₂-C₈ alkenylthio, C₂-C₈ alkeiiylsulfinyl, C₂-C₈ alkenylsulfonyl, C₂-C₈ alkynylthio, C₂-C₈ alkynylsulfinyl or C₂-C₈ alkynylsulfonyl, each unsubstituted or substituted with at least one substituent independently selected from R¹⁷;
each R²⁵ is independently Si(R³⁰)₃; or phenyl or pyridinyl, each optionally substituted with 1 to 3 substituents independently selected from the group consisting of halogen, cyano, nitro, SF₅, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₂-C₆ alkoxyalkyl, C₁-C₄ alkoxy, C₁-C₄ haloalkoxy, S(O)ₙR²⁶, S(O)₂R²⁷, C₁-C₄ alkylamino and C₂-C₆ dialkylamino;
each R²⁶ is independently C₁-C₄ alkyl or C₁-C₄ haloalkyl;
each R²⁷ is independently C₁-C₄ alkylamino, C₂-C₆ dialkylamino or —NCH₂Z²CH₂;
each Z² is independently CH₂CH₂, CH₂CH₂CH₂ or CH₂OCH₂.
each R²⁸ is independently H, halogen, cyano, CF₃, C₁-C₃ alkyl or C₃-C₆ cycloalkyl;
R^{29a} is H or F;
R^{29b} is H, F, CF₂H or CF₃;
each R³⁰ is independently C₁-C₄ alkyl;
each R³¹ is independently halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³, Si(R¹⁰)₃ or Z¹Q^{t};
a is 1, 2 or 3;
m is 0, 1, 2 or 3;
p is 0, 1, 2, 3 or 4;
each n is independently 0, 1 or 2; and
u and z in each instance of S(═O)ᵤ(═NR²⁴)_{z} are independently 0, 1 or 2, provided that the sum of u and z in each instance of S(═O)ᵤ(═NR²⁴)_{z} is 0, 1 or 2;
provided, that when
R¹ is C₁-C₆ alkyl, C₁-C₆ haloalkyl, C₂-C₆ alkenyl, C₂-C₆ haloalkenyl, C₂-C₆ alkynyl, C₂-C₆ haloalkynyl, CR³⁴═C(R³⁴)R²⁵ or C≡CR²⁵; or
   C₃-C₇ cycloalkyl, C₄-C₈ cycloalkylalkyl or C₅-C₇ cycloalkenyl, each optionally substituted with 1 to 4 substituents independently selected from the group consisting of halogen, C₁-C₂ alkyl, C₁-C₂ alkoxy, 1 cyclopropyl, 1 CF₃ and 1 OCF₃; or
   phenyl, naphthalenyl or a 5- or 6-membered heteroaromatic ring, each optionally substituted with up to 3 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ haloalkenyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxy alkyl, S(O)ₙR²⁶, S(O)₂R²⁷, C₁-C₄ alkylamino, C₂-C₆ dialkylamino, SF₅, Si(CH₃)₃, CHO, hydroxy, OC(O)R³² and N(R³³)C(O)R³²; or

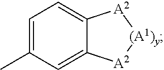

or
   CHO; or
   an 8- to 10-membered heteroaromatic bicyclic ring system optionally substituted on carbon ring members with up to 3 substituents independently selected from the group consisting of halogen, cyano, nitro, SF₅, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino, C₂-C₆ dialkylamino, Si(CH₃)₃, CHO, hydroxy, OC(O)R³² and N(R³³)C(O)R³², and optionally substituted on nitrogen ring members with methyl; or
   phenyl or a 5- or 6-membered heteroaromatic ring, each substituted with GQ¹, each optionally substituted with 1 Q² and each optionally substituted with up to 2 substituents independently selected from the group consisting of halogen, cyano, nitro, SF₅, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ -alkylamino, C₂-C₆ dialkylamino, Si(CH₃)₃, CHO, hydroxy, OC(O)R³² and N(R³³)C(O)R³²; or
   phenyl or a 5- or 6-membered heteroaromatic ring, each substituted with LQ¹ and optionally substituted with up to 2 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆, alkoxyalkyl, S(O)ₙR²⁶, S(O)₂R²⁷, C₁-C₄ alkylamino and C₂-C₅ dialkylamino;
each R³² is independently C₁-C₄ alkyl;
each R³³ is independently H or C₁-C₄ alkyl;
each R³⁴ is independently H, F or CH₃;
each A¹ is independently C(R⁵⁰)₂;
each A² is independently C(R⁵¹)₂, O, S(O)ₙ or NR⁵²;
each R⁵⁰ is independently H, F or CH₃;
each R⁵¹ is independently H or C₁-C₄ alkyl;
each R⁵² is independently C₁-C₄ alkyl;
G is a direct bond, O, S(O)ₙ, NH, N(CH₃), CH₂, CH₂O, OCH₂, C(O), C(O)O, OC(O), C(O)NH or NHC(O);
L is a phenyl or 5- or 6-membered heteroaromatic ring optionally substituted with 1 or 2 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino and C₂-C₆ dialkylamino;
Q¹ is phenyl or a 5- or 6-membered heteroaromatic ring, each optionally substituted with 1 to 3 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino, C₂-C₆ dialkylamino, SF₅, Si(CH₃)₃, CHO, hydroxy, OC(O)R³² and N(R³³)C(O)R³²;
Q² is phenyl or a 5- or 6-membered heteroaromatic ring, each optionally substituted with up to 2 substituents independently selected from the group consisting of halogen, cyano, nitro, C₁-C₄ alkyl, C₂-C₄ alkenyl, C₂-C₄ alkynyl, C₁-C₄ -haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino and C₂-C₆ dialkylamino;
a is 1;
y is 1 or 2;
R^{5a} is H, halogen, cyano or C₁-C₄ alkyl;
R^{5b} is H, halogen or CH₃;
R² is C₁-C₅ alkyl, C₁-C₅ haloalkyl, C₂-C₅ alkenyl, C₂-C₅ haloalkenyl, C₂-C₅ alkynyl, C₂-C₅ haloalkynyl, CO₂R³³, CH₂OR³³, CH₂CH₂OR³³, CH₂S(O)ₙR³³or CH₂CH₂S(O)ₙR³³; or
   C₃-C₆ cycloalkyl or C₄-C₆ cycloalkylalkyl, each optionally substituted with up to 4 substituents selected from the group consisting of halogen, C₁-C₂ alkyl, 1 cyclopropyl and 1 CF₃; or
   an optionally substituted 5- or 6-membered heterocyclic ring;
then Z is O, S(O)ₙ, NR₆, C(R⁷)₂O, OC(R⁷)₂ or EC(═X¹).

This invention is also directed to compounds selected from the group consisting of
1-[(5-chloro-2-thienyl)methyl]-2-hydrox-4-oxo-3-phenyl-4H-pyrido[1,2-a]pyrimidinium inner salt;
2-hydroxy-4-oxo-3-phenoxy 1-propyl-4H-pyrido[1,2-a]pyrimidinium inner salt;
1-[(4-chlorophenyl)methyl]-2-hydroxy-4-oxo-3-[3-(trifluoromethoxy)phenyl]-4H-pyrido[1,2-a]pyrimidinium inner salt;
1-[(6-chloro-3-pyridinyl)methyl]-2-hydroxy-4-oxo-3-[3-[[(2,2,2-trifluoroethyl)amino]carbonyl]phenyl]-4H-pyrido[1,2-a]pyrimidinium inner salt;
2-hydroxy-4-oxo-1-[2-(3-pyridinyl)ethyl]-3-[3-(trifluoromethoxy)phenyl]-4H-pyrido[1,2-a]pyrimidinium inner salt;
3-(4-fluorophenyl)-2-hydroxy-4-oxo-1-[2-(2-pyridinyl)ethyl]-4H-pyrido[1,2-a]pyrimidinium inner salt;
1-[(6-chloro-3-pyridinyl)methyl]-3-(2,3-dihydro-1H-inden-1-yl)-2-hydroxy-4-oxo-4H-pyrido[1,2-a]pyrimidinium inner salt;
1-[(2-chloro-5-thiazolyl)methyl]-3-(2,3-dihydro-1H-inden-1-yl)-2-hydroxy-4-oxo-4H-pyrido[1,2-a]pyrimidinium inner salt;
2-hydroxy-1-methyl-4-oxo-3-phenyl-4H-pyrido[1,2-a]pyrimidinium inner salt;
1-[2-(acetylamino)ethyl]-2-hydroxy-4-oxo-3-phenyl-4H-pyrido[1,2-a]pyrimidinium inner salt;
3-[3-(cyanomethoxy)phenyl]-2-hydroxy-4-oxo-1-(2,2,2-trifluoroethyl)-4H-pyrido[1,2-a]pyrimidinium inner salt;
1-[(6-chloro-3-pyridinyl)methyl]-3-[3-(cyanomethoxy)phenyl]-2-hydroxy-4-oxo-4-H-pyrido[1,2-a]pyrimidinium inner salt;
3-[2-[(6-chloro-3-pyridinyl)methoxy]-4-fluorophenyl]-2-hydroxy-4-oxo-1-(2,2,2-trifluoroethyl)-4H-pyrido[1,2-a]pyrimidinium inner salt;
3-[3-[(6-chloro-3-pyridinyl)methoxy]phenyl]-2-hydroxy-4-oxo-1-(2,2,2-trifluoroethyl)-4H-pyrido[1,2-a]pyrimidinium inner salt;
3-[3-[(6-chloro-3-pyridinyl)methoxy]phenyl]-1-[(6-chloro-3-pyridinyl)methyl]2-hydroxy-4-oxo-4H-pyrido[1,2-a]pyrimidinium inner salt;
1-[(6-chloro-3-pyridinyl)methyl]-2-hydroxy-4-oxo-3-[3-(2,2,2-trifluoroethoxy)phenyl]-4H-pyrido[1,2-a]pyrimidinium inner salt;
1-[(6-chloro-3-pyridinyl)methyl]-3-[4-fluoro-3-(2-pyridinylmethoxy)phenyl]-2-hydroxy-4-oxo-4H-pyrido[1,2-o]pyrimidinium inner salt;
3-[3-[(6-chloro-2-pyridinyl)methoxy]phenyl]-2-hydroxy-4-oxo-1-(2,2,2-trifluoroethyl)-4H-pyrido[1,2-a]pyrimidinium inner salt;
2-hydroxy-1-[(4-methoxyphenyl)methyl]-4-oxo-3-phenyl-4H-pyrido[1,2-a]pyrimidinium inner salt;
3-[3-(3-bromo-4,5-dihydro-5-isoxazolyl)phenyl]-1-[(2-chloro-5-thiazolyl)methyl]-2-hydroxy-4-oxo-4H-pyrido[1,2-a]pyrimidinium inner salt;
1-[(2-chloro-5-thiazolyl)methyl]-3-[3-(4,5-dihydro-3-methyl-5-isoxazolyl)phenyl]2-hydroxy-4-oxo-4H-pyrido[1,2-a]pyrimidinium inner salt;
1-(1,3-dioxolan-2-ylmethyl)-2-hydroxy-4-oxo-3-phenyl-4H-pyrido[1,2-a]pyrimidinium inner salt;
1-(1,4-dioxolan-2-ylmethyl)-2-hydroxy-4-oxo-3-phenyl-4H-pyrido[1,2-o]pyrimidinium inner salt; and
1-[(2-chloro-5-thiazolyl)methyl]-2-hydroxy-3-[3-[1-(methoxyimino)ethyl]phenyl]-4-oxo-4H-pyrido[1,2-a]pyrimidinium inner salt,
   to their compositions comprising at least one additional component selected from the group consisting of surfactants, solid diluents and liquid diluents, said compositions optionally further comprising at least one additional biologically active compound or agent, and to their methods of use for controlling an invertebrate pest comprising contacting the invertebrate pest or its environment with a biologically effective amount of said compound (e.g., as a composition described herein).

This invention also provides a composition comprising a compound of Formula 1, an N-oxide or a salt thereof, and at least one additional component selected from the group consisting of surfactants, solid diluents and liquid diluents. In one embodiment, this invention also provides a composition for controlling an invertebrate pest comprising a compound of Formula 1, an N-oxide, or a salt thereof, and at least one additional component selected from the group consisting of surfactants, solid diluents and liquid diluents, said composition further comprising at least one additional biologically active compound or agent.

This invention further provides a composition for protecting an animal from an invertebrate parasitic pest comprising a parasiticidally effective amount of a compound of Formula 1, an N-oxide, or a salt thereof, and at least one earner.

This invention further provides a spray composition for controlling an invertebrate pest comprising a compound of Formula 1, an N-oxide, or a salt thereof, or the compositions described above, and a propellant. This invention also provides a bait composition for controlling an invertebrate pest comprising a compound of Formula 1, an N-oxide, or a salt thereof, or the compositions described, in the embodiments above, one or more food, materials, optionally an attractant, and optionally a humectant.

This invention further provides a trap device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted, to be placed in or near a locus of potential or known activity for the invertebrate pest.

This invention provides a method for controlling an invertebrate pest comprising contacting the invertebrate pest or its environment with a biologically effective amount of a compound of Formula 1, an N-oxide, or a salt thereof (e.g., as a composition described herein). This invention also relates to such method wherein the invertebrate pest or its environment is contacted with a composition comprising a biologically effective amount of a compound of Formula 1, an N-oxide, or a salt thereof, and at least one additional component selected, from the group consisting of surfactants, solid diluents and liquid diluents, said composition optionally further comprising a biologically effective amount of at least one additional biologically active compound or agent.

This invention also provides a method for controlling an invertebrate pest comprising contacting the invertebrate pest or its environment with a biologically effective amount of any of the aforesaid compositions wherein the environment is a plant.

This invention also provides a method, for controlling an invertebrate pest comprising contacting the invertebrate pest or its environment with a biologically effective amount of any of the aforesaid compositions wherein the environment is an animal.

This invention also provides a method for controlling an invertebrate pest comprising contacting the invertebrate pest or its environment with a biologically effective amount of any of the aforesaid compositions wherein the environment is a seed.

This invention also provides a method for protecting a seed from an invertebrate pest comprising contacting the seed with a biologically effective amount of a compound of Formula 1, an N-oxide, or a salt thereof (e.g., as a composition described herein). This invention also relates to the treated seed.

This invention further provides a method for treating, preventing, inhibiting and/or killing ecto and/or endoparasites comprising administering to and/or on an animal a parasiticidaily effective amount of a compound of Formula 1, an N-oxide, or a salt thereof (e.g., as a composition described herein). This invention also relates to such method wherein a parasiticidally effective amount of a compound of Formula 1, an N-oxide, or a salt thereof, (e.g., as a composition described herein) is administered to an environment (e.g., a stall or blanket) in which an animal resides.

### DETAILS OF THE INVENTION

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method, that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause: other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods and nematodes of economic importance as pests. The term “arthropod” includes insects, mites, spiders, scorpions, centipedes, millipedes, pill bugs and symphylans. The term “gastropod” includes snails, slugs and other Stylommatophora. The term “nematode” refers to a living organism of the Phylum Nematoda. The term “helminths” includes roundworms, heartworms, and phytophagous nematodes (Nematoda), flukes (Tematoda), Acanthocephala, and tapeworms (Cestoda).

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of corn, soybeans and other legumes, rice, cereal (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

Nonagronomic applications include protecting an animal from an invertebrate parasitic pest by administering a parasiticidally effective (i.e. biologically effective) amount of a compound of the invention, typically in the form of a composition formulated, for veterinary use, to the animal to be protected. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished, mobility or lessened, ability to remain on or in the host animal, reduced feeding and inhibition of reproduction. These effects on invertebrate parasite pests provide control (including prevention, reduction or elimination) of parasitic infestation or infection of the animal.

In the above recitations, the term “alkyl”, used either alone or in compound words such as “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopenrylethyl, and other cycloalkyl moieties bonded, to straight-chain or branched alkyl groups. “Cycloalkenyl” includes groups such as cyclopentenyl and cyclohexenyl as well as groups with more than one double bond such as 1,3- and 1,4-cyclohexadienyl. The term “cycloalkoxy” denotes cycloalkyl attached, to and linked through an oxygen atom such as cyclopentyloxy and cyclohexyloxy. “Alkylcycloalkylalkyl” denotes an alkyl group substituted with alkylcycloalkyl. Examples of “alkylcycloalkylalkyl” include 1-, 2-, 3- or 4-methyl or -ethyl cyclohexylmethyl. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyelopropyl (such as 1,1′-bicyclopropyl-1-yl, 1,1′-bicyclopropyl-2-yl), cyclohexyl cyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl). “Cycloalkylamino” denotes an NH radical substituted with cycloalkyl. Examples of “cycloalkylamino” include cyclopropylamino and cyclohexylamino. The term “cycloalkylaminoalkyl” denotes cycloalkylamino substitution on an alkyl group. Examples of “cycloalkylaminoalkyl” include cyclopropylaminomethyl, cyclopentylaminoethyl, and other cycloalkylamino moieties bonded to straight-chain or branched alkyl groups.

The term “halogen”, either alone or in compound words such as “haloalkyl”, or when used in descriptions such as “alkyl substituted with halogen” includes fluorine, chlorine, bromine or iodine. Further, when used in compound, words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include CF₃, CH₂Cl, CH₂CF₃ and CCl₂CF₃. The terms “haloalkenyl”, “haloalkynyl” “haloalkoxy”, “haloalkylthio”, “haloalkylamino”, “haloalkylsulfinyl”, “haloalkylsulfonyl”, “halocycloalkyl”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylamino” include CF₃(CH₃)CHNH, (CF₃)₂CHNH and CH₂ClCH₂NH. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S(═O)₂. Examples of “halocycloalkyl” include 2-chlorocyclopropyl, 2-fluorocyclobutyl, 3-bromocyclopentyl and 4-chlorocyclohexyl. The term “halodialkyl”, either alone or in compound words such as “halodialkylamino”, means at least one of the two alkyl groups is substituted with at least one halogen atom, and independently each halogenated alkyl group may be partially or fully substituted, with halogen atoms which may be the same or different. Examples of “halodialkylamino” include (BrCH₂CH₂)₂N and BrCH₂CH₂(ClCH₂CH₂)N.

“Alkoxy” includes, for example, methoxy, ethoxy, n-propoxy, isopropoxy and the different butoxy, pentoxy and hexyloxy isomers. “Alkoxyalkyl” denotes alkoxy substitution on alkyl. Examples of “alkoxyalkyl” include CH₂OCH₃, CH₂CH₂OCH₃, CH₂OCH₂CH₃, CH₂OCH₂CH₂CH₂CH₃ and CH₂CH₂OCH₂CH₃. “Alkenyloxy” includes straight-chain or branched alkenyl attached, to and linked through an oxygen atom, Examples of “alkenyloxy” include H₂C═CHCH₂O, (CH₃)₂C≡CHCH₂O, (CH₃)CHCH₂O, (CH₃)CH═C(CH₃)CH₂O and CH₂═CHCH₂CH₂O. “Alkynyloxy” includes straight-chain or branched alkynyloxy moieties. Examples of “alkynyloxy” include HC≡CCH₂O, CH₃C≡CCH₂O and CH₃C≡CCH₂CH₂O.

The term “alkylthio” includes straight-chain or branched alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O) and the different butylsulfinyl, pentylsulfinyl and hexylsulfinyl isomers. Examples of “alkylsulfonyl” include CH₃S(═O)₂, CH₃CH₂S(═O)₂, CH₃CH₂CH₂S(═O)₂, (CH₃)₂CHS(═O)₂, and the different butylsulfonyl, pentylsulfonyl and hexylsulfonyl isomers.

“Alkylamino” denotes an NH radical substituted with straight-chain or branched alkyl. Examples of “alkylamino” include NHCH₂CH₃, NHCH₂CH₂CH₃, and NHCH₂CH(CH₃)₂. “Dialkylamino” denotes an N radical substituted independently with two straight-chain or branched alkyl groups. Examples of “dialkylamino” include N(CH₃)₂, N(CH₃CH₂CH₂)₂ and N(CH₃)CH₂CH₃. “Halodialkylamino” denotes one straight-chain or branched alkyl moiety and one straight-chain or branched haloalkyl moiety bonded to an N radical, or two independent straight-chain or branched haloalkyl moieties bonded to an N radical, wherein “haloalkyl” is as defined above. Examples of “halodialkylamino” include N(CH₂CH₃)(CH₂CH₂Cl) and N(CF₂CF₃)₂.

“Alkylcarbonyl” denotes a straight-chain or branched alkyl moiety bonded to a C(O) moiety. The chemical abbreviations C(O) and C(═O) as used herein represent a carbonyl moiety. Examples of “alkylcarbonyl” include C(O)CH₃, C(O)CH₂CH₂CH₃ and C(O)CH(CH₃)₂. Examples of “haloalkylcarbonyl” include C(O)CF₃, C(O)CCl₃, C(O)CH₂CF₃ and C(O)CF₂CF₃.

“Alkoxycarbonyl” denotes a straight-chain or branched alkyl moiety bonded to a CO₂ moiety. The chemical abbreviations CO₂ and C(O)O as used herein represent an ester moiety. Examples of “alkoxycarbonyl” include C(O)OCH₃, C(O)OCH₂CH₃, C(O)OCH₂CH₂CH₃ and C(O)OCH(CH₃)₂.

“Alkylaminocarbonyl” denotes a straight-chain or branched, alkyl moiety bonded to a C(O)NH moiety. The chemical abbreviations C(O)NH, and C(O)N as used herein represent an amide moiety (i.e. an aminocarbonyl group). Examples of “alkylaminocarbonyl” include C(O)NHCH₃, C(O)NHCH₂CH₂CH₃ and C(O)NHCH(CH₃)₂. “Dialkylaminocarbonyl” denotes two independent straight-chain or branched alkyl moieties bonded to a C(O)N moiety. Examples of “dialkylaminocarbonyl” include C(O)N(CH₃)₂ and C(O)N(CH₃)(CH₂CH₃).

“Trialkylsilyl” includes 3 branched and/or straight-chain alkyl radicals attached to and linked through a silicon atom, such as trimethylsilyl, triethylsilyl and tert-butyldimethylsilyl.

“CHO” means formyl, “OCN” means —O—C≡N, and “SCN” means —S—C≡N.

When A is C(R^{29a})═C(R^{29b}), the C(R^{29a})═C(R^{29b}) moiety is oriented so the carbon atom bonded to R^{29b} is connected directly to the pyrimidinium ring of Formula 1 as shown below.

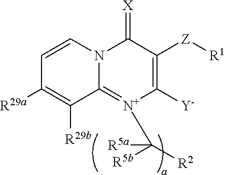

When Z is EC(═X¹), the moiety is oriented so the E atom is connected directly to the pyrimidinium ring of Formula 1 and the C(═X¹) function is connected to R¹.

The total number of carbon atoms in a substituent group is indicated by the “Cᵢ-Cⱼ” prefix where i and j are numbers from 1 to 14. For example, C₁-C₄ alkyl designates methyl through butyl; C₂ alkoxyalkyl designates CH₂OCH₃: C₃ alkoxyalkyl designates, for example, CH₃CH(OCH₃), CH₂CH₂OCH₃ or CH₂OCH₂CH₃; and C₄ alkoxyalkyl designates the various isomers of an alkyl group substituted with an alkoxy group containing a total of four carbon atoms, examples including CH₂OCH₂CH₂CH₃ and CH₂CH₂OCH₂CH₃.

When a group contains a substituent which can be hydrogen, for example R², then when this substituent is taken as hydrogen, it is recognized that this is equivalent to said group being unsubstituted. When a variable group is shown to be optionally attached to a position, for example (R^{v})ᵣ in U-36 of Exhibit 1 wherein r may be 0, then hydrogen can be at the position even if not recited in the variable group definition. When one or more positions on a group are said to be “not substituted” or “unsubstituted”, then hydrogen atoms are attached to take up any free valency.

Unless otherwise indicated, a “ring” or “ring system” as a component of Formula 1 (e.g., R¹ is a 3- to 10-membered ring or a 7- to 11-membered ring system) is carbocyclic or heterocyclic. The term “ring system” denotes two or more connected rings. The term “bicyclic ring system” denotes a ring system consisting of two rings sharing two or more common atoms.

A ring or a bicyclic ring system can be part of an extended ring system containing more than two rings wherein substituents on the ring or bicyclic ring system are taken together to form the additional rings, which may be in bicyclic relationships with other rings in the extended ring system.

The term “ring member” refers to an atom (e.g., C, O, N or S) or other moiety (e.g., C(═O), C(═S) or S(═O)ᵤ(═NR²⁴)_{z}) forming the backbone of a ring or ring system. The term “aromatic” indicates that each of the ring atoms is essentially in the same plane and has ap-orbital perpendicular to the ring plane, and that (4n+2) π electrons, where n is a positive integer, are associated with the ring or ring system to comply with Huckel's rule.

“Partially saturated” and “partially unsaturated” with reference to a ring or ring system means that the ring or ring system contains at least one double bond, but the ring or ring system is not aromatic. A ring system is aromatic if at least one component ring is aromatic.

The term “carbocyclic ring” denotes a ring wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

The terms “heterocyclic ring” or “heterocycle” denotes a ring wherein at least one of the atoms forming the ring backbone is other than carbon. Unless otherwise indicated, a heterocyclic ring can be a saturated, partially unsaturated, or fully unsaturated ring. “Saturated heterocyclic ring” refers to a heterocyclic ring containing only single bonds between ring members. “Partially saturated heterocyclic ring” refers a heterocyclic ring containing at least one double bond but which is not aromatic. The term “heteroaromatic ring” denotes a fully unsaturated aromatic ring in which at least one atom forming the ring backbone is not carbon. Typically a heteroaromatic ring contains no more than 4 nitrogens, no more than 1 oxygen and no more than 1 sulfur. Unless otherwise indicated, heteroaromatic rings can be attached through any available carbon or nitrogen by replacement of a hydrogen on said carbon or nitrogen. The term “heteroaromatic bicyclic ring system” denotes a ring system consisting of two fused rings, in which at least one of the two rings is a heteroaromatic ring as defined above.

When a radical (e.g., a 3- to 10-nienibered ring or a 7- to 11-membered ring system in the definition of R¹) is optionally substituted with the number of substituents stated (e.g., “up to 5”), then the radical may be unsubstituted or substituted with a number of substituents ranging up to the high number stated (e.g., “5”), and the attached substituents are independently selected from the substituents listed.

When a substituent (e.g., R¹) is a ring or ring system, it can be attached to the remainder of Formula 1 through any available ring member, unless otherwise described.

As noted above, R¹ is, inter alia, a 3- to 10-membered ring or a 7- to 11-membered ring system, each ring or ring system containing ring members selected from carbon atoms and up to 4 heteroatoms independently selected, from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)₂, each ring or ring system optionally substituted with up to 5 substituents independently selected, from R¹⁴ and R³². In this definition the ring members selected from up to 2 O, up to 2 S and up to 4 N are optional, because the number of heteroatom ring members may be zero. When no heteroatom ring members are present, the ring or ring system is carbocyclic. If at least one heteroatom ring member is present, the ring or ring system is heterocyclic. The definition of S(═O)ᵤ(═NR²⁴)_{z} allows up to 2 sulfur ring members, which can be oxidized sulfur moieties (e.g., S(═O) or S(═O)₂) or unoxidized sulfur atoms (i.e. when u and z are both zero). The nitrogen atom ring members may be oxidized as N-oxides, because compounds relating to Formula 1 also include N-oxide derivatives. The up to 3 carbon atom ring members selected from C(═O) and C(═S) are in addition to the up to 4 heteroatoms selected from up to 2 O, up to 2 S and up to 4 N atoms. As the R¹⁴ and R³² substituents are optional when a is 2 or 3, 0 to 5 substituents may be present, limited, only by the number of available points of attachment. When a is 1 at least one R³² substituent must be present.

The term “unsubstituted” in connection with, a group such as a ring or ring system means the group does not have any substituents other than its one or more attachments to the remainder of Formula 1. The term “optionally substituted” in connection with a group such as a ring or ring system (e.g., 5-membered heterocyclic ring of R¹) without specifying the number or identity of optional substituents refers to groups that are unsubstituted or have at least one non-hydrogen substituent that does not extinguish insecticidal activity of the unsubstituted analog. The term “optionally substituted” means that the number of substituents can be zero. Unless otherwise indicated, optionally substituted groups may be substituted, with as many optional substituents as can be accommodated by replacing a hydrogen atom with a non-hydrogen substituent on any available carbon or nitrogen atom. Commonly, the number of optional substituents (when present) ranges from 1 to 3.

The number of optional substituents may be restricted by an expressed limitation. For example, the phrase “optionally substituted with up to 5 substituents independently selected from R¹⁴” means that 0, 1, 2, 3, 4 or 5 substituents can be present (if the number of potential connection points allows). When a range specified for the number of substituents exceeds the number of positions available for substituents on a ring, the actual higher end of the range is recognized to be the number of available positions.

When the number of optional substituents is not restricted by an expressed limitation (e.g., the phrases “optionally substituted with halogen” or “unsubstituted or substituted with at least one substituent independently selected, from”), it is understood to mean that the number of optional substituents can range from 0 up to the number of positions available. One skilled in the art will appreciate that while some substituents such as halogen can be present at every available position (for example, the C₂F₅ substituent is a C₂ alkyl group substituted with the maximum number of 5 fluorine atoms), practical factors such as cost and synthetic accessibility can limit the number of occurences of other substituents. These limitations are part of the general synthetic knowledge known to those skilled, in the art. Of note are embodiments wherein in the absence of expressed limitation of number of optional substituents, the number of optional substituents is up to 3 (i.e. 0, 1, 2 or 3) if accommodated by the number of available positions.

As noted above, substituents such as R¹ can be (among others) a 5- or 6-membered heteroaromatic ring, optionally substituted with one or more substituents selected, from a group of substituents as defined in the Summary of Invention. Examples of a 5- or 6-membered heteroaromatic ring optionally substituted with one or more substituents include the rings U-2 through U-61 illustrated in Exhibit 1 wherein R^{v} is any substituent as defined in the Summary of the Invention (e.g., for R¹) and r is an integer from 0 to 2, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{v})ᵣ.

### Exhibit 1

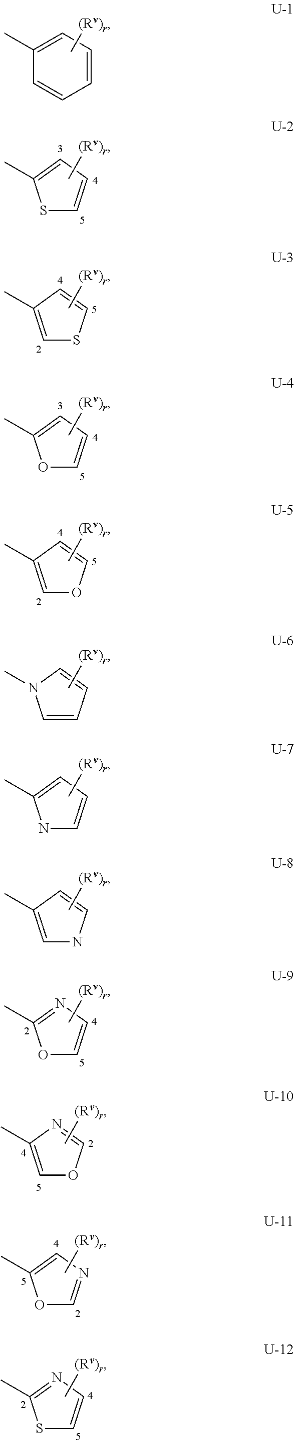

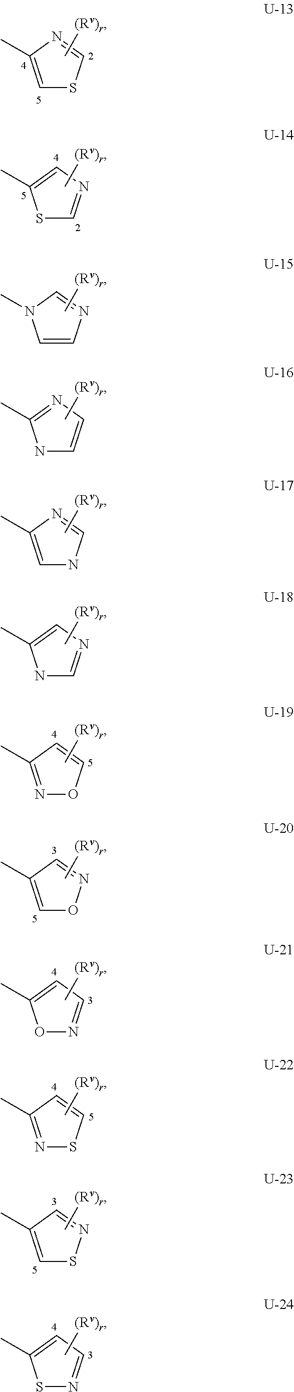

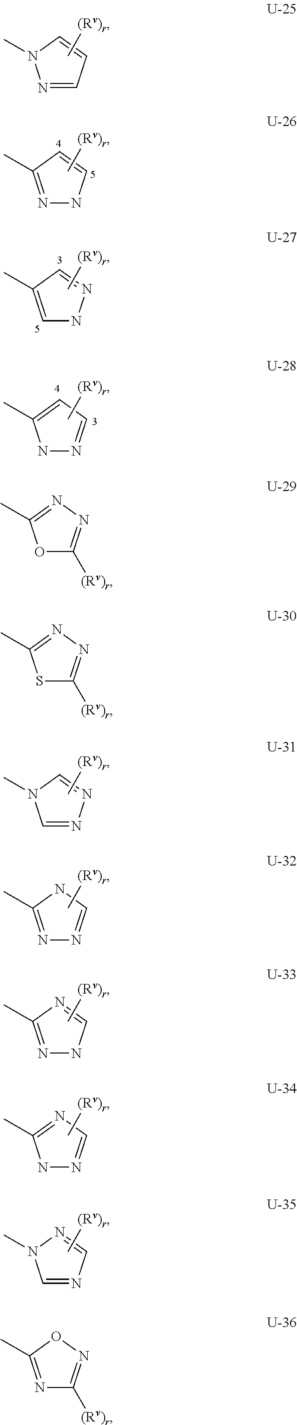

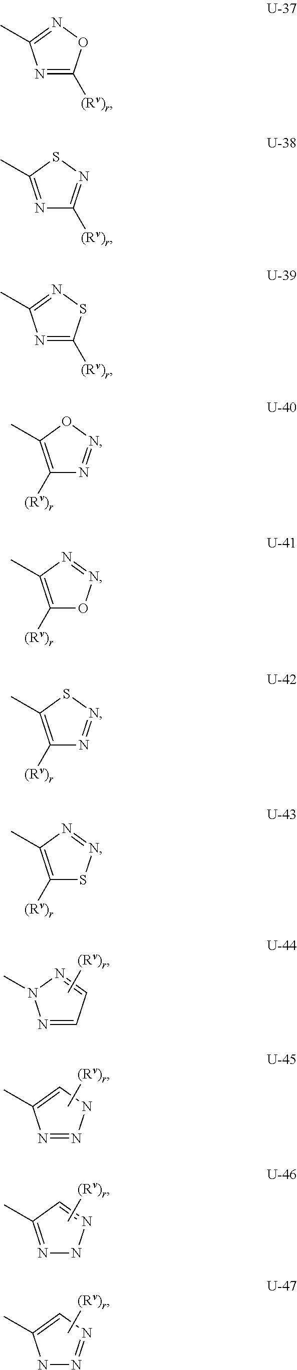

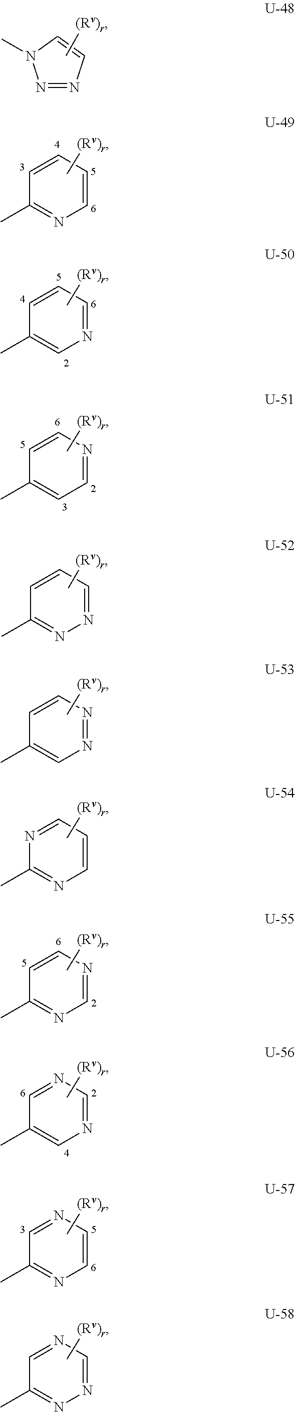

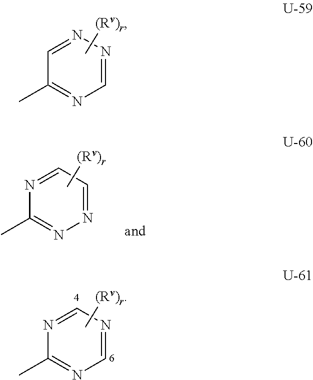

As noted above, substituents such as R¹ can be (among others) an 8-, 9- or 10-membered heteroaromatic bicyclic ring system optionally substituted, with up to 5 substituents selected from a group of substituents as defined, in the Summary of Invention. Examples of a 8-, 9- or 10-membered heteroaromatic bicyclic ring system optionally substituted, with up to 5 substituents include the ring systems H-1 through H-23 illustrated in Exhibit 2 wherein R^{v} is any substituent as defined in the Summary of the Invention (e.g., for R¹) and r is an integer from 0 to 5, limited by the number of available positions on each H group.

### Exhibit 2

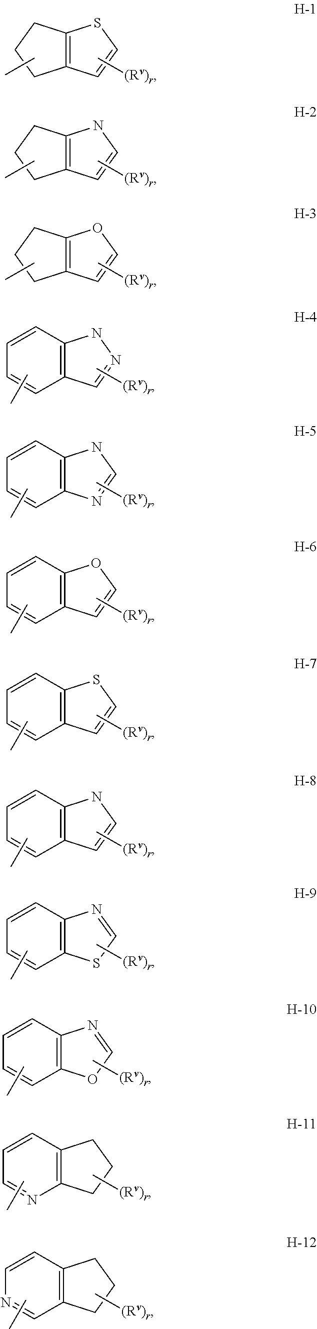

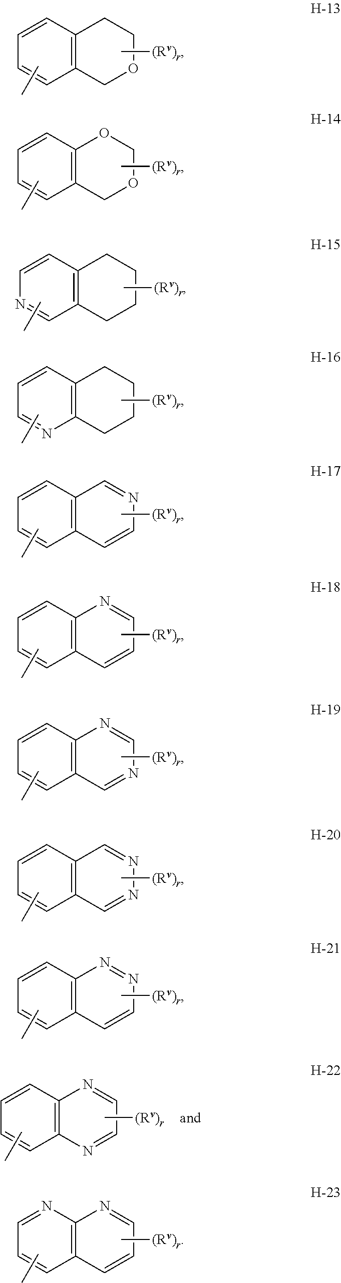

Although R^{v} groups are shown in the structures U-1 through U-61 and H-1 through H-23, it is noted that they do not need to be present since they are optional substituents. The nitrogen atoms that require substitution to fill their valence are substituted with H or R^{v}. Note that when the attachment point between (R^{v})ᵣ and the U or H group is illustrated as floating, (R^{v})ᵣ can be attached to any available carbon atom or nitrogen atom of the U or H group. Note that when the attachment point on the U or H group is illustrated as floating, the U or H group can be attached to the remainder of Formula 1 through any available carbon or nitrogen of the U or H group by replacement of a hydrogen atom. Note that some U groups can only be substituted with less than 2 R^{v} groups (e.g., U-2 through U-5, U-7 through U-48, and U-52 through U-61).

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A, R. Katritzky, C, W. Rees and E. F. V. Scriven editors-in-chief, Pergamon Press, Oxford, 1996.

The compounds of Formula 1 are mesoionic inner salts. “Inner salts”, also known in the art as “zwitterions”, are electrically neutral molecules but cany formal positive and negative charges on different atoms in each valence bond structure according to valence bond theory. Furthermore the molecular structure of the compounds of Formula 1 can be represented by the six valence bond structures shown below, each placing the formal positive and negative charges on different atoms. Because of this resonance, the compounds of Formula 1 are also described as “mesoionic”. Although for sake of simplicity, the molecular structure of Formula 1 is depicted as a single valence bond structure herein, this particular valence bond structure is to be understood as representative of all six valence bond structures relevant to bonding in molecules of compounds of Formula 1. Therefore reference to Formula 1 herein relates to all six applicable valence bond structures and other (e.g., molecular orbital theory) structures unless otherwise specified.

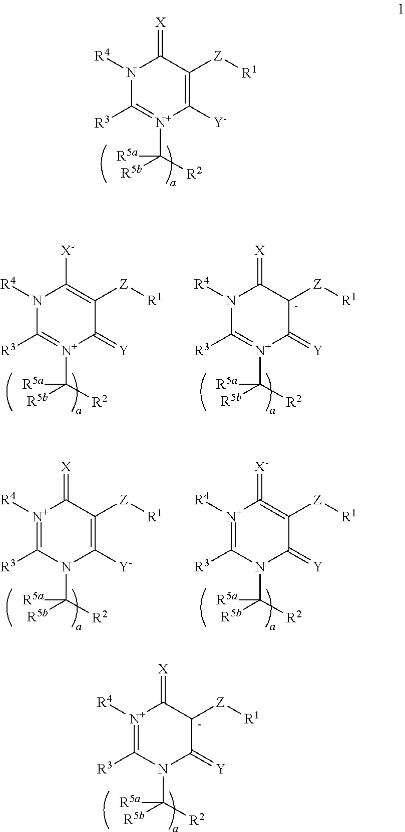

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

Compounds of this invention can exist as one or more conformational isomers due to restricted bond rotation caused by steric hinderance. For example, a compound of Formula 1 wherein Z is a direct bond and R¹ is phenyl substituted in the ortho-position with a sterically demanding alkyl group (e.g., isopropyl) may exist as two retainers due to restricted rotation about the R¹-pyrimidinium ring bond. This invention comprises mixtures of conformational isomers. In addition, this invention includes compounds that are enriched in one conformer relative to others.

Compounds selected from Formula 1 (including all stereoisomers, N-oxides, and salts thereof) typically exist in more than one form, and Formula 1 thus includes ail crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound, that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

One skilled in the art will appreciate that not all nitrogen-containing heterocyclics can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled, in the art including the oxidation of heterocycles and tertiary amines with peroxy acids such as peracetic and 3-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds. Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of invertebrate pests and animal parasites (i.e. are suitable for animal health use). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toiuenesulfonic or valeric acids. When a compound, of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and salts thereof.

Embodiments of the present invention as described in the Summary of the Invention include those described below. In the following Embodiments Formula 1 includes stereoisomers, N-oxides, and salts thereof, and reference to “a compound of Formula 1” includes the definitions of substituents specified in the Summary of the Invention unless further defined in the Embodiments.
Embodiment 1. A compound of Formula 1 wherein X is O.
Embodiment 2. A compound of Formula 1 wherein X is S.
Embodiment 3. A compound of Formula 1 or Embodiments 1 or 2 wherein Y is O.
Embodiment 4. A compound of Formula 1 or Embodiments 1 or 2 wherein Y is S.
Embodiment 5. A compound of Formula 1 or any one of Embodiments 1-4 wherein when R³ is taken alone (i.e. not taken together with R4 to form an optionally substituted ring), then R³ is H, C₁-C₆ alkyl, C₁-C₆ haloalkyl, C₂-C₆ alkenyl, C₂-C₆ haloalkenyl, C₂-C₆ alkynyl, C₂-C₆ haloalkynyl or C≡CR²⁵; or C₃-C₆ cycloalkyl or C₄-C₇ cycloalkylalkyl, each optionally substituted with up to 4 substituents independently selected from the group consisting of halogen, C₁-C₂ alkyl, 1 cyclopropyl and 1 CF₃.
Embodiment 5a. A compound of Embodiment 5 wherein when R³ is taken alone, then R³ is C₁-C₆ alkyl, C₁-C₆ haloalkyl or C₃-C₆ cycloalkyl.
Embodiment 5b. A compound of Embodiment 5 wherein when R³ is taken alone, then R³ is C₁-C₆ alkyl, C₁-C₆ haloalkyl or cyclopropyl.
Embodiment 5c. A compound of Embodiment 5b wherein when R³ is taken alone, then R³ is CH₃, CH₂CH₃ or cyclopropyl.
Embodiment 5d. A compound of Embodiment 5a wherein when R³ is taken alone, then R³ is C₃-C₆ cycloalkyl.
Embodiment 6. A compound of Formula 1 or any one of Embodiments 1-5d wherein when R⁴ is taken alone (i.e. not taken together with R³ to form an optionally substituted ring), then R⁴ is C₁-C₆ alkyl, C₁-C₆ haloalkyl, C₂-C₆ alkenyl, C₂-C₆ haloalkenyl, C₂-C₆ alkynyl, C₂-C₆ haloalkynyl or C≡CR²⁵; or C₃-C₆ cycloalkyl or C₄-C₇ cycloalkylalkyl, each optionally substituted with up to 4 substituents independently selected from the group consisting of halogen, C₁-C₂ alkyl, 1 cyclopropyl and 1 CF₃.
Embodiment 6a. A compound of Embodiment 6 wherein when R⁴ is taken alone, then R⁴ is C₁-C₆ alkyl, C₁-C₆ haloalkyl or C₃-C₆ cycloalkyl.
Embodiment 6. A compound of Embodiment 6 wherein when R⁴ is taken alone, then R⁴ is C₁-C₆ alkyl, C₁-C₆ haloalkyl or C₃-C₆ cyclopropyl.
Embodiment 6c. A compound of Embodiment 6 wherein when R⁴ is taken alone, then R⁴ is C₃-C₆ cycloalkyl.
Embodiment 6d. A compound of Embodiment 6b wherein when R⁴ is taken alone, then R⁴ is CH₃.
Embodiment 6e. A compound of Formula 1 or any one of Embodiments 1-6d wherein R³ and R⁴ are each taken alone.
Embodiment 7. A compound of Formula 1 or any one of Embodiments 1-4 wherein R³ and R⁴ are taken together with the contiguous linking nitrogen and carbon atoms to form optionally substituted, ring R-1 or R-2.
Embodiment 7a. A compound of Formula 1 or any one of Embodiments 1-7 wherein when R³ and R⁴ are taken together with the contiguous linking nitrogen and carbon atoms to form an optionally substituted ring, then the ring is R-1.
Embodiment 7b. A compound of Formula 1 or anyone of Embodiments 1-7 wherein when R³ and R⁴ are taken together with the contiguous linking nitrogen and carbon atoms to form an optionally substituted ring, then the ring is R-2.
Embodiment 8. A compound of Formula 1 or any one of Embodiments 1-7b wherein m is 2 or 3.
Embodiment 8a. A compound of Embodiment 8 wherein m is 2.
Embodiment 9. A compound of Formula 1 or any one of Embodiments 1-8a wherein p is 0 or 1.
Embodiment 9a. A compound of Embodiment 9 wherein p is 1.
Embodiment 10. A compound of Formula 1 or any one of Embodiments 1-9a wherein R²⁸ is CH₃.
Embodiment 11. A compound of Formula 1 or any one of Embodiments 1-10 wherein A is C(R^{29a})═C(R^{29b}), S or NCH₃ (provided that the C(R^{29a})═C(R^{29b}) moiety is oriented so the carbon atom bonded to R^{29b} is connected as R³ in Formula 1).
Embodiment 11a. A compound of Embodiment 11 wherein A is C(R^{29a})═C(R^{29b}) or NCH₃.
Embodiment 11b. A compound of Embodiment 11a wherein A is C(R^{29a})═C(R^{29b}).
Embodiment 11c. A compound of Embodiment 11a wherein A is NCH₃.
Embodiment 11d. A compound of Embodiment 11 wherein A is S.
Embodiment 12. A compound of Formula 1 or any one of Embodiments 1-11c wherein Z is a direct bond, O or NR⁶.
Embodiment 12a. A compound of Embodiment 12 wherein Z is NR⁶.
Embodiment 12b. A compound of Embodiment 12 wherein Z is O.
Embodiment 12c. A compound of Embodiment 12 wherein Z is a direct bond.
Embodiment 13. A compound of Formula 1 or any one of Embodiments 1-12c wherein X¹ is O, S or NR⁹.
Embodiment 13a. A compound of Embodiment 13 wherein X¹ is O.
Embodiment 13b. A compound of Embodiment 13 wherein X¹ is S.
Embodiment 14. A compound of Formula 1 or any one of Embodiments 1-13b wherein E is O.
Embodiment 14a. A compound of Formula 1 or any one of Embodiments 1-13b wherein E is S.
Embodiment 14b. A compound of Formula 1 or any one of Embodiments 1-13b wherein E is NR^{9a}.
Embodiment 15. A compound of Formula 1 or any one of Embodiments 1-14b wherein each R9 is independently C₁-C₆ alkyl, C₂-C₆ alkylcarbonyl or C₂-C₆ alkoxycarbonyl.
Embodiment 15a. A compound of Embodiment 15 wherein each R₉ is independently CH₃, C(═O)CH₃ or C(═O)OCH₃.
Embodiment 16. A compound of Formula 1 or any one of Embodiments 1-15a wherein each R^{9a} is independently H, C₁-C₆ alkyl, C₂-C₆ alkylcarbonyl or C₂-C₆ alkoxycarbonyl.
Embodiment 16a. A compound of Embodiment 16 wherein each R^{9a} is independently H, CH₃, C(═O)CH₃ or C(═O)OCH₃.
Embodiment 17. A compound of Formula 1 or any one of Embodiments 1-16a wherein R¹ is C₁-C₈ alkyl unsubstituted or substituted, with at least one substituent independently selected from R³¹; or a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁴,
Embodiment 17a. A compound of Embodiment 17 wherein R¹ is C₁-C₈ alkyl unsubstituted or substituted with at least one substituent independently selected from the group consisting of halogen and Z¹Q^{t}; or a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁴.
Embodiment 17b. A compound of Embodiment 17a wherein R¹ is C₁-C₈ alkyl optionally substituted with halogen; or a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁴.
Embodiment 18. A compound of Formula 1 or any one of Embodiments 1-17b wherein R¹ is a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected, from C(═O) and C(═S) and the sulfur atom ring members are independently selected from S(═O)ᵤ(═NR²⁴)_{z}, each ring or ring system optionally substituted with up to 5 substituents independently selected from R¹⁴.
Embodiment 18a. A compound of Embodiment 18 wherein R¹ is a phenyl ring, a 5- or 6-membered heteroaromatic ring, or a 5- or 6-membered non-aromatic ring-optionally substituted with up to 3 substituents independently selected from R¹⁴.
Embodiment 18b. A compound of Embodiment 18 wherein R¹ is a phenyl ring or a 5- or 6-membered heteroaromatic ring optionally substituted with up to 3 substituents independently selected from R¹⁴.
Embodiment 18c. A compound of Embodiment 18 wherein R¹ is a 5- or 6-membered non-aromatic ring optionally substituted with up to 3 substituents independently selected from R¹⁴.
Embodiment 18d. A compound of Embodiment 18b wherein R¹ is phenyl, pyridinyl, or thienyl each optionally substituted with up to 3 substituents selected from R¹⁴.
Embodiment 18e. A compound of Embodiment 18b wherein R¹ is phenyl or pyridinyl, each optionally substituted with up to 3 substituents selected from R¹⁴.
Embodiment 18f. A compound of Embodiment 18e wherein R¹ is phenyl optionally-substituted with up to 3 substituents independently selected from R¹⁴.
Embodiment 18g. A compound of Embodiment 18e wherein R¹ is pyridinyl optionally substituted with up to 3 substituents independently selected, from R¹⁴.
Embodiment 18h. A compound of Embodiment 18d wherein R¹ is thienyl optionally substituted with up to 3 substituents independently selected from R¹⁴.
Embodiment 19. A compound of Formula 1 or any one of Embodiments 1-18h wherein R² is C(═O)R¹⁸, CH₂OR¹¹, C(═O)OR¹⁸ or C₁-C₈ alkyl optionally substituted with halogen; or a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected, from R¹⁵.
Embodiment 19a. A compound of Formula 1 or any one of Embodiments 1-18h wherein R² is CH₂OR¹¹ or C(═O)OR¹⁸ optionally substituted with halogen.
Embodiment 19b. A compound of Formula 1 or any one of Embodiments 1-18h wherein R² is C(═O)R¹⁸ or C₁-C₈ alkyl optionally substituted with halogen; or a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected, from C(═O) and C(═S) and the sulfur atom ring members are independently selected, from S(═O)ᵤ(═NR²⁴)_{z}, each ring or ring system optionally substituted, with up to 5 substituents independently selected from R¹⁵.
Embodiment 19c. A compound of Embodiment 19b wherein the radical described as C₁-C₈ alkyl optionally substituted, with halogen is CF₃.
Embodiment 19d. A compound of Embodiment 19 wherein R² is C(═O)R¹⁸, C(═O)OR¹⁸; or a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and O(═S) and the sulfur atom ring members are independently selected from S(═O)ᵤ(═NR²⁴)_{z}, each ring or ring system optionally substituted with up to 5 substituents independently selected, from R¹⁵.
Embodiment 19e. A compound of Embodiment 19d wherein R² is C(═O)OR¹⁸.
Embodiment 19f. A compound of Embodiment 19c wherein R² is C(═O)R¹⁸; or a 3- to 10-membered ring or a 7- to 11-membered ring system, each ring or ring system containing ring members selected from carbon atoms and up to 4 heteroatoms independently selected, from up to 2 O, up to 2 S, and up to 4 N, wherein up to 3-carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁵.
Embodiment 20. A compound of Embodiment 19f wherein R² is a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁵.
Embodiment 20a. A compound of Embodiment 20 wherein R² is a phenyl or 5- or 6-membered heteroaromatic ring optionally substituted with up to 5 substituents independently selected from R¹⁵.
Embodiment 20b. A compound of Embodiment 20a wherein R² is a 5- or 6-membered heteroaromatic ring optionally substituted with up to 5 substituents independently selected from R¹⁵.
Embodiment 20c. A compound of Embodiment 20b wherein R² is pyridinyl, pyrimidinyl or thiazolyl, each optionally substituted with up to 3 substituents independently selected from R¹⁵.
Embodiment 20d. A compound of Embodiment 20c wherein R² is pyridinyl optionally substituted with halogen.
Embodiment 20e. A compound of Embodiment 20d wherein R² is 6-chloro-3-pyridinyl.
Embodiment 20f. A compound of Embodiment 20c wherein R² is thiazolyl optionally substituted with halogen.
Embodiment 20g. A compound of Embodiment 20f wherein R² is 2-chloro-5-thiazolyl.
Embodiment 20h. A compound of Embodiment 20c wherein R² is 6-chloro-3-pyridinyl or 2-chloro-5-thiazolyl.
Embodiment 20i. A compound of Embodiment 20c wherein R² is pyrimidinyl.
Embodiment 20j. A compound of Embodiment 20c wherein R² is 2-methyl-pyrimidin-5-yl.
Embodiment 20k. A compound of Embodiment 20c wherein R² is 6-fluoro-3-pyridinyl.
Embodiment 20l. A compound of Embodiment 20c wherein R² is 6-methyl-3-pyridinyl.
Embodiment 21. A compound of Formula 1 or any one of Embodiments 1-20l wherein each R^{5a} and R^{5b} is independently H, halogen, cyano, hydroxy, amino, nitro, —OCN, —SCN, CHO, C(═O)OH, C(═O)NH₂, C(═S)NH₂, SO₂NH₂, or C₁-C₆ alkyl.
Embodiment 21a. A compound of Formula 1 or any one of Embodiments 1-20l wherein each R^{5a} and R^{5b} is independently H, halogen, cyano, hydroxy, amino, nitro, —OCN, —SCN, CHO, C(═O)OH, C(═O)NH₂, C(═S)NH₂ or SO₂NH₂.
Embodiment 21b. A compound of Formula 1 or any one of Embodiments 1-20l wherein each R^{5a} and R^{5b} is independently C₁-C₆ alkyl.
Embodiment 21c. A compound of Embodiment 21a wherein each R^{5a} and R^{5b} is independently H, halogen or methyl.
Embodiment 21d. A compound of Embodiment 21a wherein each R^{5a} and R^{5b} is independently H or halogen.
Embodiment 21e. A compound of Formula 1 or any one of Embodiments 1-21d wherein each R^{5b} is H.
Embodiment 21f. A compound of Embodiment 21e wherein each R^{5a} and R^{5b} is H.
Embodiment 21g. A compound of Embodiment 21c wherein each R^{5a} and R^{5b} is methyl.
Embodiment 22. A compound of Formula 1 or any one of Embodiments 1-25a wherein each R⁶, R⁷ and R⁸ is independently H, C₁-C₆ alkyl, C₂-C₆ alkylcarbonyl or C₂-C₆ alkoxycarbonyl.
Embodiment 22a. A compound of Embodiment 22 wherein each R⁶, R⁷ and R⁸ is independently H, CH₃, C(═O)CH₃ or C(═O)OCH₃.
Embodiment 22b. A compound of Formula 1 or any one of Embodiments 1-22a wherein each R¹⁰ is independently C₁-C₄ alkyl unsubstituted or substituted with at least one substituent independently selected from the group consisting of halogen and cyano.
Embodiment 22c. A compound of Formula 1 or any one of Embodiments 1-22b wherein each R¹¹ is independently C₁-C₄ alkyl unsubstituted or substituted, with at least one substituent independently selected from the group consisting of halogen and cyano.
Embodiment 23. A compound of Formula 1 or any one of Embodiments 1-22a wherein each R¹⁴ is independently halogen, cyano, SF₅, CHO, C(═O)R¹⁸, C(═O)OR¹⁸, C(═O)NR¹⁸R¹⁹, C(—NOR²¹)R²², Z¹Q^{t} or Z¹QⁱZ¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio or C₂-C₈ alkynylthio, each unsubstituted or substituted with at least one substituent independently selected from R¹⁷.
Embodiment 23a. A compound of Embodiment 23 wherein each R¹⁴ is independently halogen, cyano, SF₅, CHO, C(═O)R¹⁸, C(═O)OR¹⁸, C(═O)NR¹⁸R¹⁹, C(═NOR²¹)R²², Z¹Q^{t} or Z¹QⁱZ¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio or C₂-C₈ alkynylthio, each unsubstituted or substituted with at least one substituent independently selected from R¹⁷.
Embodiment 23b. A compound of Embodiment 23 wherein each R¹⁴ is independently halogen, cyano, SF₅, CHO, C(═O)R¹⁸, C(═O)OR¹⁸, C(═O)NR¹⁸R¹⁹, Z¹Q^{t} or Z¹QⁱZ¹Q¹; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio or C₂-C₈ alkynylthio, each unsubstituted or substituted with at least one substituent independently selected from R¹⁷.
Embodiment 23c. A compound of Embodiment 23a wherein each R¹⁴ is independently halogen, cyano, C(═O)OR¹⁸, C(═O)NR¹⁸R¹⁹, C(═NOR²¹)R²², Z¹Q^{t}; or C₁-C₈ alkyl, C₁-C₈ alkoxy, C₁-C₈ alkylthio, or C₁-C₈ alkylsulfinyl each optionally substituted with halogen.
Embodiment 23d. A compound of Embodiment 23b wherein each R¹⁴ is independently halogen, eyano, C(═O)OR¹⁸, C(═O)NR¹⁸R¹⁹, C(═NOR²¹)R²², Z¹Q^{t}; or C₁-C₈ alkyl, C₁-C₈ alkoxy or C₁-C₈ alkylthio, each optionally substituted with halogen.
Embodiment 23e. A compound of Embodiment 23c wherein each R¹⁴ is independently halogen, cyano, C(═NOR²¹)R²², Z¹Q^{t}; or C₁-C₈ alkyl, C₁-C₈ alkoxy or C₁-C₈ alkylthio, each optionally substituted with halogen.
Embodiment 23f. A compound of Embodiment 23c wherein each R¹⁴ is independently halogen, Z¹Q^{t}; or C₁-C₈ alkyl, C₁-C₈ alkoxy or C₁-C₈ alkylthio, each optionally substituted with halogen.
Embodiment 23g. A compound of Formula 1 or any one of Embodiments 1-23d wherein the ring or ring system of R¹ is substituted with 1 to 5 substituents independently selected from R¹⁴ wherein one of said substituents is C(═NOR²¹)R²².
Embodiment 23h. A compound of Formula 1 or any one of Embodiments 1-23d wherein the ring or ring system of R¹ is substituted with 1 to 5 substituents independently selected, from R¹⁴ wherein one of said substituents is cyano.
Embodiment 23i. A compound of Formula 1 or any one of Embodiments 1-23d wherein the ring or ring system of R¹ is substituted with 1 to 5 substituents independently selected from R¹⁴ wherein one of said substituents is C₁-C₈ alkylsulfinyl.
Embodiment 24. A compound of Formula 1 or any one of Embodiments 1-23c wherein each R¹⁵ is independently halogen, cyano, SF₅, CHO, C(═O)R¹⁸, C(═O)OR¹⁸, C(═O)NR¹⁸R¹⁹, Z¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cyeloalkylthio, C₄-C₁₀ cycloalkylalkylthio, C₂-C₈ alkenylthio and C₃-C₈ alkynylthio, each unsubstituted or substituted with at least one substituent independently selected from R¹⁷.
Embodiment 24a. A compound of Embodiment 24 wherein each R¹⁵ is independently halogen or C₁-C₄ alkyl.
Embodiment 25. A compound of Formula 1 or any one of Embodiments 1-24a wherein each R¹⁶ is independently C₁-C₄ alkyl unsubstituted or substituted with at least one substituent independently selected from the group consisting of halogen and cyano.
Embodiment 25a. A compound of Formula 1 or any one of Embodiments 1-24a wherein each R¹⁷ is independently halogen, OR¹¹ or Z¹Q^{t}.
Embodiment 26. A compound of Formula 1 or any one of Embodiments 1-25 wherein each Z¹ is a direct bond.
Embodiment 27. A compound of Formula 1 or any one of Embodiments 1-26 wherein a is 1.
Embodiment 28. A compound of Formula 1 or any one of Embodiments 1-27 wherein each R²⁴ is independently H, cyano, C₁-C₄ alkyl or C₁-C₄ alkoxy.
Embodiment 29. A compound of Formula 1 or any one of Embodiments 1-28 wherein R^{29a} is H.
Embodiment 30. A compound of Formula 1 or any one of Embodiments 1-29 wherein R^{29b} is H or F.
Embodiment 31. A compound of Formula 1 or any one of Embodiments 1-30 wherein in each instance of S(═O)ᵤ(═NR²⁴)_{z}, when z is 1 then u is 1.
Embodiment 32. A compound of Formula 1 or any one of Embodiments 1-31 wherein each Qⁱ and Q^{t} is independently a 5- to 6-membered ring, each ring containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring optionally substituted with up to 5 substituents independently selected from the group consisting of halogen, cyano, OR¹¹, S(O)ₙR¹⁰ and R¹⁶.
Embodiment 32a. A compound of Embodiments 32 wherein each Qⁱ and Q^{t} is independently phenyl, pyridinyl, pyrimidinyl, thienyl, thiazolyl and isoxazolinyl each ring optionally substituted with up to 5 substituents independently selected from the group consisting of halogen, cyano, OR¹¹, S(O)ₙR¹⁰ and R¹⁶.
Embodiment 32b. A compound of Embodiments 32a wherein each Qⁱ and Q^{t} is independently phenyl, pyridinyl, pyrimidinyl, thienyl, thiazolyl and isoxazolinyl each ring optionally substituted with up to 5 substituents independently selected from the group consisting of halogen, cyano, OR¹¹, S(O)ₙR¹⁰ and R¹⁶.
Embodiment 32c. A compound of Embodiments 32b wherein each Qⁱ and Q^{t} is independently phenyl optionally substituted with up to 5 substituents independently selected from the group consisting of halogen, cyano, OR¹¹, S(O)ₙR¹⁰ and R¹⁶.
Embodiment 32d. A compound of Embodiments 32b wherein each Qⁱ and Q^{t} is independently pyridinyl optionally substituted with up to 5 substituents independently selected from the group consisting of halogen, cyano, OR¹¹, S(O)ₙR¹⁰ and R¹⁶.
Embodiment 32e. A compound of Embodiments 32b wherein each Qⁱ and Q^{t} is independently pyrimidinyl optionally substituted with up to 5 substituents independently selected from the group consisting of halogen, cyano, OR¹¹, S(O)ₙR¹⁰ and R¹⁶.
Embodiment 32f. A compound of Embodiments 32b wherein each Qⁱ and Q^{t} is independently thienyl optionally substituted with up to 5 substituents independently selected from the group consisting of halogen, cyano, OR¹¹, S(O)ₙR¹⁰ and R¹⁶.
Embodiment 32g. A compound of Embodiments 32b wherein each Qⁱ and Q^{t} is independently thiazolyl optionally substituted, with up to 5 substituents independently selected from the group consisting of halogen, cyano, OR¹¹, S(O)ₙR¹⁰ and R¹⁶.
Embodiment 32h. A compound of Embodiments 32b wherein each Qⁱ and Q^{t} is independently isoxazolinyl optionally substituted with up to 5 substituents independently selected from the group consisting of halogen, cyano, OR¹¹, S(O)ₙR¹⁰ and R¹⁶.
Embodiment 32i. A compound of Formula 1 or any one of Embodiments 1-31 wherein each Qⁱ and Q^{t} is independently phenyl or pyridinyl, each optionally substituted with up to 5 substituents independently selected from the group consisting of halogen and C₁-C₄ alkyl.

Of note is a compound of Formula 1 or any one of Embodiments 1-32i wherein X and Y are O, a composition comprising said compound, and its use for controlling an invertebrate pest.

Embodiments of this invention, including Embodiments 1-32i above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-32i above as well as any other embodiments described herein, and any combination thereof, pertain to the compositions and methods of the present invention.

Combinations of Embodiments 1-32i are illustrated by:

Embodiment A. A compound of Formula 1 wherein
A is C(R^{29a})═C(R^{29b}), S or NCH₃.
X is O;
Y is O;
Z is a direct bond, O or NR⁶;
R¹ is C₁-C₈ alkyl optionally substituted with halogen; or a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁴;
R² is C(═O)OR¹⁸; or C₁-C₈ alkyl optionally substituted with halogen; or a 3- to 10-membered ring or a 7- to 11-membered ring system, each ring or ring system containing ring members selected from carbon atoms and up to 4 heteroatoms independently selected, from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁵;
R³ is H, C₁-C₆ alkyl, C₁-C₆ haloalkyl, C₂-C₆ alkenyl, C₂-C₆ haloalkenyl, C₂-C₆ alkynyl, C₂-C₆ haloalkynyl or C≡CR²⁵; or C₃-C₆ cycloalkyl or C₄-C₇ cycloalkylalkyl, each optionally substituted, with up to 4 substituents independently selected from the group consisting of halogen, C₁-C₂ alkyl, 1 cyclopropyl and 1 CF₃;
R₄ is C₁-C₆ alkyl, C₁-C₆ haloalkyl, C₂-C₆ alkenyl, C₂-C₆ haloalkenyl, C₂-C₆ alkynyl, C₂-C₆ haloalkynyl or C≡CR²⁵; or C₃-C₆ cycloalkyl or C₄-C₇ cycloalkylalkyl, each optionally substituted with up to 4 substituents independently selected from the group consisting of halogen, C₁-C₂ alkyl, 1 cyclopropyl and 1 CF₃; or
R³ and R⁴ are taken together with the contiguous linking nitrogen and carbon atoms to form an optionally substituted, ring R-1 or ring R-2;
R^{5a} and R^{5b} are H; and

a is 1.

Embodiment B. A compound of Embodiment A wherein
A is C(R^{29a})═C(R^{29b}) or NCH₃;
Z is a direct bond;
R¹ is phenyl optionally substituted with up to 3 substituents independently selected, from R¹⁴;
R² is pyridinyl, pyrimidinyl or thiazolyl, each optionally substituted with up to 3 substituents independently selected from R¹⁵;
R³ is C₁-C₆ alkyl, C₁-C₆ haloalkyl or cyclopropyl;
R⁴ is C₁-C₆ alkyl, C₁-C₆ haloalkyl or cyclopropyl; or
R³ and R⁴ are taken together with the contiguous linking nitrogen and carbon atoms to form the optionally substituted ring R-1 or ring R-2;
each R¹⁴ is independently halogen, cyano, SF₅, CHO, C(═O)R¹⁸, C(═O)OR¹⁸, C(═O)NR¹⁸R¹⁹, Z¹Q^{t} or Z¹QⁱQ^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio or C₂-C₈ alkynylthio, each unsubstituted or substituted, with at least one substituent independently selected from R¹⁷;
each R¹⁵ is independently halogen, cyano, SF⁵, CHO, C(═O)R¹⁸, C(═O)OR¹⁸, C(═O)NR¹⁸R¹⁹, Z¹Q^{t}; or C₁-C₈ alkyl, C₂-C₈ alkenyl, C₂-C₈ alkynyl C₃-C₁₀ cycloalkyl C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio and C₂-C₈ alkynylthio, each unsubstituted or substituted with at least one substituent independently selected from R¹⁷;
R¹⁷ is halogen, OR¹¹ or Z¹Q^{t};
R²⁸ is CH₃; and
Z¹ is a direct bond;
each Qⁱ and Q^{t} is independently phenyl or pyridinyl each optionally substituted with up to 5 substituents independently selected from the group consisting of halogen and C₁-C₄ haloalkyl;
m is 2; and
p is 1.

Embodiment C. A compound of Embodiment A wherein
A is C(R^{29a})═C(R^{29b}) or NCH₃;
Z is a direct bond;
R¹ is a phenyl ring, a 5- or 6-membered heteroaromatic ring, or a 5- or 6-membered non-aromatic ring optionally substituted with up to 3 substituents independently selected from R¹⁴;
R² is pyridinyl, pyrimidinyl or thiazolyl, each optionally substituted with up to 3 substituents independently selected from R¹⁵;
R³ is C₁-C₆ alkyl, C₁-C₆ haloalkyl or cyclopropyl;
R⁴ is C₁-C₆ alkyl, C₁-C₆ haloalkyl or cyclopropyl; or
R³ and R⁴ are taken together with the contiguous linking nitrogen and carbon atoms to form the optionally substituted ring R-1 or ring R-2;
each R¹⁴ is independently halogen, cyano, SF₅, CHO, C(═O)R¹⁸, C(═O)OR¹⁸, C(═O)NR¹⁸R¹⁹, C(═NOR²¹)R²², Z¹Q^{t} or Z¹QⁱZ¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio or C₂-C₈ alkynylthio, each unsubstituted or substituted with at least one substituent independently selected from R¹⁷;
each R¹⁵ is independently halogen, cyano, SF₅, CHO, C(═O)R¹⁸, C(═O)OR¹⁸, C(═O)NR¹⁸R¹⁹, Z¹Q^{t}; or C₁-C₈ alkyl, C₂-C₈ alkenyl, C₂-C₈ alkynyl, C₃-C₁₀ -cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio and C₂-C₈ alkynylthio, each unsubstituted or substituted with at least one substituent independently selected from R¹⁷;
R¹⁷ is halogen, OR¹¹ or Z¹Q^{t};
R²⁸ is CH₃; and
Z¹ is a direct bond;
each Qⁱ and Qₜ is independently a 5- to 6-membered ring, each ring containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring optionally substituted with up to 5 substituents independently selected from the group consisting of halogen, cyano, OR¹¹, S(O)ₙR¹⁰ and R¹⁶;
m is 2; and
p is 1.

Embodiment D. A compound of Embodiment C wherein
R¹ is phenyl optionally substituted with up to 3 substituents independently selected from R¹⁴;
R³ and R⁴ are taken together with the contiguous linking nitrogen and carbon atoms to form the optionally substituted ring R-2; and
each Qⁱ and Q^{t} is independently phenyl or pyridinyl, each optionally substituted with up to 5 substituents independently selected from the group consisting of halogen and C₁-C₄ haloalkyl.

Embodiment E. A compound of Embodiment A wherein
A is C(R^{29a})═(R^{29b}) or NCH₃;
Z is a direct bond;
R¹ is a phenyl, pyridinyl, or thienyl each optionally substituted with up to 3 substituents selected from R¹⁴;
R² is pyridinyl, pyrimidinyl or thiazolyl, each optionally substituted with up to 3 substituents independently selected from R¹⁵;
R³ is C₁-C₆ alkyl, C₁-C₆ haloalkyl or cyclopropyl;
R⁴ is C₁-C₆ alkyl, C₁-C₆ haloalkyl or cyclopropyl; or
R³ and R⁴ are taken together with the contiguous linking nitrogen and carbon atoms to form the optionally substituted ring R-1 or ring R-2;
each R¹⁴ is independently halogen, cyano, SF₅, CHO, C(═O)R¹⁸, C(═O)OR¹⁸, C(═O)NR¹⁸R¹⁹, C(═NOR²¹)R²², Z¹Q^{t} or Z¹QⁱZ¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio or C₂-C₈ alkynylthio, each unsubstituted or substituted with at least one substituent independently selected from R¹⁷;
each R¹⁵ is independently halogen, cyano, SF₅, CHO, C(═O)R¹⁸, C(═O)OR¹⁸, C(═O)NR¹⁸R¹⁹, Z¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio and C₂-C₈ alkynylthio, each unsubstituted or substituted with at least one substituent independently selected from R¹⁷;
R¹⁷ is halogen, OR¹¹ or Z¹Q¹;
R²⁸ is CH₃;
Z¹ is a direct bond;
each Q⁸ and Q^{t} is independently a 5- to 6-membered ring, each ring containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring optionally substituted with up to 5 substituents independently selected from the group consisting of halogen, cyano, OR¹¹, S(O)ₙR¹⁰ and R¹⁶;
each R¹⁶ is independently C₁-C₄ alkyl unsubstituted or substituted with, at least one substituent independently selected from the group consisting of halogen and cyano;
each R¹⁰ is independently C₁-C₄ alkyl unsubstituted or substituted with at least one substituent independently selected from the group consisting of halogen and cyano;
each R¹¹ is independently C₁-C₄ alkyl unsubstituted or substituted with, at least one substituent independently selected from the group consisting of halogen and cyano;
m is 2; and
p is 1.

Embodiment F. A compound, of Embodiment A wherein
A is C(R^{29a})═C(R^{29b}) or NCH₃;
Z is a direct bond;
R¹ is phenyl optionally substituted with up to 3 substituents independently selected from R¹⁴;
R² is pyridinyl, pyrimidinyl or thiazolyl, each optionally substituted with up to 3 substituents independently selected from R¹⁵;
R³ and R⁴ are taken together with the contiguous linking nitrogen and carbon atoms to form the optionally substituted ring R-2;
each R¹⁴ is independently halogen, cyano, SF₅, CHO, C(═O)R¹⁸, C(═O)OR¹⁸, C(═O)NR¹⁸R¹⁹, C(═NOR²¹)R²², Z¹Q^{t} or Z¹QⁱZ¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio or C₂-C₈ alkynylthio, each unsubstituted or substituted with at least one substituent independently selected, from R¹⁷;
each R¹⁵ is independently halogen, cyano, SF₅, CHO, C(═O)R¹⁸, C(═O)OR¹⁸, C(═O)NR¹⁸R¹⁹, Z¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio and C₂-C₈ alkynylthio, each unsubstituted or substituted with at least one substituent independently selected from R¹⁷;
R¹⁷ is halogen, OR¹¹ or Z¹Q^{t};
R²⁸ is CH₃;
Z¹ is a direct bond;
each Qⁱ and Q^{t} is independently phenyl or pyridinyl, each optionally substituted with up to 5 substituents independently selected from the group consisting of halogen and C₁-C₄ haloalkyl;
m is 2; and
p is 1.

Embodiment G. A compound, of Embodiment B wherein
R³ is CH₃, CH₂CH₃ or cyclopropyl;
R⁴ is CH₃; or
R³ and R⁴ are taken together with the contiguous linking nitrogen and carbon atoms to form the optionally substituted ring R-2;
each R¹⁴ is independently halogen, Z¹Q^{t}; or C₁-C₈ alkyl, C₁-C₈ alkoxy or C₁-C₈ alkylthio, each optionally substituted with halogen;
each R¹⁵ is independently halogen or C₁-C₄ alkyl;
R^{29a} is H; and
R^{29b} is H or F.

Embodiment H. A compound of Embodiment B wherein
R³ is CH₃, CH₂CH₃ or cyclopropyl;
R⁴ is CH₃; or
R³ and R⁴ are taken together with the contiguous linking nitrogen and carbon atoms to form the optionally substituted ring R-2;
each R¹⁴ is independently halogen, cyano, C(═NOR²¹)R²², Z¹Q^{t}; or C₁-C₈ alkyl, C₁-C₈ alkoxy or C₁-C₈ alkylthio, each optionally substituted with halogen;
each R¹⁵ is independently halogen or C₁-C₄ alkyl;
R^{29a} is H; and
R^{29b} is H or F.

Embodiment I. A compound of Embodiment F wherein
A is C(R^{29a})═C(R^{29b});
each R¹⁴ is independently halogen, cyano, C(═O)OR¹⁸, C(═O)NR¹⁸R¹⁹, C(═NOR²¹)R²², Z¹Q^{t}; or C₁-C₈ alkyl, C₁-C₈ alkoxy or C₁-C₈ alkylthio, each optionally substituted with halogen;
each R¹⁵ is independently halogen or C₁-C₄ alkyl;
R^{29a} is H; and
R^{29b} is H or F.

Specific embodiments include compounds of Formula 1 selected from the group consisting of:
1-[(5-chloro-2-thienyl)methyl]-2-hydroxy-4-oxo-3-phenyl-4H-pyrido[1,2-a]pyrimidinium inner salt;
2-hydroxy-4-oxo-3-phenoxy-1-propyl-4H-pyrido[1,2-a]pyrimidinium inner salt;
1-[(4-chlorophenyl)methyl]-2-hydroxy-4-oxo-3-[3-(trifluoromethoxy)phenyl]4H-pyrido[1,2-a]pyrimidinium inner salt;
1-[(6-chloro-3-pyridixiyl)methyl]-2-hydroxy-4-oxo-3-[3-[[(2,2,2-trifluoroethyl)amino]carbonyl]phenyl]-4H-pyrido[1,2-a]pyrimidinium inner salt;
2-hydroxy-4-oxo-1-[2-(3-pyridinyl)ethyl]-3-[3-(trifluoroethoxy)phenyl]-4H-pyrido[1,2-a]pyrimidinium inner salt;
3-(4-fluorophenyl)-2-hydroxy-4-oxo-1-[2-(2-pyridinyl)ethyl]-4H-pyrido[1,2-a]pyrimidinium inner salt;
1-[(6-chloro-3-pyridinyl)methyl]-3-(2,3-dihydro-1H-inden-1-yl)-2-hydroxy-4-oxo-4H-pyrido[1,2-a]pyrimidinium inner salt;
1-[(2-chloro-5-thiazolyl)methyl]-3-(2,3-dihydro-1H-inden-1-yl)-2-hydroxy-4-oxo-4H-pyrido[1,2-a]pyrimidinium inner salt;
2-hydroxy-1-methyl-4-oxo-3-phenyl-4H-pyrido[1,2-a]pyrimidinium inner salt;
1-[2-(acetylamino)ethyl]-2-hydroxy-4-oxo-3-phenyl-4H-pyrido[1,2-a]pyrimidimium inner salt;
3-[3-(cyanomethoxy)phenyl]2-hydroxy-4-oxo-1-(2,2,2-trifluoroethyl)-4H-pyrido[1,2-a]pyrimidinium inner salt;
1-[(6-chloro-3-pyridinyl)methyl]-3-[3-(cyanomethoxy)phenyl]-2-hydroxy-4-oxo-4H-pyrido[1,2-a]pyrimidinium inner salt;
3-[2-[(6-chloro-3-pyridmyl)methoxy]-4-fluorophenyl]-2-hydroxy-4-oxo-1-(2,2,2-trifluoroethyl)-4H-pyrido[1,2-a]pyrimidinium inner salt;
3-[3-[(6-chloro-3-pyridinyl)methoxy]phenyl]-2-hydroxy-4-oxo-1-(2,2,2-trifluoroethyl)-4H-pyrido[1,2-a]pyrimidinium inner salt;
3-[3-[(6-chloro-3-pyridinyl)methoxy]phenyl]-1-[(6-chloro-3-pyridinyl)methyl]-2-hydroxy-4-oxo-4H-pyrido[1,2-a]pyrimidinium inner salt;
1-[(6-chloro-3-pyridinyl)methyl]-2-hydroxy-4-oxo-3-[3-(2,2,2-trifluoroethoxy)phenyl]-4H-pyrido[1,2-a]pyrimidinium inner salt;
1-[(6-chloro-3-pyridmyl)methyl]-3-[4-fluoro-3-(2-pyridinylmethoxy)phenyl]-2-hydroxy-4-oxo-4H-pyrido[1,2-a]pyrimidinium inner salt;
3-[3-[(6-chloro-2-pyridinyl)methoxy]phenyl]-2-hydroxy-4-oxo-1-(2,2,2-trifluoroethyl)-4H-pyrido[1,2-a]pyrimidinium inner salt;
2-hydroxy-1-[(4-methoxyphenyl)methyl]-4-oxo-3-phenyl-4H-pyrido[1,2-a]pyrimidinium inner salt;
3-[3-(3-bromo-4,5-dihydro-5-isoxazolyl)phenyl]-1-[(2-chloro-5-thiazolyl)methyl]-2-hydroxy-4-oxo-4H-pyrido[1,2-a]pyrimidinium inner salt;
1-[(2-chloro-5-thiazolyl)methyl]-3-[3-(4,5-dihydro-3-methyl-5-isoxazoiyl)phenyl]-2-hydroxy-4-oxo-4H-pyrido[1,2-a]pyrimidinium inner salt;
1-(1,3-dioxolan-2-ylmethyl)-2-hydroxy-4-oxo-3-phenyl-4H-pyrido[1,2-a]pyrimidinium inner salt;
1-(1,4-dioxan-2-ylmethyl)-2-hydroxy-4-oxo-3-phenyl-4H-pyrido[3,2-a]pyrimidinium inner salt; and
1-[(2-chloro-5-thiazolyl)methyl]-2-hydroxy-3-[3-[1-(methoxyimino)ethyl]phenyl]-4-oxo-4H-pyrido[1,2-a]pyrimidinium inner salt.

Further specific embodiments include compounds of Formula 1 selected from the group consisting of compound numbers 102, 105, 107 and 113, wherein the compound number refers to compounds in Index Table A.

Of note is that compounds of this invention are characterized by favorable metabolic and/or soil residual patterns and exhibit activity controlling a spectrum of agronomic and nonagronomic invertebrate pests.

Of particular note, for reasons of invertebrate pest control spectrum and economic importance, protection of agronomic crops from damage or injury caused by invertebrate pests by controlling invertebrate pests are embodiments of the invention. Compounds of this invention because of their favorable translocation properties or systemicity in plants also protect foliar or other plant parts which are not directly contacted with a compound of Formula 1 or a composition comprising the compound.

Also noteworthy as embodiments of the present invention are compositions comprising a compound of any of the preceding Embodiments, as well as any other embodiments described herein, and any combinations thereof, and at least one additional component selected, from the group consisting of a surfactant, a solid diluent and a liquid diluent, said compositions optionally further comprising at least one additional biologically active compound or agent.

Further noteworthy as embodiments of the present invention are compositions for controlling an invertebrate pest comprising a biologically effective amount of a compound of any of the preceding Embodiments, as well as any other embodiments described herein, and any combinations thereof, and at least one additional component selected from the group consisting of a surfactant, a solid diluent and a liquid diluent, said compositions optionally further comprising a biologically effective amount of at least one additional biologically active compound or agent.

Embodiments of the invention also include a composition for protecting an animal comprising a compound, (i.e. in a parasiticidally effective amount) of any of the preceding Embodiments and a carrier.

Embodiments of the invention further include methods for controlling an invertebrate pest comprising contacting the invertebrate pest or its environment with a biologically effective amount of a compound, of any of the preceding Embodiments (e.g., as a composition described herein). Of particular note is a method for protecting an animal comprising administering to the animal a parasiticidally effective amount of a compound of any of the preceding Embodiments (e.g., as a composition described herein).

Embodiments of the invention also include a composition comprising a compound of any of the preceding Embodiments in the form of a soil drench liquid formulation. Embodiments of the invention further include methods for controlling an invertebrate pest comprising contacting the soil with a liquid composition as a soil drench comprising a biologically effective amount of a compound of any of the preceding Embodiments.

Embodiments of the invention also include a spray composition for controlling an invertebrate pest comprising a biologically effective amount of a compound of any of the preceding Embodiments and a propellant. Embodiments of the invention further include a bait composition for controlling an invertebrate pest comprising a biologically effective amount of a compound of any of the preceding Embodiments, one or more food materials, optionally an attractant, and optionally a humectant. Embodiments of the invention also include a device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

Embodiments of the invention also include a method, for protecting a seed from an invertebrate pest comprising contacting the seed with a biologically effective amount of a compound of any of the preceding Embodiments (e.g., as a composition described herein).

Embodiments of the invention also include methods for protecting an animal from an invertebrate parasitic pest comprising administering to the animal a parasiticidally effective amount of a compound of any of the preceding Embodiments.

Embodiments of the invention also include methods for controlling an invertebrate pest comprising contacting the invertebrate pest or its environment with a biologically effective amount of a compound of Formula 1, an N-oxide or a salt thereof, (e.g., as a composition described herein), provided that the methods are not methods of medical treatment of a human or animal body by therapy.

This invention also relates to such methods wherein the invertebrate pest or its environment is contacted with a composition comprising a biologically effective amount of a compound of Formula 1, an N-oxide or a salt thereof, and at least one additional component selected from the group consisting of surfactants, solid diluents and liquid diluents, said composition optionally further comprising a biologically effective amount of at least one additional biologically active compound or agent, provided that the methods are not methods of medical treatment of a human or animal body by therapy.

One or more of the following methods and variations as described in Schemes 1-13 can be used to prepare the compounds of Formula 1. The definitions of R¹, R², R³, R⁴, R^{5a}, R^{5b} and Z in the compounds of Formulae 1-13 below are as defined, above in the Summary of the Invention unless otherwise noted. Formulae 1a-1e are various subsets or analogs of Formula 1, and all substituents for Formulae 1a-1e are as defined above for Formula 1 unless otherwise indicated. Ambient or room temperature is defined as about 20-25° C.

Compounds of Formula 1a (i.e. Formula 1 wherein X and Y are O) can be prepared by condensation of appropriately substituted compounds of Formula 2 with optionally substituted malonic acids (3a) in the presence of condensing agents as shown in Scheme 1. Condensing agents can be carbodiimides such as dicyclohexyl carbodiimide (see, for example, Koch, A. et al. Tetrahedron 2004, 60, 10011-10018) or other agents well known in the art to form amide bonds with or without activating agents such as N-hydroxybenzotriazole as described in Science of Synthesis 2005, 21, 17-25 and Tetrahedron 2005, 61, 10827-10852. This reaction is typically carried out in an inert organic solvent, such as dichloromethane or 1,2-dichloroethane, at temperatures from about 0 to about 80° C. for a period of 10 minutes to several days.

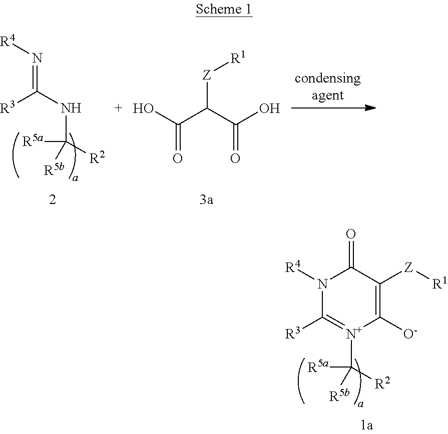

Compounds of Formula 1a can also be prepared by the condensation of compounds of Formula 2 with malonic acid esters (3b) wherein R is a C₁-C₅ alkyl group as shown in Scheme 2. These reactions can be performed neat or in the presence of inert solvents as described in Bulletin of the Chemical Society of Japan 1999, 72(3), 503-509. Inert solvents include, but are not limited to, high boiling hydrocarbons such as mesitylene, tetralin or cymene, or high boiling ethers such as diphenyl ether. Typical temperatures range from 50 to 250° C. Of note are temperatures from 150 to 200° C., which typically provide rapid reaction times and high yields. These reactions can also be performed in microwave reactors within the same temperature ranges. Typical reaction times range from 5 minutes to several hours.

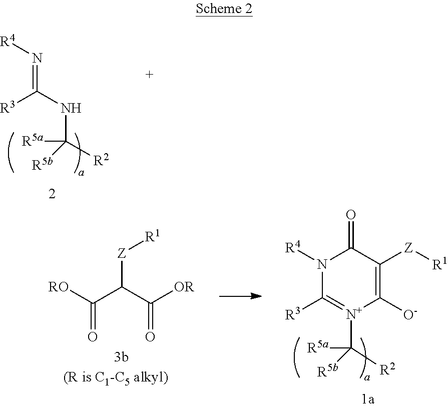

Compounds of Formula 3a can be prepared, by a variety of methods known in the art, for example by base hydrolysis of compounds of Formula 3b.

Compounds of Formula 3b wherein Z is a direct bond and R¹ is an optionally substituted aromatic (including heteroaromatic) ring or ring system can be prepared by arylation of malonate esters (using compounds of formula R¹X¹ wherein X¹ is Cl, Br or I, examples of which are found in Tables I-25 and I-31) catalyzed by palladium (J. Org. Chem. 2002, 67, 541-555) or copper (Org. Lett. 2002, 4, 269-272 and Org. Lett. 2005, 7, 4693-4695). Alternatively, compounds of Formula 3b can be prepared by the method shown in Scheme 2a (see, for example, J. Med. Chem. 1982, 25(6), 745-747).

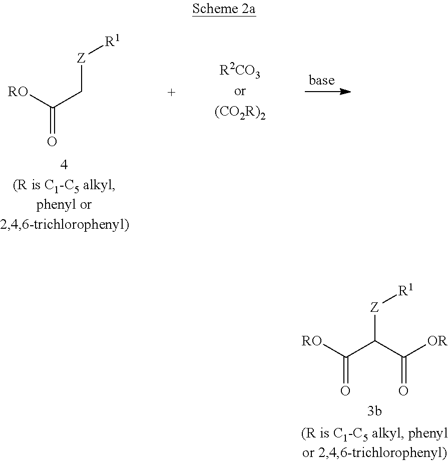

Esters of Formula 4 can be prepared from the corresponding acids by methods well known in the art. Many of the acids of Formula 4 where R is H are commercially available or readily prepared by methods known in the art (examples are listed in Table I-1).

Compounds of Formula 3b can also be prepared by the method shown in Scheme 2b. Reaction of nitrites of Formula 3g with dialkyl carbonates yields nitrite esters of Formula 3h, and subsequent acidic hydrolysis in the presence of an alcohol provides the compounds of Formula 3b (see, for example, Helvetica Chimica Acta 1991, 74(2), 309-314). Many of the nitrites of Formula 3g are commercially available or readily prepared by methods known in the art.

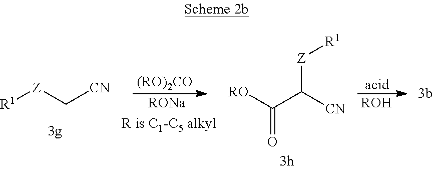

Compounds of Formula 1a can also be prepared by treatment of compounds of Formula 2 with activated esters of Formula 3c wherein LvO is an activated leaving group as shown in Scheme 3. Examples of Lv preferred for ease of synthesis or reactivity are phenyl, 4-nitrophenyl or halogen-substituted, phenyl (e.g., 2,4,6-trichlorophenyl, pentachlorophenyl or pentafluorophenyl) as described in Archiv der Pharmazie (Weinlieim, Germany) 1991, 324, 863-866. Other activated esters are well known in the art and include, but are not limited to, N-hydroxysuccinimide esters (see, for example, J. Am. Chem. Soc. 2002, 124, 6872-6878). Typical temperatures range from 50 to 200° C. Of note are temperatures from 50 to 150° C., which typically provide rapid reaction times and high yields. These reactions can be performed, with or without solvent, such as toluene, and in microwave reactors within the same temperature ranges. Typical reaction times range from 5 minutes to 2 hours.

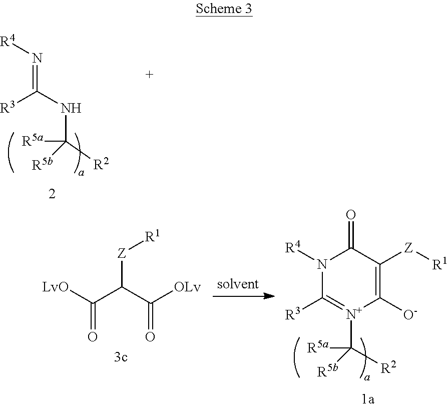

Compounds of the Formula 3c can be prepared, for example, from compounds of Formula 3a (see, for example, J. Met. Chem. 1980, 17, 337).

Compounds of Formula 1a can also be prepared by condensation of compounds of Formula 2 with compounds of Formula 3d or 3e, or by condensation of compounds of Formula 2 with mixtures of compounds of Formulae 3d and 3e as shown in Scheme 4. These reactions are typically performed in an inert solvent, such as dichloromethane, and optionally in the presence of two or more equivalents of an acid, acceptor (see, for example, Zeitschrift für Naturforschung, Tell B: Anorganische Chemie, Organische Chemie 1982, 37B(Z), 222-233). Typical acid acceptors include, but are not limited to, triethylamine, N,N-diisopropylethylamine, pyridine and substituted pyridines.

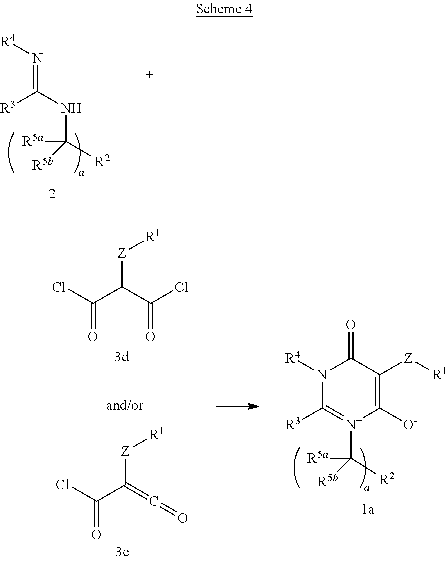

A particularly useful method for the preparation of compounds of Formula 2 is shown in Scheme 5. In the method of Scheme 5, compounds of Formula 2a are protected with suitable protecting groups such as, but not limited to, tert-butoxycarbonyl, acetyl or formyl to form the intermediate of Formula 2b wherein PG is a protecting group. The compound of Formula 2b is then alkylated with an appropriate reagent of Formula 5 (wherein at least one of R^{5a} or R^{5b} is hydrogen and X is a leaving group such as a halogen) to give an intermediate of Formula 2c. The protecting group is removed to provide a compound of Formula 2. Conditions for the formation and removal of protecting groups on an amine function are known in the literature (see, for example, Greene, T. W.; Wuts, P. G. M, Protective Groups in Organic Synthesis, 2nd ed.; Wiley: New York, 1991).

Examples of particularly useful compounds of Formula 2 are shown in Tables I-33 to I-38. Some examples of compounds of Formula 2a are shown in Table I-39.

Compounds of Formula 2 can also be prepared in a variety of ways known in the art; see, for example, Patai, S. The Chemistry of Functional Groups: The Chemistry of Amidines and Imidates; Wiley: Chichester, UK, 1975; The Chemistry of Amidines and Imidates; Patai, S.; Rappoport, Z., Eds.: Wiley: Chichester, UK, 1991; Vol. 2; Mega, T. et al. Bulletin of the Chemical Society of Japan 1988, 67(12), 4315-4321; Ife, R. et al. European Journal of Medicinal Chemistry 1989, 24(3), 249-257; Wagaw, S.; Buchwald, S. Journal of Organic Chemistry 1996, 61(21), 7240-7241; Shen, Q. et al. Angewandie Chemie, International Edition 2005, 44(9), 1371-1375; and Okano, K. et al. Organic Letters 2003, 5(26), 4987-4990.

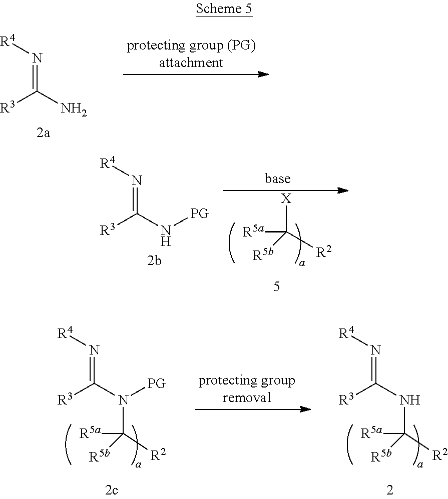

Compounds of Formula 1b (i.e. Formula 1a wherein Z is a direct bond) wherein R¹ is an optionally substituted, aromatic ring or ring system can be prepared, from compounds of Formula 1c (i.e. analogous to Formula 1a wherein Z is a direct bond and R¹ is H) and compounds of Formula 6 wherein X¹ is Cl, Br or I (preferably Br or I) as shown in Scheme 6.

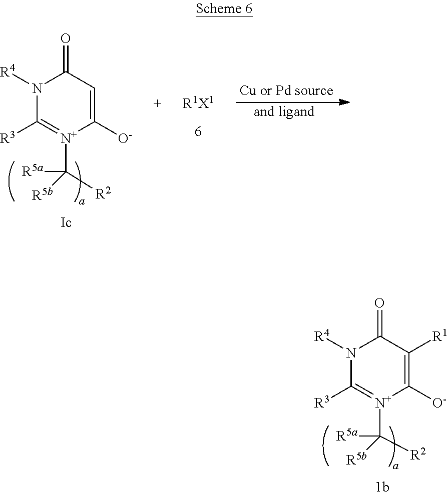

These reactions are typically carried out in the presence of a copper or palladium catalyst preferably under an inert atmosphere. The copper catalysts used for the present method typically comprise copper in metallic form (e.g., as a powder) or copper in a formal oxidation state of 1 (i.e. Cu(I)). Examples of copper-containing compounds useful as catalysts in the method of Scheme 6 include Cu, CuI, CuBr and CuCl. Examples of palladium-containing compounds useful as catalysts in the method of Scheme 6 include Pd(OAc)₂. Useful solvents for the method of Scheme 6 include, for example, ethers such as 1,4-dioxane, amides such as N,N-dimethylacetamide and dimethyl sulfoxide.

The method of Scheme 6 can be conducted over a wide range of temperatures from 25 to 200° C. Of note are temperatures from 40 to 150° C. The method of Scheme 6 can be conducted in the presence of a ligand A wide variety of copper-binding compounds are useful as ligands for the present method. Examples of useful ligands include, but are not limited to, 1,10-phenanthroline, N,N-dimethylethylenediamine, L-proline and 2-picolinic acid. The general methods and procedures for copper-catalyzed Ullmann-type coupling reactions are well known in the literature; see, for example, Xie, Ma, et al. Org. Lett. 2005, 7, 4693-4695.

Compounds of Formula 1d (i.e. Formula 1a wherein Z is S(O)ₙ) can be prepared from compounds of Formula 1c by treatment with compounds of Formula 7, optionally in the presence of a Lewis acid catalyst (e.g., FeCl₃), as shown in Scheme 7. Examples of compounds of Formula 7 useful in the method of Scheme 7 include, but are not limited to, sulfenyl and sulfonyl halides. Typically the reaction is performed in an inert solvent, more typically a polar solvent such as N,N-dimethylacetamide or 1-methyl-2-pyrrolidinone. The reaction is typically performed at temperatures from 0 to 180° C., more typically at ambient temperature to 150° C. Microwave irradiation can be advantageous in heating the reaction mixture.

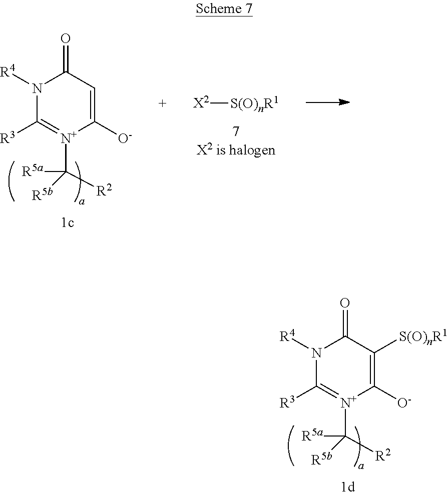

Compounds of Formula 1c (i.e. analogous to Formula 1a wherein Z is a direct bond and R¹ is H), which are useful as starting compounds for the methods of Schemes 6 and 7, can be prepared, by condensation of compounds of Formula 2 with carbon suboxide (3f) (see, for example, J. Org. Chem. 1972, 37(9), 1422-1425) as shown in Scheme 8. The reactions are typically performed in an inert solvent such as ether and can include the use of a catalyst such as AlCl₃.

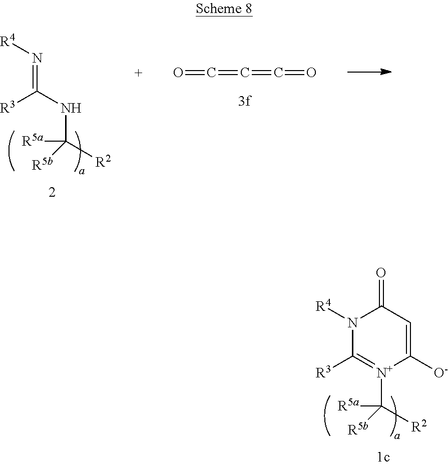

Compounds of Formula 1a wherein Z is a direct bond and R¹ is C₂-C₈ alkenyl or an optionally substituted aromatic ring or ring system, can be prepared from compounds of Formula 1e (i.e. analogous to Formula 1 wherein Z is a direct bond and R¹ is Cl, Br or I, preferably Br or I) and compounds of Formula 8 wherein R¹ is C₂-C₈ alkenyl or an optionally substituted aromatic ring or ring system, and M with Z—R¹ forms a boronic acid, boronic acid ester or trifluoroborate salt, or M is trialkylstannyl or zinc and Z is a direct bond, as shown in Scheme 9.

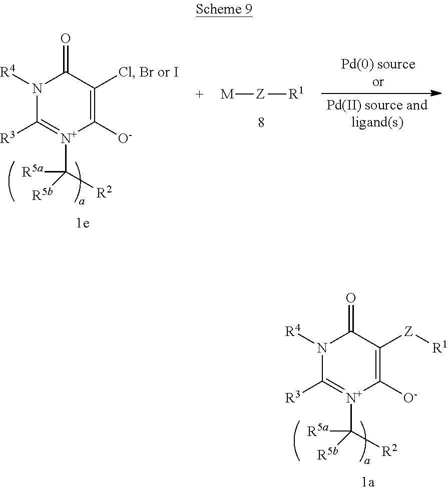

In a similar manner, compounds of Formula 1 wherein a substituent (e.g., R¹ or R²) consists of two directly bonded aromatic rings or ring systems (e.g., a phenyl ring bonded to a second phenyl ring, a phenyl ring bonded to a pyridinyl ring, or a pyridinyl ring bonded to a second pyridinyl ring) can be prepared, by palladium-catalyzed coupling of the two appropriately substituted aromatic rings or ring systems. These palladium-catalyzed couplings between an aromatic chloride, bromide or iodide and an aromatic boronic acid or ester, or an aromatic tin or zinc reagent, are well known and have been extensively described in the art. For example, see Scheme 9a, wherein a compound of Formula 13a or 13b is coupled with an appropriately substituted phenyl ring to provide the biphenyl compound of Formula 13c. M is as defined above for Scheme 9.

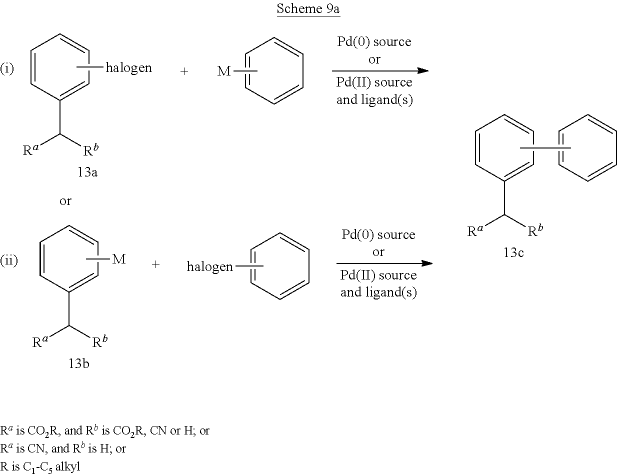

These coupling reactions are typically carried out in the presence of a palladium catalyst and a base optionally under an inert atmosphere. The palladium catalysts used for these coupling reactions typically comprises palladium in a formal oxidation state of either 0 (i.e. Pd(0)) or 2 (i.e. Pd(II)). A wide variety of such palladium-containing compounds and complexes are useful as catalysts for these reactions. Examples of palladium-containing compounds and complexes useful as catalysts in the methods include PdCl₂(PPh₃)₂ (bis(triphenylphosphine)palladium (II) dichloride), Pd(PPh₃)₄ (tetrakis(triphenylphosphine)-palladium(0)), Pd(C₅H₇O₂)₂ (palladium(II) acetylacetonate), Pd₂(dba)₃ (tris(dibenzylidene-acetone)dipalladium(0)), and [1,1′-bis(diphenylphosphino)ferrocene]dichloropalladium(II). These coupling methods are generally conducted, in a liquid phase, and therefore the palladium catalyst preferably has good solubility in the liquid phase. Useful solvents include, for example, water, ethers such as 1,2-dimethoxyethane, amides such as N,N-dimethylacetamide, and non-halogenated aromatic hydrocarbons such as toluene.

The coupling methods can be conducted over a wide range of temperatures, ranging from about 25 to about 200° C. Of note are temperatures from about 60 to about 150° C., which typically provide fast reaction times and high product yields. The general methods and procedures for Stille, Negishi and Suzuki couplings with aryl iodides, bromides or chlorides and an aryl tin, aryl zinc or aryl boronic acid respectively are well known in the literature; see, for example, E. Negishi, Handbook of Organopalladium Chemistry for Organic Synthesis, Wiley-Interscience, 2002, New York, N.Y.

Compounds of Formula 1b wherein R¹ is C₂-C₈ alkynyl can be prepared from compounds of Formula 1e and substituted alkynes of Formula 9 by a Sonogashira coupling reaction as shown in Scheme 10. Sonogashira couplings are well known in the literature. See, for example, K. Sonogashira, Sonogashira Alkyne Synthesis Vol 2, p. 493 in E. Negishi, Handbook of Organopalladium Chemistry for Organic Synthesis, Wiley-Interscience, 2002, New York, N.Y.

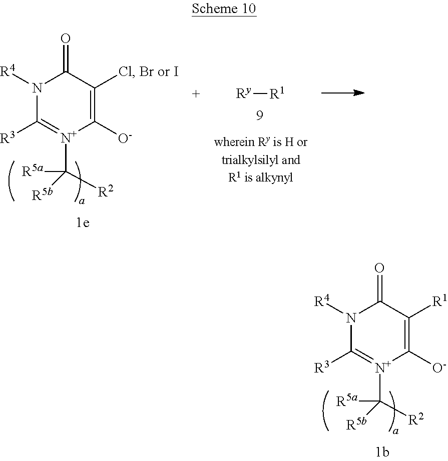

Compounds of Formula 1a wherein Z is O can be prepared by reaction of appropriately substituted alcohols of Formula 10 (e.g., alkyl alcohols or phenols) with compounds of Formula 1e in the presence of a Cu source as shown in Scheme 11. The Ullmann reaction; for example, see Hayashi, S.; Nakanishi, W. Bulletin of the Chemical Society of Japan 2008, 81(12), 1605-1615. This Cu-catalyzed reaction is typically performed at room temperature to 200° C., more typically at 100 to 150° C., and in a solvent such as N,N-dimethylformamide or N-methylpyrrolidinone. Alternatively, this method can be performed in the presence of a Pd source (for example, see Buchwald, S. et al. Angew. Chem. Int. Ed. 2006, 45, 1-7). This Pd-catalyzed reaction is typically performed, at room temperature to 200° C., more typically at 100 to 150° C., and in the presence of a base such as K₃PO₄, and in the presence of a ligand such as 2-di-tert-butylphosphino-2′,4′,6′-triisopropylbiphenyl (i.e. di-t-BuXphos) in an inert solvent such as toluene.

Compounds of Formula 1a wherein Z is NR⁶ can be prepared by reaction of appropriately substituted, amines of Formula 10 (e.g., alkyl amines or anilines) with compounds of Formula 1e in the presence of a Cu source as shown in Scheme 11. The Ullmann reaction; for example, see Xu, H.; Yin, K.; Huang, W. Chemistry—A European Journal 2007, 13(36), 10281-10293. This Cu-catalyzed reaction is typically performed at room temperature to 200° C., more typically at 100 to 150° C., and in a solvent such as N,N-dimethylformamide or N-methylpyrrolidinone. Alternatively, this method can be performed, in the presence of a Pd source (for example, see Uchiyama, M, et al. J. Am. Chem. Soc. 2004, 126(28), 8755-8759). This Pd-catalyzed reaction is typically performed at room temperature to 200° C., more typically at 100 to 150° C., in an inert solvent such as toluene, and in the presence of a base such as NaO-t-Bu.

The method shown in Scheme 11 is demonstrated in Synthesis Example 3 to prepare a compound of this invention where Z is O.

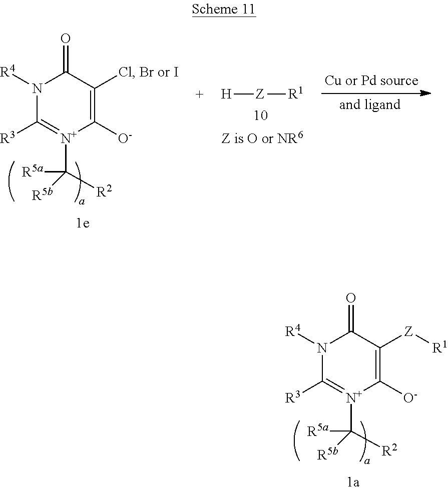

Compounds of Formula 1e can be prepared from compounds of Formula 1c by halogenation using, for example, liquid bromine or N-halosuccinimides (11) as shown in Scheme 12. Typically the reaction is performed in an inert solvent, more typically a halogenated solvent such as methylene chloride or 1,2-dichloroethane. The reaction is typically performed at temperatures from 0 to 80° C., more typically at ambient temperature.

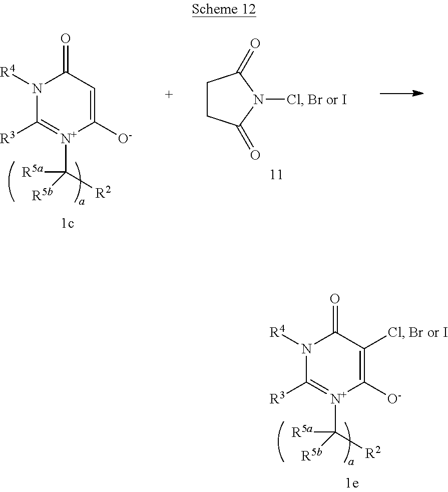

Compounds of Formula 1a can also be prepared, by alkylation of compounds of Formula 12 using appropriately substituted alkylating agents and bases such as potassium carbonate as shown in Scheme 13 (see, for example, Kappe, T. et al. Monatschefte fur Chemie 1971, 102, 412-424 and Urban, M. G.; Arnold, W. Helvetica Chimica Acta 1970, 53, 905-922). Alkylating agents include, but are not limited to, alkyl chlorides, bromides, iodides and sulfonate esters. A wide variety of bases and solvents can be employed in the method of Scheme 13, and these bases and solvents are well known in the art.

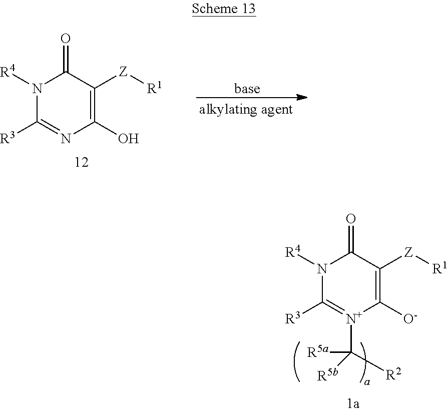

Compounds of Formula 12 can be prepared from compounds of Formula 2a by methods analogous to those shown in Schemes 1 through 4 wherein the compound of Formula 2 is replaced by a compound of Formula 2a. Compounds of Formula 2a are commercially available or can be prepared by general methods well known in the art.

Compounds of Formula 1 wherein X and/or Y are S can be prepared from corresponding compounds of Formula 1a by general methods known in the art involving treatment with thionating reagents such as P₄S₁₀ or Lawessen's Reagent (2,4-bis-(4-methoxyphenyl)-1,3-dithia-2,4-diphosphetane 2,4-disulfide). Alternatively, malonic acids of Formula 3a can be treated with P₂S₆(CH₃)₂ as described in J. Am. Chem. Soc. 1988, 110 (4), 1316-1318. The resulting malonic acid sulfur derivatives can then be used to prepare the compounds of Formula 1 wherein X and/or Y are S by the method of Scheme 1.

Schemes 1 through 13 illustrate methods to prepare compounds of Formula 1 having a variety of substituents noted for R¹, R², R³, R⁴, R^{5a}, R^{5b} and Z. Compounds of Formula 1 having R¹, R², R³, R⁴, R^{5a}, R^{5b} and Z substituents other than those particularly noted for Schemes 1 through 13 can be prepared by general methods known in the art of synthetic organic chemistry, including methods analogous to those described for Schemes 1 to 13.

Examples of intermediates useful in the preparation of compounds of this invention are shown in Tables I-1 through I-39. The following abbreviations are used in the Tables which follow: CN means cyano, Me means methyl, Et means ethyl, Pr means propyl, c-Pr means cyclopropyl, i-Pr means isopropyl, Ph means phenyl, C(O)O(2,4,6-trichlorophenyl) means

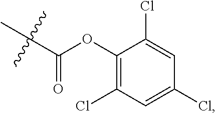

C(O)O(4-nitrophenyl) means

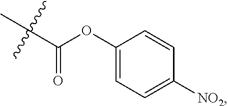

and
C(O)(3-methyl-2 pyridinylamino) means

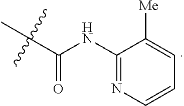

TABLE I-1

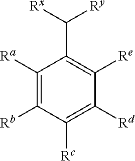

R^{x} is C(O)OH; R^{y} is H; R^{b}, R^{c}, R^{d} and R^{e} are H
R^{a}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a}, R^{c}, R^{d} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a}, R^{b}, R^{d} and R^{e} are H
R^{c}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is F; R^{c}, R^{d} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is F; R^{b}, R^{d} and R^{e} are H
R^{c}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is F; R^{b}, R^{c} and R^{e} are H
R^{d}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is F; R^{b}, R^{c} and R^{d} are H
R^{e}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is Cl; R^{c}, R^{d} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is Cl; R^{b}, R^{d} and R^{e} are H
R^{c}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is Cl; R^{b}, R^{c} and R^{e} are H
R^{d}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is Cl; R^{b}, R^{c} and R^{d} are H
R^{e}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is OMe: R^{c}, R^{d} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is OMe; R^{b}, R^{d} and R^{e} are H
R^{c}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is OMe; R^{b}, R^{c} and R^{e} are H
R^{d}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is OMe; R^{b}, R^{c} and R^{d} are H
R^{c}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is Me; R^{c}, R^{d} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is Me; R^{b}, R^{d} and R^{e} are H
R^{c}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is Me; R^{b}, R^{c} and R^{e} are H
R^{d}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropoylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is Me; R^{b}, R^{c} and R^{d} are H
R^{e}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{d} is Cl; R^{a}, R^{c} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(2-pyridinyl)
OCH₂(6-chloro-3-pyridinyl)
R^{x} is C(O)OH; R^{y} is H; R^{d} is CF₃; R^{a}, R^{c} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{b} is Br; R^{a}, R^{c} and R^{e} are H
R^{d}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{b} is OCF₃; R^{a}, R^{c} and R^{e} are H
R^{d}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{b} is OCH₃; R^{a}, R^{c} and R^{e} are H
R^{d}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} and R^{b} are F; R^{c} and R^{e} are H
R^{d}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} is F; R^{b} is Cl; R^{c} and R^{e} are H
R^{d}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{x} is C(O)OH; R^{y} is H; R^{a} and R^{e} are F; R^{c} and R^{d} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)

### Table I-2

Table I-2 is constructed the same as Table I-1, except that R^{x} is C(O)OMe.

### Table I-3

Table I-3 is constructed the same as Table I-1, except that R^{x} is C(O)OEt.

### Table I-4

Table I-4 is constructed the same as Table I-1, except that R^{x} is C(O)OPh.

### Table I-5

Table I-5 is constructed the same as Table I-1, except that R^{x} is C(O)OC(CH₃)₃.

### Table I-5a

Table I-5a is constructed the same as Table I-1, except that R^{x} is C(O)O(2,4,6-trichlorophenyl).

### Table I-5b

Table I-5b is constructed the same as Table I-1, except that R^{x} is C(O)O(4-nitrophenyl).

### Table I-6

Table I-6 is constructed the same as Table I-1, except that R^{x} is C(O)OH and R^{y} is C(O)OH.

### Table I-7

Table I-7 is constructed the same as Table I-1, except that R^{x} is C(O)OH and R^{y} is C(O)OMe.

### Table I-8

Table I-8 is constructed the same as Table I-1, except that R^{x} is C(O)OH and R^{y} is C(O)OEt.

### Table I-9

Table I-9 is constructed the same as Table I-1, except that R^{x} is C(O)OH and R^{y} is C(O)OC(CH₃)₃.

### Table I-10

Table I-10 is constructed the same as Table I-1, except that R^{x} is C(O)OH and R^{y} is C(O)OPh.

### Table I-10a

Table I-10a is constructed the same as Table I-1, except that R^{x} is C(O)OH and R^{y} is C(O)O(2,4,6-trichlorophenyl).

### Table I-10b

Table I-10b is constructed the same as Table I-1, except that R^{x} is C(O)OH and R^{y} is C(O)O(4-nitrophenyl).

### Table I-11

Table I-11 is constructed the same as Table I-1, except that R^{x} is C(O)OPh and R^{y} is C(O)OMe.

### Table I-12

Table I-12 is constructed the same as Table I-1, except that R^{x} is C(O)OPh and R^{y} is C(O)OEt.

### Table I-13

Table I-13 is constructed the same as Table I-1, except that R^{x} is C(O)OPh and R^{y} is C(O)OC(CH₃)₃.

### Table 1-14

Table I-14 is constructed the same as Table I-1, except that R^{x} is C(O)OPh and R^{y} is C(O)OPh.

### Table I-14a

Table I-14a is constructed the same as Table I-1, except that R^{x} is C(O)OPh and R^{y} is C(O)O(2,4,6-trichlorophenyl).

### Table I-14b

Table I-14b is constructed the same as Table I-1, except that R^{x} is C(O)OPh and R^{y} is C(O)O(4-nitrophenyl).

### Table I-15

Table I-15 is constructed the same as Table I-1, except that R^{x} is C(O)Cl and R^{y} is C(O)Cl.

### Table I-16

Table I-16 is constructed the same as Table I-1, except that R^{x} is C(O)OMe and R^{y} is C(O)OMe.

### Table I-17

Table I-17 is constructed the same as Table I-1, except that R^{x} is C(O)OEt and R^{y} is C(O)OEt.

### Table I-18

Table I-18 is constructed the same as Table I-1, except that R^{x} is C(O)OC(CH₃)₃ and R^{y} is C(O)OC(CH₃)₃.

### Table I-19

Table I-19 is constructed the same as Table I-1, except that R^{x} is C(O)O(2,4,6-trichlorophenyl) and R^{y} is C(O)O(2,4,6-trichlorophenyl).

### Table I-19a

Table I-19a is constructed the same as Table I-1, except that R^{x} is C(O)O(2,4,6-trichlorophenyl) and R^{y} is C(O)O(4-nitrophenyl).

### Table I-20

Table I-20 is constructed the same as Table I-1, except that R^{x} is C(O)(2-pyridinylamino) and R^{y} is C(O)OH.

### Table I-21

Table I-21 is constructed the same as Table I-1, except that R^{x} is C(O)(2-pyridinylamino) and R^{y} is C(O)OMe.

### Table I-22

Table I-22 is constructed the same as Table I-1, except that R^{x} is C(O)(2-pyridinylamino) and R^{y} is C(O)OEt.

### Table I-23

Table I-23 is constructed the same as Table I-1, except that R^{x} is C(O)(2-pyridinylamino) and R^{y} is C(O)OPh.

### Table I-24

Table I-24 is constructed the same as Table I-1, except that R^{x} is C(O)(2-pyridinylamino) and R^{y} is C(O)OC(CH₃)₃.

TABLE I-25

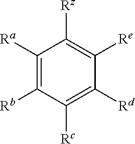

R^{z} is Cl; R^{b}, R^{c}, R^{d} and R^{e} are H
R^{a}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cycloproylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{z} is Cl; R^{a}, R^{c}, R^{d} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C═CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{z} is C; R^{a}, R^{b}, R^{d} and R^{e} are H
R^{c}
—OC≡N
cyclopropyl
2-methylcyclopropyl
2,2-difluorocyclopropyl
CH₂(cyclopropyl)
2,2-dichlorocylcopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{z} is Br; R^{b}, R^{c}, R^{d} and R^{e} are H
R^{a}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{z} is Br; R^{a}, R^{c}, R^{d} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{z} is Br; R^{a}, R^{b}, R^{d} and R^{e} are H
R^{c}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{z} is I; R^{b}, R^{c}, R^{d} and R^{e} are H
R^{a}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{z} is I; R^{a}, R^{c}, R^{d} and R^{e} are H
R^{b}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)
R^{z} is I; R^{a}, R^{b}, R^{d} and R^{e} are H
R^{c}
—OC≡N
cyclopropyl
2-methylcyclopropyl
2,2-difluorocyclopropyl
CH₂(cyclopropyl)
2,2-dichlorocyclopropyl
CH₂OCF₃
CH₂OCHF₂
cyclopropylthio
cyclopropylsulfinyl
cyclopropylsulfonyl
CH₂CN
CH₂CO₂Et
CH═CHCN
C≡CCH₂CN
CO₂CH₂CF₃
OC(O)CF₃
N(Me)C(O)CF₃
OCH₂CH═CH₂
OCH₂C≡CH
cyclopropoxy
OCH₂CN
OCH₂CF₃
C(═NOMe)Me
C(═NOEt)Me
CH₂(2,2-dichlorocyclopropyl)
C(O)N(Me)CH₂CF₃
3-bromo-4,5-dihydro-isoxazol-5-yl
3-methyl-4,5-dihydro-isoxazol-5-yl
OCH₂(2-pyridinyl)
OCH₂(6-chloro-2-pyridinyl)
OCH₂(3-pyridinyl)
OCH₂(6-chloro-3-pyridinyl)

TABLE I-26

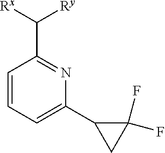

| R^{x} | R^{y} |
|---|---|
| H | C(O)O(2,4,6-trichlorophenyl) |
| H | C(O)O(4-nitrophenyl) |
| C(O)OH | C(O)O(4-nitrophenyl) |
| C(O)OH | C(O)O(2,4,6-tichlorophenyl) |
| C(O)OH | C(O)(3-methyl-2-pyridinylamino) |
| C(O)OMe | C(O)(3-methyl-2-pyridinylamino) |
| C(O)OEt | C(O)(3-methyl-2-pyridinylamino) |
| C(O)OPh | C(O)O(4-nitrophenyl) |
| C(O)OPh | C(O)O(2,4,6-trichlorophenyl) |
| C(O)OPh | C(O)(3-methyl-2-pyridinylamino) |
| C(O)OC(CH | C(O)OPh |
| C(O)OC(CH₃)₃ | C(O)(3-methyl-2-pyridinylamino) |
| C(O)O(4-nitrophenyl) | C(O)O(4-nitrophenyl) |
| C(O)O(4-nitrophenyl) | C(O)(3-methyl-2-pyridinylamino) |
| C(O)O(2,4,64richlorophenyl) | C(O)O(2,4,6-trichlorophenyl) |
| C(O)O(2,4,6-trichlorophenyl) | C(O)(3-methyl-2-pyridinylamino) |
| C(O)OH | C(O)OH |
| C(O)OH | C(O)OMe |
| C(O)OH | C(O)OEt |
| C(O)OH | C(O)OPh |
| C(O)OH | C(O)OC(CH₃)₃ |
| C(O)Cl | C(O)Cl |
| C(O)OMe | C(O)OMe |
| C(O)OEt | C(O)OEt |
| C(O)OPh | C(O)OPh |
| C(O)OC(CH₃)₃ | C(O)OC(CH₃)₃ |
| C(O)OMe | C(O)OPh |
| C(O)OEt | C(O)OPh |
| H | C(O)OH |
| H | C(O)OMe |
| H | C(O)OEt |
| H | C(O)OPh |
| H | C(O)OC(CH₃)₃ |

TABLE I-27

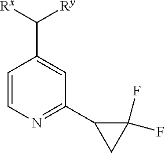

| R^{x} | R^{y} |
|---|---|
| H | C(O)O(2,4,6-trichlorophenyl) |
| H | C(O)O(4-nitrophenyl) |
| C(O)OH | C(O)O(4-nitrophenyl) |
| C(O)OH | C(O)O(2,4,6-trichlorophenyl) |
| C(O)OH | C(O)(3-methyl-2-pyridinylamino) |
| C(O)OMe | C(O)(3-methyl-2-pyridinylamino) |
| C(O)OEt | C(O)(3-methyl-2-pyridinylamino) |
| C(O)OPh | C(O)O(4-nitrophenyl) |
| C(O)OPh | C(O)O(2,4,6-trichlorophenyl) |
| C(O)OPh | C(O)(3-methyl-2-pyridinylamino) |
| C(O)OC(CH₃)₃ | C(O)OPh |
| C(O)OC(CH₃)₃ | C(O)(3-methyl-2-pyridinylamino) |
| C(O)O(4-nitrophenyl) | C(O)O(4-nitrophenyl) |
| C(O)O(4-nitrophenyl) | C(O)(3-methyl-2-pyridinylamino) |
| C(O)O(2,4,6-trichlorophenyl) | C(O)O(2,4,6-trichlorophenyl) |
| C(O)O(2,4,6-trichlorophenyl) | C(O)(3-methyl-2-pyridinylamino) |
| C(O)OH | C(O)OH |
| C(O)OH | C(O)OMe |
| C(O)OH | C(O)OEt |
| C(O)OH | C(O)OPh |
| C(O)OH | C(O)OC(CH₃)₃ |
| C(O)Cl | C(O)Cl |
| C(O)OMe | C(O)OMe |
| C(O)OEt | C(O)OEt |
| C(O)OPh | C(O)OPh |
| C(O)OC(CH₃)₃ | C(O)OC(CH₃)₃ |
| C(O)OMe | C(O)OPh |
| C(O)OEt | C(O)OPh |
| H | C(O)OH |
| H | C(O)OMe |
| H | C(O)OEt |
| H | C(O)OPh |
| H | C(O)OC(CH₃)₃ |

TABLE I-28

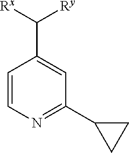

| R^{x} | R^{y} |
|---|---|
| H | C(O)O(2,4,6-trichlorophenyl) |
| H | C(O)O(4-nitrophenyl) |
| C(O)OH | C(O)O(4-nitrophenyl) |
| C(O)OH | C(O)O(2,4,6-trichlorophenyl) |
| C(O)OH | C(O)(3-methyl-2-pyridinylamino) |
| C(O)OMe | C(O)(3-methyl-2-pyridinylamino) |
| C(O)OEt | C(O)(3-methyl-2-pyridinylamino) |
| C(O)OPh | C(O)O(4-nitrophenyl) |
| C(O)OPh | C(O)O(2,4,6-trichlorophenyl) |
| C(O)OPh | C(O)(3-methyl-2-pyridinylamino) |
| C(O)OC(CH₃)₃ | C(O)OPh |
| C(O)OC(CH₃)₃ | C(O)(3-methyl-2-pyridinylamino) |
| C(O)O(4-nitrophenyl) | C(O)O(4-nitrophenyl) |
| C(O)O(4-nitrophenyl) | C(O)(3-methyl-2-pyridinylamino) |
| C(O)O(2,4,6-trichlorophenyl) | C(O)O(2,4,6-trichlorophenyl) |
| C(O)O(2,4,6-trichlorophenyl) | C(O)(3-methyl-2-pyridinylamino) |
| C(O)OH | C(O)OH |
| C(O)OH | C(O)OMe |
| C(O)OH | C(O)OEt |
| C(O)OH | C(O)OPh |
| C(O)OH | C(O)OC(CH₃)₃ |
| C(O)Cl | C(O)Cl |
| C(O)OMe | C(O)OMe |
| C(O)OEt | C(O)OEt |
| C(O)OPh | C(O)OPh |
| C(O)OC(CH₃)₃ | C(O)OC(CH₃)₃ |
| C(O)OMe | C(O)OPh |
| C(O)OEt | C(O)OPh |
| H | C(O)OH |
| H | C(O)OMe |
| H | C(O)OEt |
| H | C(O)OPh |
| H | C(O)OC(CH₃)₃ |

TABLE I-29

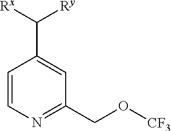

| R^{x} | R^{y} |
|---|---|
| H | C(O)O(2,4,6-trichlorophenyl) |
| H | C(O)O(4-nitrophenyl) |
| C(O)OH | C(O)O(4-nitrophenyl) |
| C(O)OH | C(O)O(2,4,6-trichlorophenyl) |
| C(O)OH | C(O)(3-methyl-2-pyridinylamino) |
| C(O)OMe | C(O)(3-methyl-2-pyridinylamino) |
| C(O)OEt | C(O)(3-methyl-2-pyridinylamino) |
| C(O)OPh | C(O)O(4-nitrophenyl) |
| C(O)OPh | C(O)O(2,4,6-trichlorophenyl) |
| C(O)OPh | C(O)(3-methyl-2-pyridinylamino) |
| C(O)OC(CH₃)₃ | C(O)OPh |
| C(O)OC(CH₃)₃ | C(O)(3-methyl-2-pyridinylamino) |
| C(O)O(4-nitrophenyl) | C(O)O(4-nitrophenyl) |
| C(O)O(4-nitrophenyl) | C(O)(3-methy3-2-pyridinylamino) |
| C(O)O(2,4,6-trichlorophenyl) | C(O)O(2,4,6-trichlorophenyl) |
| C(O)O(2,4,6-trichlorophenyl) | C(O)(3-methyt-2-pyridinylamino) |
| C(O)OH | C(O)OH |
| C(O)OH | C(O)OMe |
| C(O)OH | C(O)OEt |
| C(O)OH | C(O)OPh |
| C(O)OH | C(O)OC(CH₃)₃ |
| C(O)Cl | C(O)Cl |
| C(O)OMe | C(O)OMe |
| C(O)OEt | C(O)OEt |
| C(O)OPh | C(O)OPh |
| C(O)OC(CH₃)₃ | C(O)OC(CH₃)₃ |
| C(O)OMe | C(O)OPh |
| C(O)OEt | C(O)OPh |
| H | C(O)OH |
| H | C(O)OMe |
| H | C(O)OEt |
| H | C(O)OPh |
| H | C(O)OC(CH₃)₃ |

TABLE I-30

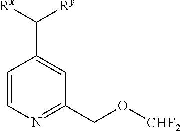

| R^{x} | R^{y} |
|---|---|
| H | C(O)O(2,4,6-trichlorophenyl) |
| H | C(O)O(4-nitrophenyl) |
| C(O)OH | C(O)O(4-nitrophenyl) |
| C(O)OH | C(O)O(2,4,6-trichlorophenyl) |
| C(O)OH | C(O)(3-methyl-2-pyridinylamino) |
| C(O)OMe | C(O)(3-methyl-2-pyridinylamino) |
| C(O)OEt | C(O)(3-methyl-2-pyridinylamino) |
| C(O)OPh | C(O)O(4-nitrophenyl) |
| C(O)OPh | C(O)O(2,4,6-trichlorophenyl) |
| C(O)OPh | C(O)(3-methyl-2-pyridinylamino) |
| C(O)OC(CH₃)₃ | C(O)OPh |
| C(O)OC(CH₃)₃ | C(O)(3-methyl-2-pyridinylamino) |
| C(O)O(4-nitrophenyl) | C(O)O(4-nitrophenyl) |
| C(O)O(4-nitrophenyl) | C(O)(3-methyl-2-pyridinylamino) |
| C(O)O(2,4,6-trichlorophenyl) | C(O)O(2,4,6-trichlorophenyl) |
| C(O)O(2,4,6-trichlorophenyl) | C(O)(3-methyl-2-pyridinylamino) |
| C(O)OH | C(O)OH |
| C(O)OH | C(O)OMe |
| C(O)OH | C(O)OEt |
| C(O)OH | C(O)OPh |
| C(O)OH | C(O)OC(CH₃)₃ |
| C(O)Cl | C(O)Cl |
| C(O)OMe | C(O)OMe |
| C(O)OEt | C(O)OEt |
| C(O)OPh | C(O)OPh |
| C(O)OC(CH₃)₃ | C(O)OC(CH₃)₃ |
| C(O)OMe | C(O)OPh |
| C(O)OEt | C(O)OPh |
| H | C(O)OH |
| H | C(O)OMe |
| H | C(O)OEt |
| H | C(O)OPh |
| H | C(O)OC(CH₃)₃ |

TABLE I-31

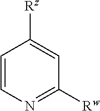

| R^{z} | R^{w} |
|---|---|
| Cl | 2,2-difluorocycloprop-1-yl |
| Cl | cyclopropyl |
| Cl | CH₂OCF₃ |
| Cl | CH₂OCHF₂ |
| Br | 2,2-difluorocycloprop-1-yl |
| Br | cyclopropyl |
| Br | CH₂OCF₃ |
| Br | CH₂OCHF₂ |
| I | 2,2-difluorocycloprop-1-yl |
| I | cyclopropyl |
| I | CH₂OCF₃ |
| I | CH₂OCHF₂ |
| NH₂ | 2,2-difluorocycloprop-1-yl |
| NH₂ | cyclopropyl |
| NH₂ | CH₂OCF₃ |
| NH₂ | CH₂OOCHF₂ |
| CH₃ | 2,2-difluorocycloprop-1-yl |
| CH₃ | cyclopropyl |
| CH₃ | CH₂OCF₃ |
| CH₃ | CH₂OCHF₂ |
| CH₂CN | 2,2-difluorocycloprop-1-yl |
| CH₂CN | cyclopropyl |
| CH₂CN | CH₂OCF₃ |
| CH₂CN | CH₂OCHF₂ |
| CH₂Cl | 2,2-difluorocycloprop-1-yl |
| CH₂Cl | cyclopropyl |
| CH₂Cl | CH₂OCF₃ |
| CH₂Cl | CH₂OCHF₂ |

TABLE I-32

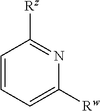

| R^{z} | R^{w} |
|---|---|
| Cl | 2,2-difluorocycloprop-1-yl |
| Br | 2,2-difluorocycloprop-1-yl |
| I | 2,2-difluorocycloprop-1-yl |
| NH₂ | 2,2-difluorocycloprop-1-yl |
| CH₃ | 2,2-difluorocycloprop-1-yl |
| CH₂CN | 2,2-difluorocycloprop-1-yl |
| CH₂Cl | 2,2-difluorocycloprop-1-yl |

TABLE I-33

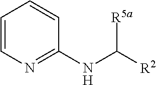

| R^{5a} | R² |
|---|---|
| H | CF₃ |
| Me | CF₃ |
| H | Et |
| Me | Et |
| H | 3-pyridinyl |
| Me | 3-pyridinyl |
| H | 6-fluoro-3-pyridinyl |
| Me | 6-fluoro-3-pyridinyl |
| H | 6-brotno-3-pyridinyl |
| Me | 6-bromo-3-pyridinyl |
| H | 2-methyl-5-thiazolyl |
| Me | 2-methyl-5-thiazolyl |
| H | 2-chloro-5-thiazolyl |
| Me | 2-chloro-5-thiazolyl |
| H | 1-methyl-4-pyrazolyl |
| Me | 1-methyl-4-pyrazolyl |
| H | 5-pyrimidinyl |
| Me | 5-pyrimidinyl |
| H | CH₂CHFCF₂Cl |
| Me | CH₂CHFCF₂Cl |
| H | cyclopropyl |
| Me | cyclopropyl |
| H | 6-methyl-3-pyridinyl |
| Me | 6-methyl-3-pyridinyl |
| H | 6-chloro-3-pyridinyl |
| Me | 6-chloro-3-pyridinyl |
| H | 5-thiazolyl |
| Me | 5-thiazolyl |
| H | 2-fluoro-5-thiazotyl |
| Me | 2-fluoro-5-thiazolyl |
| H | 2-brorno-5-thiazolyl |
| Me | 2-bromo-5-thiazolyl |
| H | 3-methyl-5-isoxazolyl |
| Me | 3-methyl-5-isoxazolyl |
| H | 2-methyl-5-pyrimidinyl |
| Me | 2-methyl-5-pyrimidinyl |

TABLE I-34

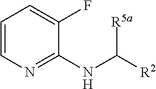

| R^{5a} | R² |
|---|---|
| H | CF₃ |
| Me | CF₃ |
| H | Et |
| Me | Et |
| H | 3-pyridinyl |
| Me | 3-pyridinyl |
| H | 6-fluoro-3-pyridinyl |
| Me | 6-fluoro-3-pyridinyl |
| H | 6-bromo-3-pyridinyl |
| Me | 6-bromo-3-pyridinyl |
| H | 2-methyl-5-thiazolyl |
| Me | 2-methyl-5-thiazolyl |
| H | 2-chloro-5-thiazolyl |
| Me | 2-chloro-5-thiazolyl |
| H | 1-methyl-4-pyrazolyl |
| Me | 1-methyl-4-pyrazolyl |
| H | 5-pyrimidinyl |
| Me | 5-pyrimidinyl |
| H | CH₂CHFCF₂Cl |
| Me | CH₂CHFCF₂Cl |
| H | cyclopropyl |
| Me | cyclopropyl |
| H | 6-methyl-3-pyridinyl |
| Me | 6-methyl-3-pyridinyl |
| H | 6-chloro-3-pyridinyl |
| Me | 6-chloro-3-pyridinyl |
| H | 5-thiazolyl |
| Me | 5-thiazolyl |
| H | 2-fluoro-5-thiazolyl |
| Me | 2-fluoro-5-thiazolyl |
| H | 2-bromo-5-thiazolyl |
| Me | 2-bromo-5-thiazolyl |
| H | 3-methyl-5-isoxazolyl |
| Me | 3-methyl-5-isoxazolyl |
| H | 2-methyl-5-pyrimidinyl |
| Me | 2-methyl-5-pyrimidinyl |

TABLE I-35

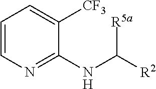

| R^{5a} | R² |
|---|---|
| H | CF₃ |
| Me | CF₃ |
| H | Et |
| Me | Et |
| H | 3-pyridinyl |
| Me | 3-pyridinyl |
| H | 6-fluoro-3-pyridinyl |
| Me | 6-fluoro-3-pyridinyl |
| H | 6-bromo-3-pyridinyl |
| Me | 6-bromo-3-pyridinyl |
| H | 2-methyl-5-thiazolyl |
| Me | 2-methyl-5-thiazolyl |
| H | 2-chloro-5-thiazolyl |
| Me | 2-chloro-5-thiazolyl |
| H | 1-methyl-4-pyrazolyl |
| Me | 1-methyl-4-pyrazolyl |
| H | 5-pyrimidinyl |
| Me | 5-pyrimidinyl |
| H | CH₂CHFCF₂Cl |
| Me | CH₂CHFCF₂Cl |
| H | cyclopropyl |
| Me | cyclopropyl |
| H | 6-methyl-3-pyridinyl |
| Me | 6-methyl-3-pyridinyl |
| H | 6-chloro-3-pyridinyl |
| Me | 6-chloro-3-pyridinyl |
| H | 5-thiazolyl |
| Me | 5-thiazolyl |
| H | 2-fluoro-5-thiazolyl |
| Me | 2-fluoro-5-thiazolyl |
| H | 2-bromo-5-thiazolyl |
| Me | 2-bromo-5-thiazolyl |
| H | 3-methyl-5-isoxazolyl |
| Me | 3-methyl-5-isoxazolyl |
| H | 2-methyl-5-pyrimidinyl |
| Me | 2-methyl-5-pyrimidinyl |

TABLE I-36

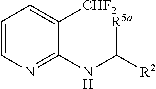

| R^{5a} | R² |
|---|---|
| H | CF₃ |
| Me | CF₃ |
| H | Et |
| Me | Et |
| H | 3-pyridinyl |
| Me | 3-pyridinyl |
| H | 6-fluoro-3-pyridinyl |
| Me | 6-fluoro-3-pyridinyl |
| H | 6-bromo-3-pyridinyl |
| Me | 6-bromo-3-pyridinyl |
| H | 2-methyl-5-thiazolyl |
| Me | 2-methyl-5-thiazolyl |
| H | 2-chloro-5-thiazolyl |
| Me | 2-chloro-5-thiazolyl |
| H | 1-methyl-4-pyrazolyl |
| Me | 1-methyl-4-pyrazolyl |
| H | 5-pyrimidinyl |
| Me | 5-pyrimidinyl |
| H | CH₂CHFCF₂Cl |
| Me | CH₂CHFCF₂Cl |
| H | cyclopropyl |
| Me | cyclopropyl |
| H | 6-methyl-3-pyridinyl |
| Me | 6-methyl-3-pyridinyl |
| H | 6-chloro-3-pyridinyl |
| Me | 6-chloro-3-pyridinyl |
| H | 5-thiazolyl |
| Me | 5-thiazolyl |
| H | 2-fluoro-5-thiazolyl |
| Me | 2-fluoro-5-thiazolyl |
| H | 2-bromo-5-thiazolyl |
| Me | 2-bromo-5-thiazolyl |
| H | 3-methyl-5-isoxazolyl |
| Me | 3-methyl-5-isoxazolyl |
| H | 2-methyl-5-pyrimidinyl |
| Me | 2-methyl-5-pyrimidinyl |

TABLE I-37

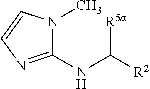

| R^{5a} | R² |
|---|---|
| H | CF₃ |
| Me | CF₃ |
| H | Et |
| Me | Et |
| H | 3-pyridinyl |
| Me | 3-pyridinyl |
| H | 6-fluoro-3-pyridinyl |
| Me | 6-fluoro-3-pyridinyl |
| H | 6-bromo-3-pyridinyl |
| Me | 6-brorno-3-pyridinyl |
| H | 2-methyl-5-thiazelyl |
| Me | 2-methyl-5-thiazolyl |
| H | 2-chloro-5-thiazolyl |
| Me | 2-chloro-5-thiazolyl |
| H | 1-methyl-4-pyrazolyl |
| Me | 1-methyl-4-pyrazolyl |
| H | 5-pyrimidinyl |
| Me | 5-pyrimidinyl |
| H | CH₂CHFCF₂Cl |
| Me | CH₂CHFCF₂Cl |
| H | cyclopropyl |
| Me | cyclopropyl |
| H | 6-methyl-3-pyridinyl |
| Me | 6-methyl-3-pyridinyl |
| H | 6-chloro-3-pyridinyl |
| Me | 6-chloro-3-pyridinyl |
| H | 5-thiazolyl |
| Me | 5-thiazolyl |
| H | 2-fluoro-5-thiazolyl |
| Me | 2-fluoro-5-thiazolyl |
| H | 2-bromo-5-thiazolyl |
| Me | 2-bromo-5-thiazolyl |
| H | 3-methyl-5-isoxazolyl |
| Me | 3-methyl-5-isoxazolyl |
| H | 2-methyl-5-pyrimidinyl |
| Me | 2-methyl-5-pyrimidinyl |

TABLE I-38

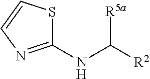

| R^{5a} | R² |
|---|---|
| H | CF₃ |
| Me | CF₃ |
| H | Et |
| Me | Et |
| H | 3-pyridinyl |
| Me | 3-pyridinyl |
| H | 6-fluoro-3-pyridinyl |
| Me | 6-fluoro-3-pyridinyl |
| H | 6-bromo-3-pyridinyl |
| Me | 6-bromo-3-pyridinyl |
| H | 2-methyl-5-thiazolyl |
| Me | 2-methyl-5-thiazolyl |
| H | 2-chloro-5-thiazolyl |
| Me | 2-chloro-5-thiazolyl |
| H | 1-methyl-4-pyrazolyl |
| Me | 1-methyl-4-pyrazolyl |
| H | 5-pyrimidinyl |
| Me | 5-pyrimidinyl |
| H | CH₂CHFCF₂Cl |
| Me | CH₂CHFCF₂Cl |
| H | cyclopropyl |
| Me | cyclopropyl |
| H | 6-methyl-3-pyridinyl |
| Me | 6-methyl -3-pyridinyl |
| H | 6-Chloro-3-pyridinyl |
| Me | 6-chloro-3-pyridinyl |
| H | 5-thiazolyl |
| Me | 5-thiazolyl |
| H | 2-fluoro-5-thiazolyl |
| Me | 2-fluoro-5-thiazolyl |
| H | 2-bromo-5-thiazolyl |
| Me | 2-bromo-5-thiazolyl |
| H | 3-methyl-5-isoxazolyl |
| Me | 3-methyl-5-isoxazolyl |
| H | 2-methyl-5-pyrimidinyl |
| Me | 2-methyl-5-pyrimidinyl |

TABLE I-39

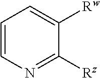

| R^{z} | R^{w} | R^{z} | R^{w} |
|---|---|---|---|
| F | H | F | CF₃ |
| Cl | H | Cl | CF₃ |
| Br | H | Br | CF₃ |
| I | H | I | CF₃ |
| NH₂ | H | NH₂ | CF₃ |
| NHC(O)CH₃ | H | NHC(O)CH₃ | CF₃ |
| NHCHO | H | NHCHO | CF₃ |
| NHC(O)OC(CH₃)₃ | H | NHC(O)OC(CH₃)₃ | CF₃ |
| NHC(O)OCH₂Ph | H | NHC(O)OCH₂Ph | CF₃ |
| F | F | F | CHF₂ |
| Cl | F | Cl | CHF₂ |
| Br | F | Br | CHF₂ |
| I | F | I | CHF₂ |
| NH₂ | F | NH₂ | CHF₂ |
| NHC(O)CH₃ | F | NHC(O)CH₃ | CHF₂ |
| NHCHO | F | NHCHO | CHF₂ |
| NHC(O)OC(CH₃)₃ | F | NHC(O)OC(CH₃)₃ | CHF₂ |
| NHC(O)OCH₂Ph | F | NHC(O)OCH₂Ph | CHF₂ |

It is recognized that some reagents and reaction conditions described, above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled, in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Synthesis Examples are, therefore, to be construed as merely illustrative, and not limiting of the disclosure in any way whatsoever. Steps in the following Synthesis Examples illustrate a procedure for each step in an overall synthetic transformation, and the starting material for each step may not have necessarily been prepared by a particular preparative run whose procedure is described in other Examples or Steps. Ambient or room temperature is defined as about 20-25° C. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “dd” means doublet of doublets, “ddd” means doublet of doublet of doublets, “t” means triplet, “m” means multiplet, and “br s” means broad singlet. For mass spectral data, the numerical value reported is the molecular weight of the parent molecular ion (M) formed by addition of H⁺ (molecular weight of 1) to the molecule to give a M+1 peak observed by mass spectrometry using atmospheric pressure chemical ionization (AP⁺).

### Synthesis Example 1

### Preparation of 3-[3-(cyanomethoxy)phenyl]-2-hydroxy-4-oxo-1-(2,2,2-trifluoroethyl)-4H-pyrido[1,2-a]pyrimidinium inner salt (Compound 37)

### Step A: Preparation of N-(2,2,2-trifluoroethyl)-2-pyridinamine

A mixture of 2-fluoropyridine (2.00 g, 20.6 mmol) and 2,2,2-trifluoroethylamine hydrogen chloride (5.00 g, 36.9 mmol) was heated to 220° C. for 30 min in a microwave reactor. The same reaction was repeated 5 times. The reaction mixtures from all 6 reactions were cooled, combined and diluted with ethyl acetate (150 mL). The organic mixture was neutralized by washing with saturated aqueous sodium bicarbonate, water (30 mL) and brine (30 mL). The organic phase was dried over Na₂SO₄ and concentrated, and the resulting residue was purified by chromatography on silica gel using 80% ethyl acetate/hexane as eiuant to give the title compound as a white solid (17.0 g).

¹H NMR (CDCl₃) δ 8.15 (d, 1H), 7.45 (dd, 1H), 6.69 (dd, 1H), 6.49 (d, 1H), 4.58 (br s, 1H), 4.11 (q, 2H).

### Step B: Preparation of 2-hydroxy-4-oxo-1-(2,2,2-trifluoroethyl)-4H-pyrido[1,2-a]-pyrimidinium inner salt

A solution of dicyclohexylcarbodiimide (1.0 M in dichloromethane, 108 mL, 108 mmol) was added to a solution of N-(2,2,2-trifluoroethyl)-2-pyridinamine (i.e. the product of Step A) (9.51 g, 54.0 mmol) and malonic acid (5.62 g, 54.0 mmol) in dichloromethane (190 mL). The reaction mixture was stirred at room temperature for 24 h. The reaction mixture was then filtered through a pad of Celite® diatomaceous filter aid, and the filtration cake was washed with dichloromethane. The combined organic phases were concentrated under reduced pressure, and the resulting residue was purified by chromatography on silica gel using 50-100% ethyl acetate/hexane as eluant to give the title compound as a pale yellow solid (7.0 g).

¹H NMR (CD₃S(O)CD₃) δ 9.22 (d, 1H), 8.42 (t, 1H), 8.11 (d, 1H), 7.59 (t, 1H), 5.25 (q, 2H), 4.96 (s, 1H).

### Step C: Preparation of 2-hydroxy-4-oxo-3-iodo-1-(2,2,2-trifluoroethyl)-4H-pyrido[1,2-a]-pyrimidinium inner salt

N-iodosuccinimide (1.3 g, 5.7 mmol) was added to a solution of 2-hydroxy-4-oxo-1-(2,2,2-trifluoroethyl)-4H-pyrido[1,2-a]pyrimidminium inner salt (i.e. the product of Step B) (1.4 g, 5.7 mmol) in N,N-dimethylformamide (10 mL) and stirred for 2 min. Water was added, along with sodium thiosulfate, and the mixture was extracted with ethyl acetate three times. The combined organic phases were dried over MgSO₄, and concentrated under reduced pressure. The resulting crude product (1.9 g) was used in the next step without farther purification.

¹H NMR (CDCl₃) δ 9.55 (dd, 1H), 8.25 (m, 1H), 7.6 (dd, 1H), 7.5 (m, 1H), 5.2-5.0 (br s, 2H).

### Step D: Preparation of 3-[3-(cyanomethoxy)phenyl]-2-hydroxy-4-oxo-1-(2,2,2-trifluoroethyl)-4H-pyrido[1,2-a]pyrimidinium inner salt

2-Hydroxy-4-oxo-3-iodo-1-(2,2,2-trifluoroethyl)-4H-pyrido[1,2-a]pyrimidinium inner salt (i.e. the product of Step C) (0.3 g, 0.81 mmol) was combined in a microwave vial with 3-cyanomethoxyphenylboronic acid, pinacol ester (0.331 g, 1.21 mmol), sodium bicarbonate (0.171 g, 1.62 mmol), dichlorobis(triphenylphosphine)palladium(II) (0.028 g, 0.04 mmol), dioxane (3 mL) and water (1 mL) and then heated at 150° C. for 15 min. The reaction mixture was filtered, through an ChemElute® diatomaceous earth-based liquid-liquid, exchange cartridge, and then subjected to chromatography on silica gel using 100% ethyl acetate as eluant to give the title compound, a compound of the present invention, as a yellow solid (73 mg)

¹H NMR (CD₃S(O)CD₃) δ 9.5 (dd, 1H), 8.45 (m, 1H), 8.15 (dd, 1H), 7.7 (m, 1H), 7.64 (m, 2H), 7.3 (m, 1H), 6.9 (m, 1H), 5.45-5.3 (m, 2H), 5.06 (s, 2H).

### Synthesis Example 2

### Preparation of 1-[(2-chloro-5-thiazolyl)methyl]-3-[3-(4,5-dihydro-3-methyl-5-isoxazolyl)phenyl]-2-hydroxy-4-oxo-4H-pyrido[1,2-a]pyrimidinium inner salt (Compound 88)

### Step A: Preparation of 2-(3-ethenylphenyl)propanedioic acid

Diethyl 3-ethenylphenylmalonate (0.4 g) was stirred in an aqueous sodium hydroxide solution (1 mL, 20% by weight) at 60° C. for 1 min. The reaction mixture was then cooled in an ice bath and quenched by addition of 6 N hydrochloric acid to adjust the pH to about 2. The aqueous mixture was extracted with ethyl acetate three times. The combined organic phases were dried (MgSO₄) and concentrated to give the title compound as a white solid (0.215 g).

¹H NMR (CD₃C(O)CD₃) δ 7.59 (s, 1H), 7.45-7.3 (m, 3H), 6.8 (m, 1H), 5.85 (d, 1H), 5.25 (d, 1H), 4.78 (s, 1H).

### Step B: Preparation of 1-[(2-chloro-5-thiazolyl)methyl]-3-(3-ethenylphenyl)-2-hydroxy-4-oxo-4H-pyrido[1,2-a]pyrimidinium inner salt

Oxalyl chloride (0.45 mL, 5.2 mmol) was added at ambient temperature to a slurry of 2-(3-ethenylphenyl)propanedioic acid (i.e. the product of Step A) (0.215 g, 1.04 mmol) in dichloromethane (8 mL). After a catalytic amount of N,N-dimethylformamide was added, gas evolution began. The reaction mixture was stirred for an additional 30 min, during which time gas evolution ceased. The reaction mixture was briefly concentrated under vacuum at 35° C. The resultant oil was taken up in dichloromethane (2 mL) and added to a solution of N-[(chloro-5-thiazolyl)methyl]2-pyridinamine (0.234 g, 1.04 mmol) and triethylamine (0.29 mL, 2.08 mmol) in dichloromethane (10 mL) at 0° C. After stirring for 15 min, the reaction mixture was concentrated, and the resultant residue was purified by chromatography on silica gel using a gradient of ethyl acetate to 5% methanol in ethyl acetate as eluant to give the title compound as a yellow solid (0.117 g).

¹H NMR (CD₃C(O)CD₃) δ 9.45 (d, 1H), 8.35 (m, 1H), 8.15 (m, 1H), 7.97 (d, 1H), 7.95 (d, 1H), 7.75 (m, 1H), 7.55 (m, 1H), 7.28 (m, 2H), 6.75 (m, 1H), 5.74 (m, 3H), 5.2 (d, 1H).

### Step C: Preparation of 1-[(2-chloro-5-thiazolyl)methyl]-3-[3-(4,5-dihydro-3-methyl-5-isoxazolyl)phenyl]-2-hydroxy-4-oxo-4H-pyrido[1,2-a]pyrimidinium inner salt

1-[(2-Chloro-5-thiazoyl)methyl]-3-(3-ethenylphenyl)-2-hydroxy-4-oxo-4H-pyrido[1,2-a]pyrimidinium inner salt (i.e. the product of Step B) (40 mg, 0.1 mmol) was combined in dichloromethane (3 mL) with di-tert-butyl dicarbonate (55 mg, 0.25 mmol) and DMAP (1.4 mg, 0.012 mmol). Nitroethane (0.02 mL, 0.3 mmol) was added as a solution in of dichloromethane (0.5 mL) over 10 min. The reaction mixture was stirred at ambient temperature over 48 h, at which time additional nitroethane (0.02 mL, 0.3 mmol) was added, and then the reaction stirred an additional 24 h. The reaction mixture was evaporated to dryness, then redissolved in dichloromethane and chromatographed on silica gel using a gradient of ethyl acetate to 10% methanol in ethyl acetate as eluent to yield the title compound, a compound of the present invention, as a yellow solid (20 mg).

¹H NMR (CD₃C(O)CD₃) δ 9.45 (dd, 1H), 8.4 (m, 1H), 8.15 (m, 1H), 7.95 (s, 1H), 7.85 (s, 1H), 7.8 (dd, 1H), 7.55 (m, 1H), 7.3 (m, 1H), 7.15 (dd, 1H), 5.75 (br s, 2H), 5.5 (m, 1H), 3.45 (m, 1H), 2.95 (m, 1H), 2.0 (s, 3H).

### Synthesis Example 3

### Preparation of 2-hydroxy-4-oxo-3-phenoxy-1-propyl-4H-pyrido[1,2-a]pyrimidinium inner salt (Compound 4)

### Step A: Preparation of 3-bromo-2-hydroxy-4-oxo-1-propyl-4H-pyrido[1,2-a]pyrimidinium inner salt

2-Hydroxy-4-oxo-1-propyl-4H-pyrido[1,2-a]pyrimidinium inner salt (2.04 g, 10 mmol) was dissolved in ethanol-free chloroform (35 mL), and bromine (613 μL, 12 mmol) was added dropwise. The reaction mixture was stirred for 3 h at ambient temperature and then treated with saturated aqueous sodium bicarbonate. The organic phase wras washed with brine, dried over sodium sulfate and then concentrated, and the resulting residue was triturated with ether to give the title compound.

¹H NMR (CDCl₃) δ 8.47 (dd, 1H), 8.13 (dd, 1H), 7.49 (d, 1H), 7.36 (t, 1H), 4.32 (t, 2H), 1.80 (m, 2H), 1.80 (t, 3H).

### Step B: Preparation of 2-hydroxy-4-oxo-3-phenoxy-1-propyl-4H-pyrido[1,2-a]pyrimidinium inner salt

Phenol (188 mg, 2 mmol) was dissolved in a potassium tertiary butoxide solution (2 ml of 1 N in THF, 2 mmol) and then concentrated. The potassium salt of phenol was dissolved in toluene, concentrated and then dissolved in 1,2-dimethoxyethane (5 mL). 3-Bromo-2-hydroxy-4-oxo-1-propyl-4H-pyrido[1,2-a]pyrimidinium inner salt (i.e. the product of Step A) (285 mg, 1 mmol), copper (I) hexafluorophosphate-tetrakis-acetonitrile (16 mg, 0.05 mmol) and 8-quinolinol (15 mg, 0.1 mmol) were added and the mixture was heated to reflux overnight. The reaction mixture was poured into 3% aqueous ammonium hydroxide and extracted with ethyl acetate. The organic phase was washed with brine, dried and concentrated. The crude product was purified by chromatography on silica gel using 10% ethyl acetate in hexanes as eluent to give the title compound, a compound of the present invention, as a solid (45 mg).

¹H NMR (CDCl₃) δ 9.44 (t, 1H), 8.13 (dd, 1H), 7.50 (dd, 2H), 7.36 (q, 2H), 7.24 (t, 2H), 7.04 (d, 1H), 6.96 (t, 1H), 4.32 (t, 2H), 1.79 (m, 2H), 1.06 (t, 3H).

By the procedures described herein together with methods known in the art, the following compounds of Tables 1 to 29 can be prepared. The following abbreviations are used in the Tables which follow: CN means cyano, Me means methyl, Et means ethyl, Pr means propyl, c-Pr means cyclopropyl, i-Pr means isopropyl and Ph means phenyl.

Tables 1-15 pertain to the structure of Formula T-1 shown below.

TABLE I
T-1

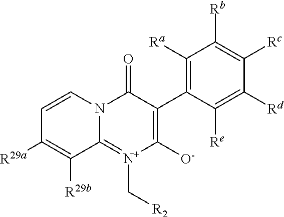

R^{b}, R^{c}, R^{d}, R^{e,} R^{29a} and R^{29b} are H; R² is 2-chloro-5-thiazolyl

| R^{a} | R^{a} | R^{a} | R^{a} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a}, R^{c}, R^{d}, R^{e}, R^{29a} and R^{29b} are H; R² is 2-chloro-5-thiazolyl

| R^{b} | R^{b} | R^{b} | R^{b} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a}, R^{b}, R^{d}, R^{e}, R^{29a} and R^{29b} are H; R² is 2-chloro-5-thiazolyl

| R^{c} | R^{c} | R^{c} | R^{c} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is F; R^{c}, R^{d}, R^{e}, R^{29a} and R^{29b} are H; R² is 2-chloro-5-thiazolyl

| R^{b} | R^{b} | R^{b} | R^{b} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is F; R^{b}, R^{d}, R^{e}, R^{29a} and R^{29b} are H; R² is 2-chloro-5-thiazolyl

| R^{c} | R^{c} | R^{c} | R^{c} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is F; R^{b}, R^{c}, R^{e}, R^{29a} and R^{29b} are H; R² is 2-chloro-5-thiazolyl

| R^{d} | R^{d} | R^{d} | R^{d} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is F; R^{b}, R^{c}, R^{d}, R^{29a} and R^{29b} are H; R² is 2-chloro-5-thiazolyl

| R^{e} | R^{e} | R^{e} | R^{e} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is Cl; R^{c}, R^{d}, R^{e}, R^{29a} and R^{29b} are H; R² is 2-chloro-5-thiazolyl

| R^{b} | R^{b} | R^{b} | R^{b} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is Cl; R^{b}, R^{d}, R^{e}, R^{29a} and R^{29b} are H; R² is 2-chloro-5-thiazolyl

| R^{c} | R^{c} | R^{c} | R^{c} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is Cl; R^{b}, R^{c,} R^{e}, R^{29a} and R^{29b} are H; R² is 2-chloro-5-thiazolyl

| R^{d} | R^{d} | R^{d} | R^{d} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is Cl; R^{b}, R^{c}, R^{d}, R^{29a} and R^{29b} are H; R² is 2-chloro-5-thiazolyl

| R^{e} | R^{e} | R^{e} | R^{e} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is OMe; R^{c}, R^{d}, R^{e}, R^{29a} and R^{29b} are H; R² is 2-chloro-5-thiazolyl

| R^{b} | R^{b} | R^{b} | R^{b} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is OMe; R^{b}, R^{d}, R^{e}, R^{29a} and R^{29b} are H; R² is 2-chloro-5-thiazolyl

| R^{c} | R^{c} | R^{c} | R^{c} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is OMe; R^{b}, R^{c}, R^{e}, R^{29a} and R^{29b} are H; R² is 2-chloro-5-thiazolyl

| R^{d} | R^{d} | R^{d} | R^{d} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is OMe; R^{b}, R^{c}, R^{d}, R^{29a} and R^{29b} are H; R² is 2-chloro-5-thiazolyl

| R^{e} | R^{e} | R^{e} | R^{e} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is Me; R^{c}, R^{d}, R^{e} R^{29a} and R^{29b} are H; R² is 2-chloro-5-thiazolyl

| R^{b} | R^{b} | R^{b} | R^{b} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is Me; R^{b}, R^{d}, R^{e}, R^{29a} and R^{29b} are H; R² is 2-chloro-5-thiazolyl

| R^{c} | R^{c} | R^{c} | R^{c} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is Me; R^{b}, R^{c}, R^{e}, R^{29a} and R^{29b} are H; R² is 2-chloro-5-thiazolyl

| R^{d} | R^{d} | R^{d} | R^{d} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is Me; R^{b}, R^{c}, R^{d}, R^{29a} and R^{29b} are H; R² is 2-chloro-5-thiazolyl

| R^{e} | R^{e} | R^{e} | R^{e} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{d} is Cl; R^{a}, R^{c}, R^{e}, R^{29a} and R^{29b} are H; R² is 2-chloro-5-thiazolyl

| R^{b} | R^{b} | R^{b} | R^{b} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{d} is CF₃; R^{a}, R^{c}, R^{e}, R^{29a} and R^{29b} are H; R² is 2-chloro-5-thiazolyl

| R^{b} | R^{b} | R^{b} | R^{b} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{b} is Br; R^{a}, R^{c}, R^{e}, R^{29a} and R^{29b} are H; R² is 2-chloro-5-thiazolyl

| R^{d} | R^{d} | R^{d} | R^{d} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{b} is OCF₃; R^{a}, R^{c}, R^{e}, R^{29a} and R^{29b} are H; R² is 2-chloro-5-thiazolyl

| R^{d} | R^{d} | R^{d} | R^{d} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{b} is OCH₃; R^{a}, R^{c}, R^{e}, R^{29a} and R^{29b} are H; R² is 2-chloro-5-thiazolyl

| R^{d} | R^{d} | R^{d} | R^{d} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} and R^{b} are F; R^{c}, R^{e}, R^{29a} and R^{29b} are H; R² is 2-chloro-5-thiazolyl

| R^{d} | R^{d} | R^{d} | R^{d} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} is F; R^{b} is Cl, R^{c}, R^{e}, R^{29a} and R^{29b} are H; R² is 2-chloro-5-thiazolyl

| R^{d} | R^{d} | R^{d} | R^{d} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

R^{a} and R^{e} are F; R^{c}, R^{d}, R^{29a} and R^{29b} are H; R² is 2-chloro-5-thiazolyl

| R^{b} | R^{b} | R^{b} | R^{b} |
|---|---|---|---|
| —OC≡N | cyclopropylthio | OC(O)CF₃ | CH₂(2,2-dichlorocyclopropyl) |
| cyclopropyl | cyclopropylsulfinyl | N(Me)C(O)CF₃ | C(O)N(Me)CH₂CF₃ |
| 2-methylcyclopropyl | cyclopropylsulfonyl | OCH₂CH═CH₂ | 3-bromo-4,5-dihydro-isoxazol-5-yl |
| 2,2-difluorocyclopropyl | CH₂CN | OCH₂C≡CH | 3-methyl-4,5-dihydro-isoxazol-5-yl |
| CH₂(cyclopropyl) | CH₂CO₂Et | cyclopropoxy | OCH₂(2-pyridinyl) |
| 2,2-dichlorocyclopropyl | CH═CHCN | OCH₂CN | OCH₂(6-chloro-2-pyridinyl) |
| CH₂OCF₃ | C≡CCH₂CN | OCH₂CF₃ | OCH₂(3-pyridinyl) |
| CH₂OCHF₂ | CO₂CH₂CF₃ | C(═NOMe)Me | OCH₂(6-chloro-3-pyridinyl) |
| | | C(═NOEt)Me | |

### Table 2

Table 2 is constructed the same as Table 1, except that R^{29b} is F. For example, the first compound in Table 2 is the compound, of Formula T-1 wherein R^{a} is —OCN; R^{b}, R^{c}, R^{d}, R^{e} and R^{29a} are H; R^{29b} is F; and R² is 2-chloro-5-thiazolyl.

### Table 3

Table 3 is constructed the same as Table 1, except that R^{29b} is CF₃. For example, the first compound, in Table 3 is the compound of Formula T-1 wherein R^{a} is —OCN; R^{b}, R^{c}, R^{d}, R^{e} and R^{29a} are H; R^{29b} is CF₃; and R² is 2-chloro-5-thiazolyl.

### Table 4

Table 4 is constructed the same as Table 1, except that R^{29b} is CHF₂. For example, the first compound in Table 4 is the compound of Formula T-1 wherein R^{a} is —OCN; R^{b}, R^{c}, R^{d}, R^{e} and R^{29a} are H; R^{29b} is CHF₂; and R² is 2-chloro-5-thiazolyl.

### Table 5

Table 5 is constructed the same as Table 1, except that R^{29a} is F and R^{29b} is H. For example, the first compound in Table 5 is the compound of Formula T-1 wherein R^{a} is —OCN; R^{b}, R^{c}, R^{d}, R^{e} and R^{29b} are H; R^{29a} is F; and R² is 2-chloro-5-thiazolyl.

### Table 6

Table 6 is constructed the same as Table 1, except that R^{29a} is F and R^{29b} is F. For example, the first compound, in Table 6 is the compound of Formula T-1 wherein R^{a} is —OCN; R^{b}, R^{c}, R^{d} and R^{e} are H; R^{29a} and R^{29b} are F; and R² is 2-chloro-5-thiazolyl.

### Table 7

Table 7 is constructed the same as Table 1, except that R^{29a} is F and R^{29b} is CF₃. For example, the first compound in Table 7 is the compound of Formula T-1 wherein R^{a} is —OCN; R^{b}, R^{c}, R^{d} and R^{e} are H; R^{29a} is F; R^{29b} is CF₃; and R² is 2-chloro-5-thiazolyl.

### Table 8

Table 8 is constructed the same as Table 1, except that R² is 6-chloro-3-pyridinyl. For example, the first compound in Table 8 is the compound of Formula T-1 wherein R^{a} is —OCN; R^{b}, R^{c}, R^{d}, R^{e}, R^{29a} and R^{29b} are H; and R² is 6-chloro-3-pyridinyl.

### Table 8a

Table 8a is constructed the same as Table 1, except that R² is 6-bromo-3-pyridinyl. For example, the first compound in Table 8a is the compound of Formula T-1 wherein R^{a} is —OCN; R^{b}, R^{c}, R^{d}, R^{e}; R^{29a} and R^{29b} are H; and R² is 6-bromo-3-pyridinyl.

### Table 8b

Table 8b is constructed the same as Table 1, except that R² is 6-methyl-3-pyridinyl. For example, the first compound in Table 8b is the compound of Formula T-1 wherein R^{a} is —OCN; R^{b}, R^{c}, R^{d}, R^{e}, R^{29a} and R^{29b} are H; and R² is 6-methyl-3-pyridinyl.

### Table 8c

Table 8c is constructed the same as Table 1, except that R² is 6-methyl-3-pyridinyl. For example, the first compound in Table 8c is the compound, of Formula T-1 wherein R^{a} is —OCN; R^{b}, R^{c}, R^{d}, R^{e}, R^{29a} and R^{29b} are H; and R² is 3-pyridinyl.

### Table 9

Table 9 is constructed the same as Table 1, except that R² is 5-thiazolyl. For example, the first compound in Table 9 is the compound of Formula T-1 wherein R^{a} is —OCN; R^{b}, R^{c}, R^{d}, R^{e}, R^{29a} and R^{29b} are H; and R² is 5-thiazolyl.

### Table 10

Table 10 is constructed the same as Table 1, except that R² is 2-methyl-5-thiazolyl. For example, the first compound in Table 10 is the compound of Formula T-1 wherein R^{a} is —OCN; R^{b}, R^{c}, R^{d}, R^{e}, R^{29a} and R^{29b} are H; and R² is 2-methyl-5-thiazolyl.

### Table 11

Table 11 is constructed the same as Table 1, except that R² is 6-fluoro-3-pyridinyl. For example, the first compound in Table 11 is the compound of Formula T-1 wherein R^{a} is —OCN; R^{b}, R^{c}, R^{d}, R^{e}, R^{29a} and R^{29b} are H; and R² is 6-fluoro-3-pyridinyl.

### Table 12

Table 12 is constructed the same as Table 1, except that R² is 2-bromo-5-thiazolyl. For example, the first compound in Table 12 is the compound of Formula T-1 wherein R^{a} is —OCN; R^{b}, R^{c}, R^{d}, R^{e}, R^{29a} and R^{29b} are H; and R² is 2-bromo-5-thiazolyl.

### Table 12a

Table 12a is constructed the same as Table 1, except that R² is 2-fluoro-5-thiazolyl. For example, the first compound in Table 12a is the compound of Formula T-1 wherein R^{a} is —OCN; R^{b}, R^{c}, R^{d}, R^{e}, R^{29a} and R^{29b} are H; and R² is 2-fluoro-5-thiazolyl.

### Table 13

Table 13 is constructed the same as Table 1, except that R² is 4-pyrimidinyl. For example, the first compound in Table 13 is the compound, of Formula T-1 wherein R^{a} is —OCN: R^{b}, R^{c}, R^{d}, R^{e}, R^{29a} and R^{29b} are H; and R² is 4-pyrimidinyl.

### Table 13a

Table 13a is constructed the same as Table 1, except that R² is 4-pyrimidinyl. For example, the first compound in Table 13a is the compound of Formula T-1 wherein R^{a} is —OCN; R^{b}, R^{c}, R^{d}, R^{e}, R^{29a} and R^{29b} are H; and R² is 2-methyl-4-pyrimidinyl.

### Table 14

Table 14 is constructed the same as Table 1, except that R² is N-methyl-4-pyrazolyl. For example, the first compound in Table 14 is the compound of Formula T-1 wherein R^{a} is —OCN; R^{b}, R^{c}, R^{d}, R^{e}, R^{29a} and R^{29b} are H; and R² is N-methyl-4-pyrazolyl.

### Table 15

Table 15 is constructed the same as Table 1, except that R² is CF₃. For example, the first compound in Table 15 is the compound of Formula T-1 wherein R^{a} is —OCN; R^{b}, R^{c}, R^{d}, R^{e}, R^{29a} and R^{29b} are H; and R² is CF₃.

### Table 16

Table 16 is constructed the same as Table 1, except that the structure of Formula T-1 under the Table heading is replaced with the structure of Formula T-2 as follows:

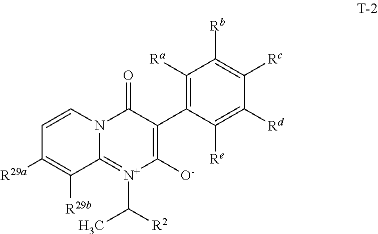

For example, the first compound in Table 16 has the molecular structure shown immediately above wherein R^{a} is —OCN; R^{b}, R^{c}, R^{d}, R^{e}, R^{29a} and R^{29b} are H; and R² is 2-chloro-5-thiazolyl.

TABLE 17

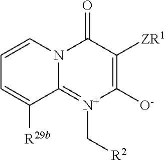

| | | |
|---|---|---|
| ZR¹ | ZR¹ | ZR¹ |

R² is 2-chloro-5-thiazolyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 2-chloro-5-thiazolyl; R^{29b} is F

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 2-chloro-5-thiazolyl; R^{29b} is CF₃

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 2-chloro-5-thiazolyl; R^{29b} is CHF₂

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 2-bromo-5-thiazolyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 5-thiazolyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 2-methyl-5-thiazolyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 6-chloro-3-pyridinyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 6-fluoro-3-pyridinyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 4-pyrimidinyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is N-methyl-4-pyrazolyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is CF₃; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 2-fluoro-5-thiazolyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 6-bromo-3-pyridinyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 6-methyl-3-pyridinyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 3-pyridinyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

R² is 2-methyl-4-pyrimidinyl; R^{29b} is H

| | | |
|---|---|---|
| 6-(2,2-difluorocyclopropyl)-2-pyridinyl | 2-(CH₂OCF₃)-4-pyridinyl | cyano |
| 2-(2,2-difluorocyclopropyl)-4-pyridinyl | 2-(CH₂OCHF₂)-4-pyridinyl | NHPh |
| 2-(cyclopropyl)-4-pyridinyl | C(O)CH₂Ph | |

TABLE 18

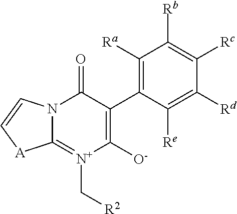

A is O; R^{b}, R^{c}, R^{d} and R^{e} are H; R² is 2-chloro-5-thiazolyl

| R^{a} | R^{a} |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | C(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro-3-pyridinyl) |
| C(═NOEt)Me | |

A is O; R^{a}, R^{c}, R^{d} and R^{e} are H; R² is 2-chloro-5-thiazolyl

| R^{b} | R^{b} |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | C(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro-3-pyridinyl) |
| C(═NOEt)Me | |

A is O; R^{a}, R^{b}, R^{d} and R^{e} are H; R² is 2-chloro-5-thiazolyl

| R^{c} | R^{c} |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | C(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro-3-pyridinyl) |
| C(═NOEt)Me | |

A is S; R^{b}, R^{c}, R^{d} and R^{e} are H; R² is 2-chloro-5-thiazolyl

| R^{a} | R^{a} |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | C(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro-3-pyridinyl) |
| C(═NOEt)Me | |

A is S; R^{a}, R^{c}, R^{d} and R^{e} are H; R² is 2-chloro-5-thiazolyl

| R^{b} | R^{b} |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | C(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro-3-pyridinyl) |
| C(═NOEt)Me | |

A is S; R^{a}, R^{b}, R^{d} and R^{e} are H; R² is 2-chloro-5-thiazolyl

| R^{c} | R^{c} |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | C(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro-3-pyridinyl) |
| C(═NOEt)Me | |

A is NMe; R^{b}, R^{c}, R^{d} and R^{e} are H; R² is 2-chloro-5-thiazolyl

| R^{a} | R^{a} |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | C(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro-3-pyridinyl) |
| C(═NOEt)Me | |

A is NMe; R^{a}, R^{c}, R^{d} and R^{e} are H; R² is 2-chloro-5-thiazolyl

| R^{b} | R^{b} |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | C(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro-3-pyridinyl) |
| C(═NOEt)Me | |

A is NMe; R^{a}, R^{b}, R^{d} and R^{e} are H; R² is 2-chloro-5-thiazolyl

| R^{c} | R^{c} |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | C(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro-3-pyridinyl) |
| C(═NOEt)Me | |

TABLE 19

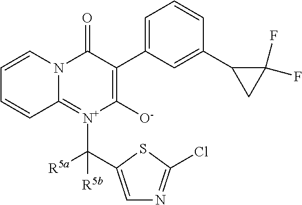

| | | | | | | |
|---|---|---|---|---|---|---|
| R^{5b} | R^{5b} | R^{5b} | R^{5b} | R^{5b} | R^{5b} | R^{5b} |

R^{5a} is H

| | | | | | | |
|---|---|---|---|---|---|---|
| Cl | CHO | Et | OMe | CH═CH₂ | OPh | 3-fluoro-2- |
| | | | | | | pyridinyl |
| Br | C(O)Me | i-Pr | OEt | CL≡CH | NMe₂ | 4-methyl-3- |
| | | | | | | pyridinyl |
| F | CO₂Et | c-Pr | OCF₃ | SMe | NHCHO | 2-cyano-4- |
| | | | | | | pyridinyl |
| Ph | C(O)NMe₂ | CF₃ | Me | cyano | | |

R^{5a} is Me

| | | | | | | |
|---|---|---|---|---|---|---|
| Cl | CHO | Et | OMe | CH═CH₂ | OPh | 3-fluoro-2- |
| | | | | | | pyridinyl |
| Br | C(O)Me | i-Pr | OEt | CL≡CH | NMe₂ | 4-methyl-3- |
| | | | | | | pyridinyl |
| F | CO₂Et | c-Pr | OCF₃ | SMe | NHCHO | 2-cyano-4- |
| | | | | | | pyridinyl |
| Ph | C(O)NMe₂ | CF₃ | Me | cyano | | |

R^{5a} is c-Pr

| | | | | | | |
|---|---|---|---|---|---|---|
| Cl | CHO | Et | OMe | CH═CH₂ | OPh | 3-fluoro-2- |
| | | | | | | pyridinyl |
| Br | C(O)Me | i-Pr | OEt | CL≡CH | NMe₂ | 4-methyl-3- |
| | | | | | | pyridinyl |
| F | CO₂Et | c-Pr | OCF₃ | SMe | NHCHO | 2-cyano-4- |
| | | | | | | pyridinyl |
| Ph | C(O)NMe₂ | CF₃ | Me | cyano | | |

TABLE 20

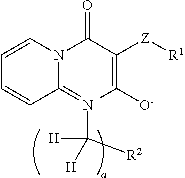

| | | |
|---|---|---|
| ZR¹ | ZR¹ | ZR¹ |

a is 2; R² is 2-chloro-5-thiazolyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 2; R² is 2-methyl-5-thiazolyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 2; R² is 5-thiazolyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 2; R² is CF₃

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 2; R² is 6-chloro-3-pyridinyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 2; R² is 6-fluoro-3-pyridinyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 2; R² is 1-methyl-4-pyrazolyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 3; R² is 2-chloro-5-thiazolyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 3; R² is 2-methyl-5-thiazolyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 3; R² is 5-thiazolyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 3; R² is CF₃

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 3; R² is 6-chloro-3-pyridinyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 3; R² is 6-fluoro-3-pyridinyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

a is 3; R² is 1-methyl-4-pyrazolyl

| | | |
|---|---|---|
| phenyl | 2-fluorophenyl | 3-(trifluoromethyl)phenyl |
| CO₂Et | 3-methoxyphenyl | 3-(trifluoromethoxy)phenyl |
| C(O)Ph | 3-(CO₂Et)phenyl | 3-chloro-5-(trifluoromethyl)phenyl |
| C(O)CF₃ | 3-(C(O)NMe₂)phenyl | 3-(2-chloro-4-cyanophenyl)phenyl |
| | 3,5-dichlorophenyl | 3-(2-chloro-4- |
| | | (trifluoromethyl)phenyl)phenyl |
| | | 3-(2-chloro-4-(trifluoromethyl)phenyl)- |
| | | 5-methylphenyl |
| | | 3-(2-chloro-4-(trifluoromethyl)phenyl)- |
| | | 5-methoxyphenyl |

TABLE 21

| 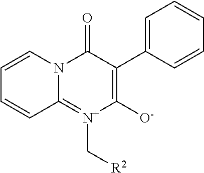 | | | |
|---|---|---|---|
| R² | R² | R² | R² |
| cyano | C(O)Me | OPh | 3-tetrahydrofuranyl |
| CHO | CH₂OCF₃ | CH₂NMe₂ | 1,4-dioxanyl |
| Ph | C(O)NMe₂ | CH₂NHCHO | |

TABLE 22

| 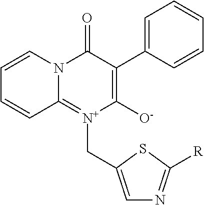 | |
|---|---|
| R | R |
| CHO | OPh |
| NHCHO | NMe₂ |

TABLE 23

| 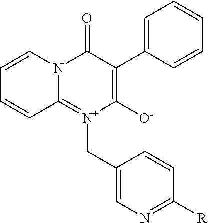 | |
|---|---|
| R | R |
| CHO | OPh |
| NHCHO | NMe₂ |

TABLE 24

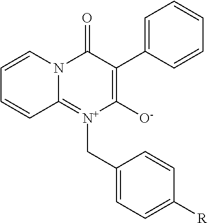

| R | R | R | R | R | R | R |
|---|---|---|---|---|---|---|
| cyano | C(O)Me | Et | OMe | CH═CH₂ | OPh | 3-fluoro-2- |
| | | | | | | pyridinyl |
| CHO | CO₂Et | i-Pr | OEt | C≡CH | NMe₂ | 4-methyl-3- |
| | | | | | | pyridinyl |
| Ph | C(O)NMe₂ | c-Pr | OCF₃ | SMe | NHCHO | 2-cyano-4- |
| | | | | | | pyridinyl |
| CF₃ | Me | Cl | Br | | | |

TABLE 25

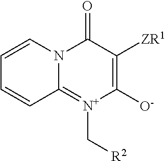

| | | | |
|---|---|---|---|
| ZR¹ | ZR¹ | ZR¹ | ZR¹ |

R² is 2-chloro-5-thiazolyl

| | | | |
|---|---|---|---|
| —OCN | CH₂OCH₃ | S(O)—c-Pr | OCOCF₃ |
| CN | CH₂OCF₃ | S(O)₂—c-Pr | N(Me)COCF₃ |
| CHO | CH₂OCHF₂ | CH₂CN | OCH₂CH═CH₂ |
| NMe₂ | SCF₃ | CH═CHCN | OCH₂C≡CH |
| NHCHO | S—c-Pr | C≡CCH₂CN | O—c-Pr |

R² is 6-chloro-3-pyridinyl

| | | | |
|---|---|---|---|
| —OCN | CH₂OCH₃ | S(O)—c-Pr | OCOCF₃ |
| CN | CH₂OCF₃ | S(O)₂—c-Pr | N(Me)COCF₃ |
| CHO | CH₂OCHF₂ | CH₂CN | OCH₂CH═CH₂ |
| NMe₂ | SCF₃ | CH═CHCN | OCH₂C≡CH |
| NHCHO | S—c-Pr | C≡CCH₂CN | O—c-Pr |

R² is 6-fluoro-3-pyridinyl

| | | | |
|---|---|---|---|
| —OCN | CH₂OCH₃ | S(O)—c-Pr | OCOCF₃ |
| CN | CH₂OCF₃ | S(O)₂—c-Pr | N(Me)COCF₃ |
| CHO | CH₂OCHF₂ | CH₂CN | OCH₂CH═CH₂ |
| NMe₂ | SCF₃ | CH═CHCN | OCH₂C≡CH |
| NHCHO | S—c-Pr | C≡CCH₂CN | O—c-Pr |

TABLE 26

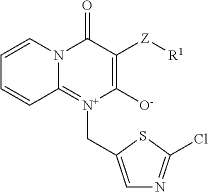

| | | | | | | |
|---|---|---|---|---|---|---|
| R¹ | R¹ | R¹ | R¹ | R¹ | R¹ | R¹ |

Z is O

| | | | | | | |
|---|---|---|---|---|---|---|
| Ph | 2-F—Ph | 3-F—Ph | 4-F—Ph | 2-MeO—Ph | 3-MeO—Ph | 4-MeO—Ph |
| 2-Me—Ph | 3-Me—Ph | 4-Me—Ph | 2-CF₃—Ph | 3-CF₃—Ph | 4-CF₃—Ph | 2-CF₃O—Ph |
| 3-CF₃O—Ph | 4-CF₃O—Ph | Me | Et | i-Pr | c-Pr | CF₃ |
| 2-c-Pr—c-Pr | C(O)Me | CO₂Et | C(O)NMe₂ | | | |

Z is NMe

| | | | | | | |
|---|---|---|---|---|---|---|
| Ph | 2-F—Ph | 3-F—Ph | 4-F—Ph | 2-MeO—Ph | 3-MeO—Ph | 4-MeO—Ph |
| 2-Me—Ph | 3-Me—Ph | 4-Me—Ph | 2-CF₃—Ph | 3-CF₃—Ph | 4-CF₃—Ph | 2-CF₃O—Ph |
| 3-CF₃O—Ph | 4-CF₃O—Ph | Me | Et | i-Pr | c-Pr | CF₃ |
| 2-c-Pr—c-Pr | C(O)Me | CO₂Et | C(O)NMe₂ | | | |

TABLE 27

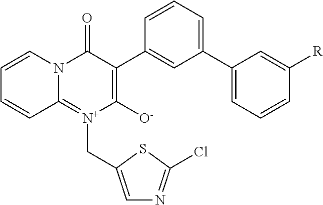

| R | R |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | CO(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro -3-pyridinyl) |
| C(═NOEt)Me | |

TABLE 28

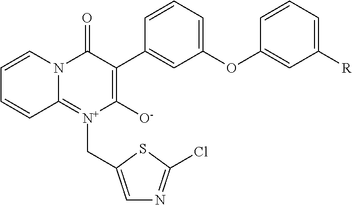

| R | R |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | CO(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro -3-pyridinyl) |
| C(═NOEt)Me | |

TABLE 29

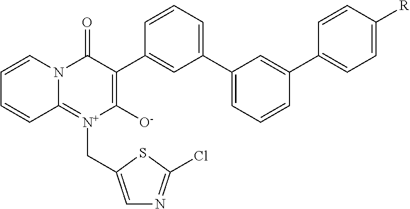

| R | R |
|---|---|
| —OC≡N | cyclopropylthio |
| cyclopropyl | cyclopropylsulfinyl |
| 2-methylcyclopropyl | cyclopropylsulfonyl |
| 2,2-difluorocyclopropyl | CH₂CN |
| CH₂(cyclopropyl) | CH₂CO₂Et |
| 2,2-dichlorocyclopropyl | CH═CHCN |
| CH₂OCF₃ | C≡CCH₂CN |
| CH₂OCHF₂ | CO₂CH₂CF₃ |
| OC(O)CF₃ | CH₂(2,2-dichlorocyclopropyl) |
| N(Me)C(O)CF₃ | CO(O)N(Me)CH₂CF₃ |
| OCH₂CH═CH₂ | 3-bromo-4,5-dihydro-isoxazol-5-yl |
| OCH₂C≡CH | 3-methyl-4,5-dihydro-isoxazol-5-yl |
| cyclopropoxy | OCH₂(2-pyridinyl) |
| OCH₂CN | OCH₂(6-chloro-2-pyridinyl) |
| OCH₂CF₃ | OCH₂(3-pyridinyl) |
| C(═NOMe)Me | OCH₂(6-chloro -3-pyridinyl) |
| C(═NOEt)Me | |

A compound of this invention will generally be used as an invertebrate pest control active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed, into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied, onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Borland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-bexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g, oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids can be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified, as nonionic, anionic or cationic. Nonionic surfactants useful for the present compositions include, but are not limited to: alcohol alkoxylates such as alcohol alkoxylates based on natural and synthetic alcohols (which are branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates: protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention can also contain formulation auxiliaries and additives, known to those skilled in the art as formulation aids (some of which can be considered to also function as solid diluents, liquid diluents or surfactants). Such formulation auxiliaries and additives can control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared, by blending and usually grinding (such as with a hammer mill or fluid-energy mill). Granules and pellets can be prepared, by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col, 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al, Weed Control Handbook, 8th Ed., Blackwell Scientific Publications, Oxford, 1989; and Developments in formulation technology, PJB Publications, Richmond, UK, 2000.

In the following Examples, all formulations are prepared in conventional ways. Compound numbers refer to compounds in Index Table A. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

### High Strength Concentrate

| | |
|---|---|
| compound 88 | 98.5% |
| silica aerogel | 0.5% |
| synthetic amorphous fine silica | 1.0% |

### Example B

### Wettable Powder

| | |
|---|---|
| compound 92 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

### Granule

| | |
|---|---|
| compound 93 | 10.0% |
| attapulgite granules (low volatile matter, 0.71/0.30 mm; | 90.0% |
| U.S.S. No. 25-50 sieves) | |

### Example D

### Extruded Pellet

| | |
|---|---|
| compound 102 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

### Emulsifiable Concentrate

| | |
|---|---|
| compound 105 | 10.0% |
| polyoxyethylene sorbitol hexoleate | 20.0% |
| C₆-C₁₀ fatty acid methyl ester | 70.0% |

### Example F

### Microemulsion

| | |
|---|---|
| compound 107 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

### Seed Treatment

| | |
|---|---|
| compound 113 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

### Fertilizer Stick

| | |
|---|---|
| compound 92 | 2.50% |
| pyrrolidone-styrene copolymer | 4.80% |
| tristyrylphenyl 16-ethoxylate | 2.30% |
| talc | 0.80% |
| corn starch | 5.00% |
| slow-release fertilizer | 36.00% |
| kaolin | 38.00% |
| water | 10.60% |

### Example I

### Suspension Concentrate

| | |
|---|---|
| compound 102 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example J

### Emulsion in Water

| | |
|---|---|
| compound 105 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example K

### Oil Dispersion

| | |
|---|---|
| compound 107 | 25% |
| polyoxyethylene sorbitol hexaoleate | 15% |
| organically modified bentonite clay | 2.5% |
| fatty acid methyl ester | 57.5% |

### Example L

### Suspoemulsion

| | |
|---|---|
| compound 113 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Compounds of this invention exhibit activity against a wide spectrum of invertebrate pests. These pests include invertebrates inhabiting a variety of environments such as, for example, plant foliage, roots, soil, harvested crops or other foodstuffs, building structures or animal integuments. These pests include, for example, invertebrates feeding on foliage (including leaves, stems, flowers and fruits), seeds, wood, textile fibers or animal blood or tissues, and thereby causing injury or damage to, for example, growing or stored agronomic crops, forests, greenhouse crops, ornamentals, nursery crops, stored foodstuffs or fiber products, or houses or other structures or their contents, or being harmful to animal health or public health. Those skilled in the art will appreciate that not all compounds are equally effective against all growth stages of all pests.

These present compounds and compositions are thus useful agronomically for protecting field crops from phytophagous invertebrate pests, and also nonagronomically for protecting other horticultural crops and plants from phytophagous invertebrate pests. This utility includes protecting crops and other plants (i.e. both agronomic and nonagronomic) that contain genetic material introduced by genetic engineering (i.e. transgenic) or modified by mutagenesis to provide advantageous traits. Examples of such traits include tolerance to herbicides, resistance to phytophagous pests (e.g., insects, mites, aphids, spiders, nematodes, snails, plant-pathogenic fungi, bacteria and viruses), improved plant growth, increased tolerance of adverse growing conditions such as high or low temperatures, low or high soil moisture, and high salinity, increased flowering or fruiting, greater harvest yields, more rapid, maturation, higher quality and/or nutritional value of the harvested product, or improved, storage or process properties of the harvested products. Transgenic plants can be modified to express multiple traits. Examples of plants containing traits provided by genetic engineering or mutagenesis include varieties of corn, cotton, soybean and potato expressing an insecticidal Bacillus thuringiensis toxin such as YIELD CARD®, KNOCKOUT®, STARLINK®, BOLLGARD®, NuCOTN® and NEWLEAF®, and herbicide-tolerant varieties of corn, cotton, soybean and rapeseed such as ROUNDUP READY®, LIBERTY LINK®, IMF®, STS® and CLEARFIELD®, as well as crops expressing N-acetyltransferase (GAT) to provide resistance to glyphosate herbicide, or crops containing the HRA gene providing resistance to herbicides inhibiting acetolactate synthase (ALS). The present compounds and compositions may interact synergistically with traits introduced by genetic engineering or modified by mutagenesis, thus enhancing phenotypic expression or effectiveness of the traits or increasing the invertebrate pest control effectiveness of the present compounds and compositions. In particular, the present compounds and compositions may interact synergistically with the phenotypic expression of proteins or other natural products toxic to invertebrate pests to provide greater-than-additive control of these pests.

Compositions of this invention can also optionally comprise plant nutrients, e.g., a fertilizer composition comprising at least one plant nutrient selected from nitrogen, phosphorus, potassium, sulfur, calcium, magnesium, iron, copper, boron, manganese, zinc, and molybdenum. Of note are compositions comprising at least one fertilizer composition comprising at least one plant nutrient selected from nitrogen, phosphorus, potassium, sulfur, calcium and magnesium. Compositions of the present invention which further comprise at least one plant nutrient can be in the form of liquids or solids. Of note are solid formulations in the form of granules, small sticks or tablets. Solid formulations comprising a fertilizer composition can be prepared, by mixing the compound or composition of the present invention with the fertilizer composition together with formulating ingredients and then preparing the formulation by methods such as granulation or extrusion. Alternatively solid formulations can be prepared, by spraying a solution or suspension of a compound, or composition of the present invention in a volatile solvent onto a previous prepared fertilizer composition in the form of dimensionally stable mixtures, e.g., granules, small sticks or tablets, and then evaporating the solvent.

Examples of agronomic or nonagronomic invertebrate pests include eggs, larvae and adults of the order Lepidoptera, such as armyworms, cutworms, loopers, and heliothines in the family Noctuidae (e.g., pink stem borer (Sesamia inferens Walker), corn stalk borer (Sesamia nonagrioides Lefebvre), southern armyworm (Spodoptera eridania Cramer), fall armyworm (Spodoptera fugiperda J. E. Smith), beet armyworm (Spodoptera exigua Hübner), cotton leafworm (Spodoptera littoralis Boisduval), yellowstriped armyworm (Spodoptera ornithogalli Guenée), black cutworm (Agrotis ipsilon Hufnagel), velvetbean caterpillar (Anticarsia gemmatalis Hübner), green fruitworm (Lithophane antennata Walker), cabbage armyworm (Barathra hrassicae Linnaeus), soybean looper (Pseudoplusia includens Walker), cabbage looper (Trichoplusia ni Hübner), tobacco budworm (Heliothis virescens Fabricius)); borers, casebearers, webworms, coneworms, cabbageworms and skeletonizers from the family Pyralidae (e.g., European corn borer (Ostrinia nubilalis Hübner), navel orangeworm (Amyelois transitella Walker), corn root webworm (Crambus caliginosellus Clemens), sod webworms (Pyralidae: Crambinae) such as sod worm (Hetpetogramma licarsisalis Walker), sugarcane stem borer (Chilo infuscatellus Snellen), tomato small borer (Neoleucinodes elegantalis Guenée), green leafroller (Cnaphalocerus medinalis), grape leaffolder (Desmia funeralis Hübner), melon worm (Diaphania nitidalis Stoll), cabbage center grab (Helluala hydralis Guenée), yellow stem borer (Scirpophaga incertulas Walker), early shoot borer (Scirpophaga infuscatellus Snellen), white stem borer (Scirpophaga innotata Walker), top shoot borer (Scirpophaga nivella Fabricius), dark-headed rice borer (Chilo polychrysus Meyrick), cabbage cluster caterpillar (Crocidolomia binotalis English)); leafrollers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck), citrus false codling moth (Cryptophlebia leucotreta Meyrick), citrus borer (Ecdytolopha aurantiana Lima), redbanded leafroller (Argyrotaenia velutinana Walker), obliquebanded leafroller (Choristoneura rosaceana Harris), light brown apple moth (Epiphyas postvittana Walker), European grape berry moth (Eupoecilia ambiguella Hübner), apple bud moth (Pandemis pyrusana Kearfott), omnivorous leafroller (Platynota stultana Waisingham), barred fruit-tree tortrix (Pandemis cerasana Hübner), apple brown tortrix (Pandemis heparana Denis & Schiffermüller)); and many other economically important lepidoptera (e.g., diamondback moth (Plutella xylosiella Linnaeus), pink boilworm (Pectinophora gossypiella Saunders), gypsy moth (Lymaniria dispar Linnaeus), peach fruit borer (Carposina niponensis Waisingham), peach twig borer (Anarsia lineatella Zeller), potato luberworm (Phihorimaea operculella Zeller), spotted teniform leafminer (Lithocolletis blancardella Fabricius), Asiatic apple leafminer (Lithocolletis ringoniella Matsumura), rice leaffolder (Lerodea eufala Edwards), apple leafminer (Leucoptera scitella Zeller)); eggs, nymphs and adults of the order Blattodea including cockroaches from the families Biattellidae and Blattidae (e.g., oriental cockroach (Blatta orientalis Linnaeus), Asian cockroach (Blatella asahinai Mizukubo), German cockroach (Blattella germanica Linnaeus), brownbanded cockroach (Supella longipalpa Fabricius), American cockroach (Periplaneta americana Linnaeus), brown cockroach (Periplaneta brumnea Burmeister), Madeira cockroach (Leucophaea maderae Fabricius)), smoky brown cockroach (Periplaneta fuliginosa Service), Australian Cockroach (Periplaneta australasiae Fabr.), lobster cockroach (Nauphoeta cinema Olivier) and smooth cockroach (Symploce pallens Stephens)); eggs, foliar feeding, fruit feeding, root feeding, seed, feeding and vesicular tissue feeding larvae and adults of the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)), annual bluegrass weevil (Listronotus maculicollis Dietz), bluegrass billbug (Sphenophorus parvulus Gyllenhal), hunting billbug (Sphenophorus venatus vestitus), Denver billbug (Sphenophorus cicatristriatus Fahraeus)); flea beetles, cucumber beetles, rootworms, leaf beetles, potato beetles, and leafminers in the family Chrysomeiidae (e.g., Colorado potato beetle (Leptinotarsa decemlineata Say), western corn rootworm (Diabrotica virgifera virgifera LeConte)); chafers and other beetles from the family Scarabaeidae (e.g., Japanese beetle (Popillia japonica Newman), oriental beetle (Anomala orientalis Waterhouse, Exomala orientalis (Waterhouse) Baraud), northern masked chafer (Cyclocephala borealis Arrow), southern masked chafer (Cyclocephala immaculata Olivier or C. lurida Bland), dung beetle and white grub (Aphodius spp.), black turfgrass ataenius (Ataenius spretulus Haldeman), green June beetle (Cotinis nitida Linnaeus), Asiatic garden beetle (Maladera casianea Arrow), May/June beetles (Phyllophaga spp.) and European chafer (Rhizotrogus majalis Razoumowsky)); carpet beetles from the family Dermestidae; wireworms from the family Elateridae; bark beetles from the family Scolytidae and flour beetles from the family Tenebrionidae. In addition, agronomic and nonagronomic pests include: eggs, adults and larvae of the order Dermaptera including earwigs from the family Forficulidae (e.g., European earwig (Forjicula auricularia Linnaeus), black earwig (Chelisoches morio Fabricius)); eggs, immatures, adults and nymphs of the orders Hemiptera and Homoptera such as, plant bugs from the family Miridae, cicadas from the family Cicadidae, leafhoppers (e.g. Empoasca spp.) from the family Cicadellidae, bed bugs (e.g., Cimex leclularius Linnaeus) from the family Cimicidae, planthoppers from the families Fulgoroidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, chinch bugs (e.g. hairy chinch bug (Blissus leucopterus hirtus Montandon) and southern chinch bug (Blissus insularis Barber)) and other seed bugs from the family Lygaeidae, spittlebugs from the family Cercopidae squash bugs from the family Coreidae, and red bugs and cotton stainers from the family Pyrrbocoridae. Also included are eggs, larvae, nymphs and adults of the order Acari (mites) such as spider mites and red mites in the family Tetranycbidae (e.g., European red mite (Panonychus ulmi Koch), two spotted spider mite (Tetranychus urticae Koch), McDaniel mite (Tetranychus mcdanieli McGregor)); flat mites in the family Tenuipalpidae (e.g., citrus flat mite (Brevipalpus lewisi McGregor)); rust and bud mites in the family Eriophyidae and other foliar feeding mites and mites important in human and animal health, i.e. dust mites in the family Epidermoptidae, follicle mites in the family Demodicidae, grain mites in the family Glycyphagidae; ticks in the family Ixodidae, commonly known as hard ticks (e.g., deer tick (Ixodes scapularis Say), Australian paralysis tick (Ixodes holocyclus Neumann), American dog tick (Dermacentor variabilis Say), lone star tick (Amblyomma americanum Linnaeus)) and ticks in the family Argasidae, commonly known as soft ticks (e.g., relapsing fever tick (Ornithodoros turicata), common fowl tick (Argas radiatus)); scab and itch mites in the families Psoroptidae, Pyemotidae, and Sarcoptidae; eggs, adults and immatures of the order Orthoptera including grasshoppers, locusts and crickets (e.g., migratory grasshoppers (e.g., Melanoplus sanguinipes Fabricius, M. dijferentialis Thomas), American grasshoppers (e.g., Schisiocerca americana Drury), desert locust (Schistocerca gregaria Forskal), migratory locust (Locusta migratoria Linnaeus), bush locust (Zonocerus spp.), house cricket (Acheta domesticus Linnaeus), mole crickets (e.g., tawny mole cricket (Scapteriscus vicinus Scudder) and southern mole cricket (Scapteriscus borellii Giglio-Tos)); eggs, adults and immatures of the order Diptera including leafminers (e.g., Liriomyza spp. such as serpentine vegetable leafminer (Liriomyza sativae Blanchard)), midges, fruit flies (Tephritidae), frit flies (e.g., Oscinella frit Linnaeus), soil maggots, house flies (e.g., Musca domestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chrysoniya spp., Phormia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; eggs, adults and immatures of the order Thysanoptera including onion thrips (Thrips tabaci Lindeman), flower thrips (Frankliniella spp.), and other foliar feeding thrips; insect pests of the order Hymenoptera including ants of the Family Formicidae including the Florida carpenter ant (Camponotus floridanus Buckley), red carpenter ant (Camponotus ferrugineus Fabricius), black carpenter ant (Camponotus pennsyivanicus De Geer), white-footed ant (Technomyrmex albipes fr. Smith), big headed ants (Pheidole sp.), ghost ant (Tapinoma melanocephalum Fabricius); Pharaoh ant (Monomorium pharaonis Linnaeus), little fire ant (Wasmannia auropunctata Roger), fire ant (Solenopsis gerninata Fabricius), red imported fire ant (Solenopsis invicia Buren), Argentine ant (Iridomyrmex humilis Mayr), crazy ant (Paratrechina longicornis Latreille), pavement ant (Tetramorium caespitum Linnaeus), cornfield ant (Lasius alienus Förster) and odorous house ant (Tapinoma sessile Say). Other Hymenoptera including bees (including carpenter bees), hornets, yellow jackets, wasps, and sawflies (Neodiprion spp.; Cephas spp.); insect pests of the order Isoptera including termites in the Termitidae (e.g., Macrotermes sp., Odontotermes obesus Rambur), Kalotermitidae (e.g., Cryptotermes sp.), and Rhinotermitidae (e.g., Reticulitermes sp., Coptotermes sp., Heterotermes tenuis Hagen) families, the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder), powder post termite (Cryptotermes brevis Walker), drywood termite (Incisitermes snyderi Light), southeastern subterranean termite (Reticulitermes virginicus Banks), western drywood termite (Incisitermes minor Hagen), arboreal termites such as Nasutiiermes sp. and other termites of economic importance; insect pests of the order Thysanura such as silverfish (Lepisma saccharina Linnaeus) and firebrat (Thermobia domestica Packard); insect pests of the order Mallophaga and including the head louse (Pediculus humanus capitis De Geer), body louse (Pediculus humanus Linnaeus), chicken body louse (Menacanthus stramineus Nitszeh), dog biting louse (Trichodectes canis De Geer), fluff louse (Goniocotes gallinae De Geer), sheep body louse (Bovicola ovis Schrank), short-nosed cattle louse (Haematopinus eurysternus Nitzsch), long-nosed, cattle louse (Linognathus vituli Linnaeus) and other sucking and chewing parasitic lice that attack man and animals; insect pests of the order Siphonoptera including the oriental rat flea (Xenopsylla cheopis Rothschild), cat flea (Ctenocephalides felis Bouche), dog flea (Ctenocephalides canis Curtis), hen flea (Ceratophyllus gallinae Schrank), sticktight flea (Echidnophaga gallinacea Westwood), human flea (Pulex irritans Linnaeus) and other fleas afflicting mammals and birds. Additional arthropod pests covered, include: spiders in the order Araneae such as the brown recluse spider (Loxosceles reclusa Gertsch & Mulaik) and the black widow spider (Latrodectus mactans Fabricius), and centipedes in the order Scutigeromorpha such as the house centipede (Scutigera coleoptrata Linnaeus). Compounds of the present invention also have activity on members of the Classes Nematoda, Cestoda, Trematoda, and Acanthocephala including economically important members of the orders Strongylida, Ascaridida, Oxyurida, Rhabditida, Spirurida, and Enoplida such as but not limited to economically important agricultural pests (i.e. root knot nematodes in the genus Meloidogyne, lesion nematodes in the genus Pratylenchus, stubby root nematodes in the genus Trichodorus, etc.) and animal and human health pests (i.e. all economically important flukes, tapeworms, and roundworms, such as Strongylus vulgaris in horses, Toxocara canis in dogs, Haemonchus contortus in sheep, Dirofilaria immitis Leidy in dogs, Anoplocephala perfoliate in horses, Fasciola hepatica Linnaeus in ruminants, etc.).

Compounds of the invention show particularly high activity against pests in the order Lepidoptera (e.g., Alabama argillacea Hübner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenée (rice leaf roller), Crambus caliginosellus Clemens (corn root webworm), Crambos teierrellus Zincken (bluegrass webworm), Cydia pomonella Linnaeus (codling moth), Earias insulana Boisduval (spiny bollworm), Earias vittella Fabricius (spotted, bollworm), Helicoverpa armigera Hübner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermuller (grape berry moth), Pectinophora gossypiella Saunders (pink bollworm), Phyllocnistis citrella Stainton (citrus leafrniner), Pieris brassicae Linnaeus (large white butterfly), Pieris rapae Linnaeus (small white butterfly), Plutetta xylostella Linnaeus (diamondback moth), Spodoptera exigua Hübner (beet armyworm), Spodoptera litura Fabricius (tobacco cutworm, cluster caterpillar), Spodoptera frugiperda J. E. Smith (fall armyworm), Trichoplusia ni Hübner (cabbage looper) and Tula absoluta Meyrick (tomato leafminer)).

Compounds of the invention also have significant activity on members from the order Homoptera including: Acyrthosiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffrey (mealy plum aphid), Lipaphis erysimi Kaltenbach (turnip aphid), Metopolophium dirrhodum Walker (cereal aphid), Macrosiphum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird, cherry-oat aphid), Schizaphis graminum Rondani (greenbug), Sitobion avenae Fabricius (English grain aphid), Therioaphis maculate Buckton (spotted alfalfa aphid), Toxoptera aurantii Boyer de Fonscolombe (black citrus aphid), and Toxoptera citricida Kirkaldy (brown citrus aphid); Adelges spp. (adelgids); Phylloxera devastatrix Pergande (pecan phylloxera); Bemisia tabaci Gennadius (tobacco whitefiy, sweetpotato whitefiy), Bemisia argentifolii Bellows & Perring (silverleaf whitefiy), Dialeurodes citri Ashmead (citrus whitefly) and Trialeurodes vaporariorum Weslwood (greenhouse whitefly); Empoasca fabae Harris (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stål (rice leafhopper), Nilaparvata lugens Stål (brown planthopper), Peregrinus maidis Ashmead (corn planthopper), Sogatella furcifera Horvath (white-backed planthopper), Sogalodes orizicola Muir (rice delphacid), Typhlocyba pomaria McAfee white apple leafhopper, Erythroneoura spp. (grape leafhoppers); Magicidada septendecim Linnaeus (periodical cicada); Icerya purchasi Maskell (cottony cushion scale), Quadraspidiolus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla).

Compounds of this invention may also have activity on members from the order Hemiptera including: Acrostemum hilare Say (green stink bug), Anasa iristis De Geer (squash bug), Blissus leucopterus leucopterus Say (chinch bug), Cimex lectidarius Linnaeus (bed bug) Corythuca gossypii Fabricius (cotton lace bug), Cyrtopeltis modesta Distant (tomato bug), Dysdercus suturellus Herrich-Schäffer (cotton stainer), Euchistus servus Say (brown stink bug), Euchistus variolarius Palisot de Beauvois (one-spotted stink bug), Graptosthetus spp. (complex of seed bugs), Leptoglossus corculus Say (leaf-footed, pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper). Other insect orders controlled by compounds of the invention include Thysanoptera (e.g., Frankliniella occidentalis Pergande (western flower thrips), Scirthothrips citri Moulton (citrus thrips), Sericothrips variabilis Beach (soybean thrips), and Thrips tabaci Lindeinan (onion thrips); and the order Coleoptera (e.g., Leptinotarsa decemlineata Say (Colorado potato beetle), Epilachna varivestis Mulsant (Mexican bean beetle) and wireworms of the genera Agriotes, Athous or Limonius).

Note that some contemporary classification systems place Homoptera as a suborder within the order Hemiptera.

Of note is use of compounds of this invention for controlling potato leafhopper (Empoasca fabae). Of note is use of compounds of this invention for controlling corn planthopper (Peregrinus maidis). Of note is use of compounds of this invention for controlling cotton melon aphid (Aphis gossypii). Of note is use of compounds of this invention for controlling green peach aphid (Myzus persicae). Of note is use of compounds of this invention for controlling diamondback moth (Plutella xylostella). Of note is use of compounds of this invention for controlling fell armyworm (Spodoptera frugiperda).

Of note is use of compounds of this invention for controlling southern green stink bug (Nezara viridula), western tarnished plant bug (Lygus hesperus), rice water weevil (Lissorhoptrus oryzophilus), rice brown planthopper (Nilaparvata lugens), rice green leafhopper (Nephotettix virescens) and striped rice borer (Chilo suppressalis).

Compounds of this invention can also be mixed with one or more other biologically active compounds or agents including insecticides, fungicides, nematocides, bactericides, acaricides, herbicides, herbicide safeners, growth regulators such as insect molting inhibitors and roofing stimulants, chemosterilants, semiochemicals, repellents, attractants, pheromones, feeding stimulants, other biologically active compounds or entomopathogenic bacteria, virus or fungi to form, a multi-component pesticide giving an even broader spectrum, of agronomic and nonagronomic utility. Thus the present invention also pertains to a composition comprising a biologically effective amount of a compound of Formula 1, an N-oxide or salt thereof, at least one additional component selected from the group consisting of surfactants, solid diluents and liquid diluents, and at least one additional biologically active compound or agent. For mixtures of the present invention, the other biologically active compounds or agents can be formulated, together with the present compounds, including the compounds of Formula 1, to form a premix, or the other biologically active compounds or agents can be formulated separately from the present compounds, including the compounds of Formula 1, and the two formulations combined together before application (e.g., in a spray tank) or, alternatively, applied in succession.

Examples of such biologically active compounds or agents with which compounds of this invention can be formulated are insecticides such as abamectin, acephate, acequinocyl, acetamiprid, acrinathrin, amidoflumet, amitraz, avermectin, azadirachtin, azinphos-methyl, bensultap, bifenthrin, bifenazate, bistrifluoron, borate, buprofezin, cadusafos, carbaryl, carbofuran, cartap, carzol, chlorantraniliprole, chlorfenapyr, chlorfiuazuron, chlorpyrifos, chlorpyrifos-methyl, chromafenozide, clofentezin, clothianidin, cyantraniliprole, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenothiocarb, fenoxycarb, fenpropathrin, fenvalerate, fipronil, flonicamid, flubendiamide, flucythrinate, flufenerim, flufenoxuron, fluvalinate, tau-fluvalinate, fonophos, formetanate, fosthiazate, halofenozide, hexaflumuron, hexythiazox, hydramethylnon, imidacloprid, indoxacarb, insecticidal soaps, isofenphos, lufenuron, malathion, meperfluthrin, metaflumizone, metaldehyde, methamidophos, methidathion, methiodicarb, methomyl, methoprene, methoxychlor, metofluthrin, monocrotophos, methoxyfenozide, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, spinetoram, spinosad, spirodiclofen, spiromesifen, spirotetramat, sulprofos, sulfoxaflor, iebufenozide, tebufenpyrad, teflubenzuron, tefluthrin, terbufos, tetrachlorvinphos, tetramethrin, tetramethylfluthrin, thiacloprid, thiamethoxam, thiodicarb, thiosultap-sodium, tolfenpyrad, tralomethrin, triazamate, trichlorfon, triflumuron, Bacillus thuringiensis delta-endotoxins, entomopathogenic bacteria, entomopathogenic viruses and entomopathogenic fungi.

Of note are insecticides such as abamectin, acetamiprid, acrinathrin, amitraz, avermectin, azadirachtin, bensultap, bifenthrin, buprofezin, cadusafos, carbaryl, cartap, chlorantraniliprole, chlorfenapyr, chlorpyrifos, clothianidin, cyantraniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, flufenoxuron, fluvalinate, formetanate, fosthiazate, hexaflumuron, hydramethylnon, imidacloprid, indoxacarb, lufenuron, metaflumizone, methiodicarb, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, spirotetramat, tebufenozide, tetramethrin, thiacloprid, thiamethoxam, thiodicarb, thiosultap-sodium, tralomethrin, triazamate, triflumuron, Bacillus thuringiensis delta-endotoxins, all strains of Bacillus thuringiensis and all strains of Nucleo polyhydrosis viruses.

One embodiment of biological agents for mixing with compounds of this invention include entomopathogenic bacteria such as Bacillus thuringiensis, and the encapsulated delta-endotoxins of Bacillus thuringiensis such as MVP® and MVPII® bioinsecticides prepared by the CellCap® process (CellCap®, MVP® and MVPII® are trademarks of Mycogen Corporation, Indianapolis, Ind., USA); entomopathogenic fungi such as green muscardine fungus; and entomopathogenic (both naturally occurring and genetically modified) viruses including baculovirus, nucleopolyhedro virus (NPV) such as Helicoverpa zea nucleopolyhedrovirus (HzNPV), Anagrapha falcifera nucieopoiyhedrovirus (AfNPV); and granulosis virus (GV) such as Cydia pomonella granulosis virus (CpGV).

Of particular note is such a combination where the other invertebrate pest control active ingredient belongs to a different chemical class or has a different site of action than the compound of Formula 1. In certain instances, a combination with at least one other invertebrate pest control active ingredient having a similar spectrum of control but a different site of action will be particularly advantageous for resistance management. Thus, a composition of the present invention can further comprise a biologically effective amount of at least one additional invertebrate pest control active ingredient having a similar spectrum of control but belonging to a different chemical class or having a different site of action. These additional biologically active compounds or agents include, but are not limited to, sodium channel modulators such as bifenthrin, cypermethrin, cyhalothrin, lambda-cyhalothrin, cyfluthrin, beta-cyfluthrin, deltamethrin, dimefluthrin, esfenvalerate, fenvalerate, indoxacarb, metofluthrin, profluthrin, pyrethrin and tralomethrin; cholinesterase inhibitors such as chlorpyrifos, methomyl, oxamyl, thiodicarb and triazamate; neonicotinoids such as acetamiprid, clothianidin, dinotefuran, imidacloprid, nitenpyram, nithiazine, thiacloprid and thiamethoxam; insecticidal macrocyclic lactones such as spinetoram, spinosad, abamectin, avermectin and emamectin; GABA (γ-aminobutyric acid)-gated chloride channel antagonists such as avermectin or blockers such as ethiprole and fipronil; chitin synthesis inhibitors such as buprofezin, cyromazine, flufenoxuron, hexaflumuron, lufenuron, novaluron, noviflumuron and triflumuron; juvenile hormone mimics such as diofenolan, fenoxycarb, methoprene and pyriproxyfen; octopamine receptor ligands such as amitraz; molting inhibitors and ecdysone agonists such as azadirachtin, methoxyfenozide and tebufenozide; ryanodine receptor ligands such as ryanodine, anthranilic diamides such as chlorantraniliprole (see U.S. Pat. No. 6,747,047, PCT Publications WO 2003/015518 and WO 2004/067528) and flubendiamide (see U.S. Pat. No. 6,603,044); nereistoxin analogs such as cartap; mitochondrial electron transport inhibitors such as chlorfenapyr, hydramethylnon and pyridaben; lipid biosynthesis inhibitors such as spirodiclofen and spiromesifen; cyclodiene insecticides such as dieldrin or endosulfan; pyrethroids; carbamates; insecticidal ureas; and biological agents including nucleopolyhedro viruses (NPV), members of Bacillus thuringiensis, encapsulated delta-endotoxins of Bacillus thuringiensis, and other naturally occurring or genetically modified insecticidal viruses.

Further examples of biologically active compounds or agents with which compounds of this invention can be formulated are: fungicides such as 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, acibenzoiar, aldimorph, amisulbrom, azaconazole, azoxystrobin, benalaxyl, benomyl, benthiavalicarb, benthiavalicarb-isopropyl, binomial, biphenyl, bitertanol, blasticidin-S, Bordeaux mixture (Tribasic copper sulfate), boscalid/nicobifen, bromuconazole, bupirimate, buthiobate, carboxin, carpropamid, captafol, captan, carbendazim, chloroneb, chlorothalonil, chlozolinate, clotrimazole, copper oxychloride, copper salts such as copper sulfate and copper hydroxide, cyazofamid, cyflunamid, cymoxanil, cyproconazole, cyprodinil, dichlofluanid, diclocymet, diclomezine, dicloran, diethofencarb, difenoconazole, dimethomorph, dimoxystrobin, diniconazole, diniconazole-M, dinocap, discostrobin, dithianon, dodemorph, dodine, econazole, etaconazole, edifenphos, epoxiconazole, ethaboxam, ethirimol, ethridiazole, famoxadone, fenamidone, fenarimol, fenbuconazole, fencaramid, fenfuram, fenhexamide, fenoxanil, fenpiclonil, fenpropidin, fenpropimorph, fentin acetate, fentin hydroxide, ferbam, ferfurazoate, ferimzone, fluazinam, fludioxonil, flumetover, fluopicolide, fluoxastrobin, fluquinconazole, fluquinconazole, fusilazole, flusulfamide, flutolanil, flutriafol, fluxapyroxad, folpet, fosetyl-aluminum, fthalide, fuberidazole, furalaxyl, furametpyr, hexaconazole, hymexazole, guazatine, imazalil, imibenconazole, iminoctadine, iodicarb, ipconazole, iprobenfos, iprodione, iprovalicarb, isoconazole, isoprothiolane, isotianil, kasugamycin, kresoxim-methyl, mancozeb, mandipropamid, maneb, mapanipyrin, mefenoxam, mepronil, metalaxyl, metconazole, methasulfocarb, metiram, metominostrobin/fenominostrobin, mepanipyrim, metrafenone, miconazole, myclobutanil, neo-asozin (ferric methanearsonate), nuarimol, octhilinone, ofurace, orysastrobin, oxadixyl, oxolinic acid, oxpoconazole, oxycarboxin, paclobutrazol, penconazole, pencycuron, penflufen, penthiopyrad, perfurazoate, phosphonic acid, phthalide, picobenzamid, picoxystrobin, polyoxin, probenazole, prochloraz, procymidone, propamocarb, propamocarb-hydrochloride, propiconazole, propineb, proquinazid, prothioconazole, pyraclostrobin, pyrametostrobin, pyraoxystrobin, pryazophos, pyrifenox, pyrimethanil, pyrifenox, pyriofenone, pyrolnitrine, pyroquilon, quinconazole, quinoxyfen, quintozene, silthiofam, simeconazole, spiroxamine, streptomycin, sulfur, tebuconazole, tebufloquin, techrazene, tecloftalam, tecnazene, tetraeonazole, thiabendazole, thifluzamide, thiophanate, thiophanate-methyl, thiram, tiadinil, tolclofos-methyl, tolyfluanid, triadimefon, triadimenol, triarimol, triazoxide, tridemorph, trimorphamide, tricyclazole, trifloxystrobin, triforine, triticonazole, uniconazole, validamycin, valifenalate, vinclozolin, zineb, ziram, and zoxamide; nematocides such as aldicarb, imicyafos, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad.

Of note are fungicides and compositions comprising fungicides such as 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, azoxystrobin, copper hydroxide, cymoxanil, cyproconazole, difenoconazole, famoxadone, fenoxanil, ferimzone, fusilazole, flutolanil, fthalide, furametpyr, hexaconazole, isoprothiolane, isotianil, kasugamycin, mancozeb, metominostrobin, orysastrobin, pencycuron, penthiopyrad, picoxystrobin, probenazole, propiconazole, proquinazid, pyroquilon, simeconazole, tiadinil, tricyclazole, trifloxystrobin and validamycin.

In certain instances, combinations of a compound of this invention with other biologically active (particularly invertebrate pest control) compounds or agents (i.e. active ingredients) can result in a greater-man-additive (i.e. synergistic) effect. Reducing the quantity of active ingredients released, in the environment while ensuring effective pest control is always desirable. When synergism of invertebrate pest control active ingredients occurs at application rates giving agronomically satisfactory levels of invertebrate pest control, such combinations can be advantageous for reducing crop production cost and decreasing environmental load.

Compounds of this invention and compositions thereof can be applied to plants genetically transformed to express proteins toxic to invertebrate pests (such as Bacillus thuringiensis delta-endotoxins). Such an application may provide a broader spectrum of plant protection and be advantageous for resistance management. The effect of the exogenousiy applied, invertebrate pest control compounds of this invention may be synergistic with the expressed toxin proteins.

General references for these agricultural protectants (i.e. insecticides, fungicides, nematocides, acaricides, herbicides and biological agents) include The Pesticide Manual, 13th Edition, C, D. S. Tomlin, Ed., British Crop Protection Council, Farnham, Surrey, U.K., 2003 and The BioPesticide Manual, 2^{nd} Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1, an N-oxide or salt thereof, is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired, spectrum of biological activity. It will be evident that including these additional components can expand the spectrum of invertebrate pests controlled beyond the spectrum controlled by the compound of Formula 1 alone.

Table A lists specific combinations of a compound of Formula 1 with other invertebrate pest control agents illustrative of the mixtures, compositions and methods of the present invention. The first column of Table A lists the specific invertebrate pest control agents (e.g., “Abamectin” in the first line). The second column of Table A lists the mode of action (if known) or chemical class of the invertebrate pest control agents. The third column of Table A lists embodiment(s) of ranges of weight ratios for rates at which a compound of Formula 1 can be applied relative to an invertebrate pest control agent (e.g., “50:1 to 1:50” of a compound, of Formula 1 relative to abamectin by weight). Thus, for example, the first line of Table A specifically discloses the combination of a compound of Formula 1 with abamectin can be applied in a weight ratio between 50:1 to 1:50. The remaining lines of Table A are to be construed similarly. Of further note Table A lists specific combinations of a compound of Formula 1 with other invertebrate pest control agents illustrative of the mixtures, compositions and methods of the present invention and includes additional embodiments of weight ratio ranges for application rates.

TABLE A

| Invertebrate Pest | Mode of Action or | Typical |
|---|---|---|
| Control Agent | Chemical Class | Weight Ratio |
| Abamectin | macrocyclic lactones | 50:1 to 1:50 |
| Acetamiprid | neonicotinoids | 150:1 to 1:200 |
| Amitraz | octopamine receptor ligands | 200:1 to 1:100 |
| Avermectin | macrocyclic lactones | 50:1 to 1:50 |
| Azadirachtin | ecdysone agonists | 100:1 to 1:120 |
| Beta-cyfluthrin | sodium channel modulators | 150:1 to 1:200 |
| Bifenthrin | sodium channel modulators | 100:1 to 1:10 |
| Buprofezin | chitin synthesis inhibitors | 500:1 to 1:50 |
| Cartap | nereistoxin analogs | 100:1 to 1:200 |
| Chlorantraniliprole | ryanodine receptor ligands | 100:1 to 1:120 |
| Chlorfenapyr | mitochondrial electron | 300:1 to 1:200 |
| | transport inhibitors | |
| Chlorpyrifos | cholinesterase inhibitors | 500:1 to 1:200 |
| Clothianidin | neonicotinoids | 100:1 to 1:400 |
| Cyfluthrin | sodium channel modulators | 150:1 to 1:200 |
| Cyhalothrin | sodium channel modulators | 150:1 to 1:200 |
| Cypermethrin | sodium channel modulators | 150:1 to 1:200 |
| Cyromazine | chitin synthesis inhibitors | 400:1 to 1:50 |
| Deltamethrin | sodium channel modulators | 50:1 to 1:400 |
| Dieldrin | cyclodiene insecticides | 200:1 to 1:100 |
| Dinotefuran | neonicotinoids | 150:1 to 1:200 |
| Diofenolan | molting inhibitor | 150:1 to 1:200 |
| Emamectin | macrocyclic lactones | 50:1 to 1:10 |
| Endosulfan | cyclodiene insecticides | 200:1 to 1:100 |
| Esfenvalerate | sodium channel modulators | 100:1 to 1:400 |
| Ethiprole | GABA-regulated chloride | 200:1 to 1:100 |
| | channel blockers | |
| Fenothiocarb | | 150:1 to 1:200 |
| Fenoxycarb | juvenile hormone mimics | 500:1 to 1:100 |
| Fenvalerate | sodium channel modulators | 150:1 to 1:200 |
| Fipronil | GABA-regulated chloride | 150:1 to 1:100 |
| | channel blockers | |
| Flonicamid | | 200:1 to 1:100 |
| Flubendiamide | ryanodine receptor ligands | 100:1 to 1:120 |
| Flufenoxuron | chitin synthesis inhibitors | 200:1 to 1:100 |
| Hexaflumuron | chitin synthesis inhibitors | 300:1 to 1:50 |
| Hydramethylnon | mitochondrial electron | 150:1 to 1:250 |
| | transport inhibitors | |
| Imidacloprid | neonicotinoids | 1000:1 to 1:1000 |
| Indoxacarb | sodium channel modulators | 200:1 to 1:50 |
| Lambda-cyhalothrin | sodium channel modulators | 50:1 to 1:250 |
| Lufenuron | chitin synthesis inhibitors | 500:1 to 1:250 |
| Metaflumizone | | 200:1 to 1:200 |
| Methomyl | cholinesterase inhibitors | 500:1 to 1:100 |
| Methoprene | juvenile hormone mimics | 500:1 to 1:100 |
| Methoxyfenozide | ecdysone agonists | 50:1 to 1:50 |
| Nitenpyram | neonicotinoids | 150:1 to 1:200 |
| Nithiazine | neonicotinoids | 150:1 to 1:200 |
| Novaluron | chitin synthesis inhibitors | 500:1 to 1:150 |
| Oxamyl | cholinesterase inhibitors | 200:1 to 1:200 |
| Pymetrozine | | 200:1 to 1:100 |
| Pyrethrin | sodium channel modulators | 100:1 to 1:10 |
| Pyridaben | mitochondrial electron | 200:1 to 1:100 |
| | transport inhibitors | |
| Pyridalyl | | 200:1 to 1:100 |
| Pyriproxyfen | juvenile hormone mimics | 500:1 to 1:100 |
| Ryanodine | ryanodine receptor ligands | 100:1 to 1:120 |
| Spinetoram | macrocyclic lactones | 150:1 to 1:100 |
| Spinosad | macrocyclic lactones | 500:1 to 1:10 |
| Spirodiclofen | lipid biosynthesis inhibitors | 200:1 to 1:200 |
| Spiromesifen | lipid biosynthesis inhibitors | 200:1 to 1:200 |
| Tebufenozide | ecdysone agonists | 500:1 to 1:250 |
| Thiacloprid | neonicotinoids | 100:1 to 1:200 |
| Thiamethoxam | neonicotinoids | 1250:1 to 1:1000 |
| Thiodicarb | cholinesterase inhibitors | 500:1 to 1:400 |
| Thiosultap-sodium | | 150:1 to 1:100 |
| Tralomethrin | sodium channel modulators | 150:1 to 1:200 |
| Triazamate | cholinesterase inhibitors | 250:1 to 1:100 |
| Triflumuron | chitin synthesis inhibitors | 200:1 to 1:100 |
| Bacillus thuringiensis | biological agents | 50:1 to 1:10 |
| Bacillus thuringiensis | biological agents | 50:1 to 1:10 |
| delta-endotoxin | | |
| NPV (e.g., Gemstar) | biological agents | 50:1 to 1:10 |
| Cyantraniliprole | ryanodine receptor ligands | 100:1 to 1:120 |

Of note is the composition of the present invention wherein the at least one additional biologically active compound or agent is selected from the Invertebrate Pest Control Agents listed in Table A above.

The weight ratios of a compound, including a compound of Formula 1, an N-oxide or salt thereof, to the additional invertebrate pest control agent typically are between 1000:1 and 1:1000, with one embodiment being between 500:1 and 1:500, another embodiment being between 250:1 and 1:200 and another embodiment being between 100:1 and 1:50.

Listed below in Table B are embodiments of specific compositions comprising a compound of Formula 1 (compound numbers refer to compounds in Index Table A) and an additional invertebrate pest control agent.

TABLE B

| Mixture | Comp. | | Invertebrate Pest Control |
|---|---|---|---|
| No. | No. | and | Agent |
| A-1 | 43 | and | Abamectin |
| A-2 | 43 | and | Acetamiprid |
| A-3 | 43 | and | Amitraz |
| A-4 | 43 | and | Avermectin |
| A-5 | 43 | and | Azadirachtin |
| A-6 | 43 | and | Beta-cyfluthrin |
| A-7 | 43 | and | Bifenthrin |
| A-8 | 43 | and | Buprofezin |
| A-9 | 43 | and | Cartap |
| A-10 | 43 | and | Chlorantraniliprole |
| A-11 | 43 | and | Chlorfenapyr |
| A-12 | 43 | and | Chlorpyrifos |
| A-13 | 43 | and | Clothianidin |
| A-14 | 43 | and | Cyfluthrin |
| A-15 | 43 | and | Cyhalothrin |
| A-16 | 43 | and | Cypermethrin |
| A-17 | 43 | and | Cyromazine |
| A-18 | 43 | and | Deltamethrin |
| A-19 | 43 | and | Dieldrin |
| A-20 | 43 | and | Dinotefuran |
| A-21 | 43 | and | Diofenolan |
| A-22 | 43 | and | Emamectin |
| A-23 | 43 | and | Endosulfan |
| A-24 | 43 | and | Esfenvalerate |
| A-25 | 43 | and | Ethiprole |
| A-26 | 43 | and | Fenothiocarb |
| A-27 | 43 | and | Fenoxycarb |
| A-28 | 43 | and | Fenvalerate |
| A-29 | 43 | and | Fipronil |
| A-30 | 43 | and | Flonicamid |
| A-31 | 43 | and | Flubendiamide |
| A-32 | 43 | and | Flufenoxuron |
| A-33 | 43 | and | Hexaflumuron |
| A-34 | 43 | and | Hydramethylnon |
| A-35 | 43 | and | Imidacloprid |
| A-36 | 43 | and | Indoxacarb |
| A-37 | 43 | and | Lambda-cyhalothrin |
| A-38 | 43 | and | Lufenuron |
| A-39 | 43 | and | Metaflumizone |
| A-40 | 43 | and | Methomyl |
| A-41 | 43 | and | Methoprene |
| A-42 | 43 | and | Methoxyfenozide |
| A-43 | 43 | and | Nitenpyram |
| A-44 | 43 | and | Nithiazine |
| A-45 | 43 | and | Novaluron |
| A-46 | 43 | and | Oxamyl |
| A-47 | 43 | and | Pymetrozine |
| A-48 | 43 | and | Pyrethrin |
| A-49 | 43 | and | Pyridaben |
| A-50 | 43 | and | Pyridalyl |
| A-51 | 43 | and | Pyriproxyfen |
| A-52 | 43 | and | Ryanodine |
| A-53 | 43 | and | Spinetoram |
| A-54 | 43 | and | Spinosad |
| A-55 | 43 | and | Spirodiclofen |
| A-56 | 43 | and | Spiromesifen |
| A-57 | 43 | and | Tebufenozide |
| A-58 | 43 | and | Thiacloprid |
| A-59 | 43 | and | Thiamethoxam |
| A-60 | 43 | and | Thiodicarb |
| A-61 | 43 | and | Thiosultap-sodium |
| A-62 | 43 | and | Tralomethrin |
| A-63 | 43 | and | Triazamate |
| A-64 | 43 | and | Triflumuron |
| A-65 | 43 | and | Bacillus thuringiensis |
| A-66 | 43 | and | Bacillus thuringiensis |
| | | | delta-endotoxin |
| A-67 | 43 | and | NPV (e.g., Gemstar) |
| A-68 | 43 | and | Cyantraniliprole |
| B-1 | 87 | and | Abamectin |
| B-2 | 87 | and | Acetamiprid |
| B-3 | 87 | and | Amitraz |
| B-4 | 87 | and | Avermectin |
| B-5 | 87 | and | Azadirachtin |
| B-6 | 87 | and | Beta-cyfluthrin |
| B-7 | 87 | and | Bifenthrin |
| B-8 | 87 | and | Buprofezin |
| B-9 | 87 | and | Cartap |
| B-10 | 87 | and | Chlorantraniliprole |
| B-11 | 87 | and | Chlorfenapyr |
| B-12 | 87 | and | Chlorpyrifos |
| B-13 | 87 | and | Clothianidin |
| B-14 | 87 | and | Cyfluthrin |
| B-15 | 87 | and | Cyhalothrin |
| B-16 | 87 | and | Cypermethrin |
| B-17 | 87 | and | Cyromazine |
| B-18 | 87 | and | Deltamethrin |
| B-19 | 87 | and | Dieldrin |
| B-20 | 87 | and | Dinotefuran |
| B-21 | 87 | and | Diofenolan |
| B-22 | 87 | and | Emamectin |
| B-23 | 87 | and | Endosulfan |
| B-24 | 87 | and | Esfenvalerate |
| B-25 | 87 | and | Ethiprole |
| B-26 | 87 | and | Fenothiocarb |
| B-27 | 87 | and | Fenoxycarb |
| B-28 | 87 | and | Fenvalerate |
| B-29 | 87 | and | Fipronil |
| B-30 | 87 | and | Flonicamid |
| B-31 | 87 | and | Flubendiamide |
| B-32 | 87 | and | Flufenoxuron |
| B-33 | 87 | and | Hexaflumuron |
| B-34 | 87 | and | Hydramethylnon |
| B-35 | 87 | and | Imidacloprid |
| B-36 | 87 | and | Indoxacarb |
| B-37 | 87 | and | Lambda-cyhalothrin |
| B-38 | 87 | and | Lufenuron |
| B-39 | 87 | and | Metaflumizone |
| B-40 | 87 | and | Methomyl |
| B-41 | 87 | and | Methoprene |
| B-42 | 87 | and | Methoxyfenozide |
| B-43 | 87 | and | Nitenpyram |
| B-44 | 87 | and | Nithiazine |
| B-45 | 87 | and | Novaluron |
| B-46 | 87 | and | Oxamyl |
| B-47 | 87 | and | Pymetrozine |
| B-48 | 87 | and | Pyrethrin |
| B-49 | 87 | and | Pyridaben |
| B-50 | 87 | and | Pyridalyl |
| B-51 | 87 | and | Pyriproxyfen |
| B-52 | 87 | and | Ryanodine |
| B-53 | 87 | and | Spinetoram |
| B-54 | 87 | and | Spinosad |
| B-55 | 87 | and | Spirodiclofen |
| B-56 | 87 | and | Spiromesifen |
| B-57 | 87 | and | Tebufenozide |
| B-58 | 87 | and | Thiacloprid |
| B-59 | 87 | and | Thiamethoxam |
| B-60 | 87 | and | Thiodicarb |
| B-61 | 87 | and | Thiosultap-sodium |
| B-62 | 87 | and | Tralomethrin |
| B-63 | 87 | and | Triazamate |
| B-64 | 87 | and | Triflumuron |
| B-65 | 87 | and | Bacillus thuringiensis |
| B-66 | 87 | and | Bacillus thuringiensis |
| | | | delta-endotoxin |
| B-67 | 87 | and | NPV (e.g., Gemstar) |
| B-68 | 87 | and | Cyantraniliprole |
| C-1 | 92 | and | Abamectin |
| C-2 | 92 | and | Acetamiprid |
| C-3 | 92 | and | Amitraz |
| C-4 | 92 | and | Avermectin |
| C-5 | 92 | and | Azadirachtin |
| C-6 | 92 | and | Beta-cyfluthrin |
| C-7 | 92 | and | Bifenthrin |
| C-8 | 92 | and | Buprofezin |
| C-9 | 92 | and | Cartap |
| C-10 | 92 | and | Chlorantraniliprole |
| C-11 | 92 | and | Chlorfenapyr |
| C-12 | 92 | and | Chlorpyrifos |
| C-13 | 92 | and | Clothianidin |
| C-14 | 92 | and | Cyfluthrin |
| C-15 | 92 | and | Cyhalothrin |
| C-16 | 92 | and | Cypermethrin |
| C-17 | 92 | and | Cyromazine |
| C-18 | 92 | and | Deltamethrin |
| C-19 | 92 | and | Dieldrin |
| C-20 | 92 | and | Dinotefuran |
| C-21 | 92 | and | Diofenolan |
| C-22 | 92 | and | Emamectin |
| C-23 | 92 | and | Endosulfan |
| C-24 | 92 | and | Esfenvalerate |
| C-25 | 92 | and | Ethiprole |
| C-26 | 92 | and | Fenothiocarb |
| C-27 | 92 | and | Fenoxycarb |
| C-28 | 92 | and | Fenvalerate |
| C-29 | 92 | and | Fipronil |
| C-30 | 92 | and | Flonicamid |
| C-31 | 92 | and | Flubendiamide |
| C-32 | 92 | and | Flufenoxuron |
| C-33 | 92 | and | Hexaflumuron |
| C-34 | 92 | and | Hydramethylnon |
| C-35 | 92 | and | Imidacloprid |
| C-36 | 92 | and | Indoxacarb |
| C-37 | 92 | and | Lambda-cyhalothrin |
| C-38 | 92 | and | Lufenuron |
| C-39 | 92 | and | Metaflumizone |
| C-40 | 92 | and | Methomyl |
| C-41 | 92 | and | Methoprene |
| C-42 | 92 | and | Methoxyfenozide |
| C-43 | 92 | and | Nitenpyram |
| C-44 | 92 | and | Nithiazine |
| C-45 | 92 | and | Novaluron |
| C-46 | 92 | and | Oxamyl |
| C-47 | 92 | and | Pymetrozine |
| C-48 | 92 | and | Pyrethrin |
| C-49 | 92 | and | Pyridaben |
| C-50 | 92 | and | Pyridalyl |
| C-51 | 92 | and | Pyriproxyfen |
| C-52 | 92 | and | Ryanodine |
| C-53 | 92 | and | Spinetoram |
| C-54 | 92 | and | Spinosad |
| C-55 | 92 | and | Spirodiclofen |
| C-56 | 92 | and | Spiromesifen |
| C-57 | 92 | and | Tebufenozide |
| C-58 | 92 | and | Thiacloprid |
| C-59 | 92 | and | Thiamethoxam |
| C-60 | 92 | and | Thiodicarb |
| C-61 | 92 | and | Thiosultap-sodium |
| C-62 | 92 | and | Tralomethrin |
| C-63 | 92 | and | Triazamate |
| C-64 | 92 | and | Triflumuron |
| C-65 | 92 | and | Bacillus thuringiensis |
| C-66 | 92 | and | Bacillus thuringiensis |
| | | | delta-endotoxin |
| C-67 | 92 | and | NPV (e.g., Gemstar) |
| C-68 | 92 | and | Cyantraniliprole |
| D-1 | 88 | and | Abamectin |
| D-2 | 88 | and | Acetamiprid |
| D-3 | 88 | and | Amitraz |
| D-4 | 88 | and | Avermectin |
| D-5 | 88 | and | Azadirachtin |
| D-6 | 88 | and | Beta-cyfluthrin |
| D-7 | 88 | and | Bifenthrin |
| D-8 | 88 | and | Buprofezin |
| D-9 | 88 | and | Cartap |
| D-10 | 88 | and | Chlorantraniliprole |
| D-11 | 88 | and | Chlorfenapyr |
| D-12 | 88 | and | Chlorpyrifos |
| D-13 | 88 | and | Clothianidin |
| D-14 | 88 | and | Cyfluthrin |
| D-15 | 88 | and | Cyhalothrin |
| D-16 | 88 | and | Cypermethrin |
| D-17 | 88 | and | Cyromazine |
| D-18 | 88 | and | Deltamethrin |
| D-19 | 88 | and | Dieldrin |
| D-20 | 88 | and | Dinotefuran |
| D-21 | 88 | and | Diofenolan |
| D-22 | 88 | and | Emamectin |
| D-23 | 88 | and | Endosulfan |
| D-24 | 88 | and | Esfenvalerate |
| D-25 | 88 | and | Ethiprole |
| D-26 | 88 | and | Fenothiocarb |
| D-27 | 88 | and | Fenoxycarb |
| D-28 | 88 | and | Fenvalerate |
| D-29 | 88 | and | Fipronil |
| D-30 | 88 | and | Flonicamid |
| D-31 | 88 | and | Flubendiamide |
| D-32 | 88 | and | Flufenoxuron |
| D-33 | 88 | and | Hexaflumuron |
| D-34 | 88 | and | Hydramethylnon |
| D-35 | 88 | and | Imidacloprid |
| D-36 | 88 | and | Indoxacarb |
| D-37 | 88 | and | Lambda-cyhalothrin |
| D-38 | 88 | and | Lufenuron |
| D-39 | 88 | and | Metaflumizone |
| D-40 | 88 | and | Methomyl |
| D-41 | 88 | and | Methoprene |
| D-42 | 88 | and | Methoxyfenozide |
| D-43 | 88 | and | Nitenpyram |
| D-44 | 88 | and | Nithiazine |
| D-45 | 88 | and | Novaluron |
| D-46 | 88 | and | Oxamyl |
| D-47 | 88 | and | Pymetrozine |
| D-48 | 88 | and | Pyrethrin |
| D-49 | 88 | and | Pyridaben |
| D-50 | 88 | and | Pyridalyl |
| D-51 | 88 | and | Pyriproxyfen |
| D-52 | 88 | and | Ryanodine |
| D-53 | 88 | and | Spinetoram |
| D-54 | 88 | and | Spinosad |
| D-55 | 88 | and | Spirodiclofen |
| D-56 | 88 | and | Spiromesifen |
| D-57 | 88 | and | Tebufenozide |
| D-58 | 88 | and | Thiacloprid |
| D-59 | 88 | and | Thiamethoxam |
| D-60 | 88 | and | Thiodicarb |
| D-61 | 88 | and | Thiosultap-sodium |
| D-62 | 88 | and | Tralomethrin |
| D-63 | 88 | and | Triazamate |
| D-64 | 88 | and | Triflumuron |
| D-65 | 88 | and | Bacillus thuringiensis |
| D-66 | 88 | and | Bacillus thuringiensis |
| | | | delta-endotoxin |
| D-67 | 88 | and | NPV (e.g., Gemstar) |
| D-68 | 88 | and | Cyantraniliprole |
| E-1 | 93 | and | Abamectin |
| E-2 | 93 | and | Acetamiprid |
| E-3 | 93 | and | Amitraz |
| E-4 | 93 | and | Avermectin |
| E-5 | 93 | and | Azadirachtin |
| E-6 | 93 | and | Beta-cyfluthrin |
| E-7 | 93 | and | Bifenthrin |
| E-8 | 93 | and | Buprofezin |
| E-9 | 93 | and | Cartap |
| E-10 | 93 | and | Chlorantraniliprole |
| E-11 | 93 | and | Chlorfenapyr |
| E-12 | 93 | and | Chlorpyrifos |
| E-13 | 93 | and | Clothianidin |
| E-14 | 93 | and | Cyfluthrin |
| E-15 | 93 | and | Cyhalothrin |
| E-16 | 93 | and | Cypermethrin |
| E-17 | 93 | and | Cyromazine |
| E-18 | 93 | and | Deltamethrin |
| E-19 | 93 | and | Dieldrin |
| E-20 | 93 | and | Dinotefuran |
| E-21 | 93 | and | Diofenolan |
| E-22 | 93 | and | Emamectin |
| E-23 | 93 | and | Endosulfan |
| E-24 | 93 | and | Esfenvalerate |
| E-25 | 93 | and | Ethiprole |
| E-26 | 93 | and | Fenothiocarb |
| E-27 | 93 | and | Fenoxycarb |
| E-28 | 93 | and | Fenvalerate |
| E-29 | 93 | and | Fipronil |
| E-30 | 93 | and | Flonicamid |
| E-31 | 93 | and | Flubendiamide |
| E-32 | 93 | and | Flufenoxuron |
| E-33 | 93 | and | Hexaflumuron |
| E-34 | 93 | and | Hydramethylnon |
| E-35 | 93 | and | Imidacloprid |
| E-36 | 93 | and | Indoxacarb |
| E-37 | 93 | and | Lambda-cyhalothrin |
| E-38 | 93 | and | Lufenuron |
| E-39 | 93 | and | Metaflumizone |
| E-40 | 93 | and | Methomyl |
| E-41 | 93 | and | Methoprene |
| E-42 | 93 | and | Methoxyfenozide |
| E-43 | 93 | and | Nitenpyram |
| E-44 | 93 | and | Nithiazine |
| E-45 | 93 | and | Novaluron |
| E-46 | 93 | and | Oxamyl |
| E-47 | 93 | and | Pymetrozine |
| E-48 | 93 | and | Pyrethrin |
| E-49 | 93 | and | Pyridaben |
| E-50 | 93 | and | Pyridalyl |
| E-51 | 93 | and | Pyriproxyfen |
| E-52 | 93 | and | Ryanodine |
| E-53 | 93 | and | Spinetoram |
| E-54 | 93 | and | Spinosad |
| E-55 | 93 | and | Spirodiclofen |
| E-56 | 93 | and | Spiromesifen |
| E-57 | 93 | and | Tebufenozide |
| E-58 | 93 | and | Thiacloprid |
| E-59 | 93 | and | Thiamethoxam |
| E-60 | 93 | and | Thiodicarb |
| E-61 | 93 | and | Thiosultap-sodium |
| E-62 | 93 | and | Tralomethrin |
| E-63 | 93 | and | Triazamate |
| E-64 | 93 | and | Triflumuron |
| E-65 | 93 | and | Bacillus thuringiensis |
| E-66 | 93 | and | Bacillus thuringiensis |
| | | | delta-endotoxin |
| E-67 | 93 | and | NPV (e.g., Gemstar) |
| E-68 | 93 | and | Cyantraniliprole |
| F-1 | 102 | and | Abamectin |
| F-2 | 102 | and | Acetamiprid |
| F-3 | 102 | and | Amitraz |
| F-4 | 102 | and | Avermectin |
| F-5 | 102 | and | Azadirachtin |
| F-6 | 102 | and | Beta-cyfluthrin |
| F-7 | 102 | and | Bifenthrin |
| F-8 | 102 | and | Buprofezin |
| F-9 | 102 | and | Cartap |
| F-10 | 102 | and | Chlorantraniliprole |
| F-11 | 102 | and | Chlorfenapyr |
| F-12 | 102 | and | Chlorpyrifos |
| F-13 | 102 | and | Clothianidin |
| F-14 | 102 | and | Cyfluthrin |
| F-15 | 102 | and | Cyhalothrin |
| F-16 | 102 | and | Cypermethrin |
| F-17 | 102 | and | Cyromazine |
| F-18 | 102 | and | Deltamethrin |
| F-19 | 102 | and | Dieldrin |
| F-20 | 102 | and | Dinotefuran |
| F-21 | 102 | and | Diofenolan |
| F-22 | 102 | and | Emamectin |
| F-23 | 102 | and | Endosulfan |
| F-24 | 102 | and | Esfenvalerate |
| F-25 | 102 | and | Ethiprole |
| F-26 | 102 | and | Fenothiocarb |
| F-27 | 102 | and | Fenoxycarb |
| F-28 | 102 | and | Fenvalerate |
| F-29 | 102 | and | Fipronil |
| F-30 | 102 | and | Flonicamid |
| F-31 | 102 | and | Flubendiamide |
| F-32 | 102 | and | Flufenoxuron |
| F-33 | 102 | and | Hexaflumuron |
| F-34 | 102 | and | Hydramethylnon |
| F-35 | 102 | and | Imidacloprid |
| F-36 | 102 | and | Indoxacarb |
| F-37 | 102 | and | Lambda-cyhalothrin |
| F-38 | 102 | and | Lufenuron |
| F-39 | 102 | and | Metaflumizone |
| F-40 | 102 | and | Methomyl |
| F-41 | 102 | and | Methoprene |
| F-42 | 102 | and | Methoxyfenozide |
| F-43 | 102 | and | Nitenpyram |
| F-44 | 102 | and | Nithiazine |
| F-45 | 102 | and | Novaluron |
| F-46 | 102 | and | Oxamyl |
| F-47 | 102 | and | Pymetrozine |
| F-48 | 102 | and | Pyrethrin |
| F-49 | 102 | and | Pyridaben |
| F-50 | 102 | and | Pyridalyl |
| F-51 | 102 | and | Pyriproxyfen |
| F-52 | 102 | and | Ryanodine |
| F-53 | 102 | and | Spinetoram |
| F-54 | 102 | and | Spinosad |
| F-55 | 102 | and | Spirodiclofen |
| F-56 | 102 | and | Spiromesifen |
| F-57 | 102 | and | Tebufenozide |
| F-58 | 102 | and | Thiacloprid |
| F-59 | 102 | and | Thiamethoxam |
| F-60 | 102 | and | Thiodicarb |
| F-61 | 102 | and | Thiosultap-sodium |
| F-62 | 102 | and | Tralomethrin |
| F-63 | 102 | and | Triazamate |
| F-64 | 102 | and | Triflumuron |
| F-65 | 102 | and | Bacillus thuringiensis |
| F-66 | 102 | and | Bacillus thuringiensis |
| | | | delta-endotoxin |
| F-67 | 102 | and | NPV (e.g., Gemstar) |
| F-68 | 102 | and | Cyantraniliprole |
| G-1 | 105 | and | Abamectin |
| G-2 | 105 | and | Acetamiprid |
| G-3 | 105 | and | Amitraz |
| G-4 | 105 | and | Avermectin |
| G-5 | 105 | and | Azadirachtin |
| G-6 | 105 | and | Beta-cyfluthrin |
| G-7 | 105 | and | Bifenthrin |
| G-8 | 105 | and | Buprofezin |
| G-9 | 105 | and | Cartap |
| G-10 | 105 | and | Chlorantraniliprole |
| G-11 | 105 | and | Chlorfenapyr |
| G-12 | 105 | and | Chlorpyrifos |
| G-13 | 105 | and | Clothianidin |
| G-14 | 105 | and | Cyfluthrin |
| G-15 | 105 | and | Cyhalothrin |
| G-16 | 105 | and | Cypermethrin |
| G-17 | 105 | and | Cyromazine |
| G-18 | 105 | and | Deltamethrin |
| G-19 | 105 | and | Dieldrin |
| G-20 | 105 | and | Dinotefuran |
| G-21 | 105 | and | Diofenolan |
| G-22 | 105 | and | Emamectin |
| G-23 | 105 | and | Endosulfan |
| G-24 | 105 | and | Esfenvalerate |
| G-25 | 105 | and | Ethiprole |
| G-26 | 105 | and | Fenothiocarb |
| G-27 | 105 | and | Fenoxycarb |
| G-28 | 105 | and | Fenvalerate |
| G-29 | 105 | and | Fipronil |
| G-30 | 105 | and | Flonicamid |
| G-31 | 105 | and | Flubendiamide |
| G-32 | 105 | and | Flufenoxuron |
| G-33 | 105 | and | Hexaflumuron |
| G-34 | 105 | and | Hydramethylnon |
| G-35 | 105 | and | Imidacloprid |
| G-36 | 105 | and | Indoxacarb |
| G-37 | 105 | and | Lambda-cyhalothrin |
| G-38 | 105 | and | Lufenuron |
| G-39 | 105 | and | Metaflumizone |
| G-40 | 105 | and | Methomyl |
| G-41 | 105 | and | Methoprene |
| G-42 | 105 | and | Methoxyfenozide |
| G-43 | 105 | and | Nitenpyram |
| G-44 | 105 | and | Nithiazine |
| G-45 | 105 | and | Novaluron |
| G-46 | 105 | and | Oxamyl |
| G-47 | 105 | and | Pymetrozine |
| G-48 | 105 | and | Pyrethrin |
| G-49 | 105 | and | Pyridaben |
| G-50 | 105 | and | Pyridalyl |
| G-51 | 105 | and | Pyriproxyfen |
| G-52 | 105 | and | Ryanodine |
| G-53 | 105 | and | Spinetoram |
| G-54 | 105 | and | Spinosad |
| G-55 | 105 | and | Spirodiclofen |
| G-56 | 105 | and | Spiromesifen |
| G-57 | 105 | and | Tebufenozide |
| G-58 | 105 | and | Thiacloprid |
| G-59 | 105 | and | Thiamethoxam |
| G-60 | 105 | and | Thiodicarb |
| G-61 | 105 | and | Thiosultap-sodium |
| G-62 | 105 | and | Tralomethrin |
| G-63 | 105 | and | Triazamate |
| G-64 | 105 | and | Triflumuron |
| G-65 | 105 | and | Bacillus thuringiensis |
| G-66 | 105 | and | Bacillus thuringiensis |
| | | | delta-endotoxin |
| G-67 | 105 | and | NPV (e.g., Gemstar) |
| G-68 | 105 | and | Cyantraniliprole |
| H-1 | 107 | and | Abamectin |
| H-2 | 107 | and | Acetamiprid |
| H-3 | 107 | and | Amitraz |
| H-4 | 107 | and | Avermectin |
| H-5 | 107 | and | Azadirachtin |
| H-6 | 107 | and | Beta-cyfluthrin |
| H-7 | 107 | and | Bifenthrin |
| H-8 | 107 | and | Buprofezin |
| H-9 | 107 | and | Cartap |
| H-10 | 107 | and | Chlorantraniliprole |
| H-11 | 107 | and | Chlorfenapyr |
| H-12 | 107 | and | Chlorpyrifos |
| H-13 | 107 | and | Clothianidin |
| H-14 | 107 | and | Cyfluthrin |
| H-15 | 107 | and | Cyhalothrin |
| H-16 | 107 | and | Cypermethrin |
| H-17 | 107 | and | Cyromazine |
| H-18 | 107 | and | Deltamethrin |
| H-19 | 107 | and | Dieldrin |
| H-20 | 107 | and | Dinotefuran |
| H-21 | 107 | and | Diofenolan |
| H-22 | 107 | and | Emamectin |
| H-23 | 107 | and | Endosulfan |
| H-24 | 107 | and | Esfenvalerate |
| H-25 | 107 | and | Ethiprole |
| H-26 | 107 | and | Fenothiocarb |
| H-27 | 107 | and | Fenoxycarb |
| H-28 | 107 | and | Fenvalerate |
| H-29 | 107 | and | Fipronil |
| H-30 | 107 | and | Flonicamid |
| H-31 | 107 | and | Flubendiamide |
| H-32 | 107 | and | Flufenoxuron |
| H-33 | 107 | and | Hexaflumuron |
| H-34 | 107 | and | Hydramethylnon |
| H-35 | 107 | and | Imidacloprid |
| H-36 | 107 | and | Indoxacarb |
| H-37 | 107 | and | Lambda-cyhalothrin |
| H-38 | 107 | and | Lufenuron |
| H-39 | 107 | and | Metaflumizone |
| H-40 | 107 | and | Methomyl |
| H-41 | 107 | and | Methoprene |
| H-42 | 107 | and | Methoxyfenozide |
| H-43 | 107 | and | Nitenpyram |
| H-44 | 107 | and | Nithiazine |
| H-45 | 107 | and | Novaluron |
| H-46 | 107 | and | Oxamyl |
| H-47 | 107 | and | Pymetrozine |
| H-48 | 107 | and | Pyrethrin |
| H-49 | 107 | and | Pyridaben |
| H-50 | 107 | and | Pyridalyl |
| H-51 | 107 | and | Pyriproxyfen |
| H-52 | 107 | and | Ryanodine |
| H-53 | 107 | and | Spinetoram |
| H-54 | 107 | and | Spinosad |
| H-55 | 107 | and | Spirodiclofen |
| H-56 | 107 | and | Spiromesifen |
| H-57 | 107 | and | Tebufenozide |
| H-58 | 107 | and | Thiacloprid |
| H-59 | 107 | and | Thiamethoxam |
| H-60 | 107 | and | Thiodicarb |
| H-61 | 107 | and | Thiosultap-sodium |
| H-62 | 107 | and | Tralomethrin |
| H-63 | 107 | and | Triazamate |
| H-64 | 107 | and | Triflumuron |
| H-65 | 107 | and | Bacillus thuringiensis |
| H-66 | 107 | and | Bacillus thuringiensis |
| | | | delta-endotoxin |
| H-67 | 107 | and | NPV (e.g., Gemstar) |
| H-68 | 107 | and | Cyantraniliprole |

The specific mixtures listed in Table B typically combine a compound of Formula 1 with the other invertebrate pest agent in the ratios specified in Table A.

Invertebrate pests are controlled in agronomic and nonagronomic applications by applying one or more compounds of this invention, typically in the form of a composition, in a biologically effective amount, to the environment of the pests, including the agronomic and/or nonagronomic locus of infestation, to the area to be protected, or directly on the pests to be controlled.

Thus the present invention comprises a method for controlling an invertebrate pest in agronomic and/or nonagronomic applications, comprising contacting the invertebrate pest or its environment with a biologically effective amount of one or more of the compounds of the invention, or with a composition comprising at least one such compound or a composition comprising at least one such compound and a biologically effective amount of at least one additional biologically active compound or agent. Examples of suitable compositions comprising a compound of the invention and a biologically effective amount of at least one additional biologically active compound or agent include granular compositions wherein the additional active compound is present on the same granule as the compound of the invention or on granules separate from those of the compound of the invention.

Embodiments of the method of this invention include contacting the environment. Of note is the method wherein the environment is a plant. Also of note is the method wherein the environment is an animal. Also of note is the method wherein the environment is a seed.

To achieve contact with a compound or composition of the invention to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a compound of the invention can be applied to the plant foliage or the soil. Compounds of this invention can also be effectively delivered, through plant uptake by contacting the plant with a composition comprising a compound of this invention applied as a soil drench of a liquid formulation, a granular formulation to the soil, a nursery box treatment or a dip of transplants. Of note is a composition of the present invention in the form of a soil drench liquid formulation. Also of note is a method for controlling an invertebrate pest comprising contacting the invertebrate pest or its environment with a biologically effective amount of a compound of the present invention or with a composition comprising a biologically effective amount of a compound of the present invention. Of further note is this method wherein the environment is soil and the composition is applied to the soil as a soil drench formulation. Of further note is that compounds of this invention are also effective by localized application to the locus of infestation. Other methods of contact include application of a compound or a composition of the invention by direct and residual sprays, aerial sprays, gels, seed coatings, microencapsulations, systemic uptake, baits, ear tags, boluses, loggers, fumigants, aerosols, dusts and many others. One embodiment of a method of contact is a dimensionally stable fertilizer granule, stick or tablet comprising a compound or composition of the invention. The compounds of this invention can also be impregnated into materials for fabricating invertebrate control devices (e.g., insect netting).

Compounds of this invention are also useful in seed treatments for protecting seeds from invertebrate pests. In the context of the present disclosure and claims, treating a seed means contacting the seed with a biologically effective amount of a compound of this invention, which is typically formulated as a composition of the invention. This seed, treatment protects the seed from invertebrate soil pests and generally can also protect roots and other plant parts in contact with the soil of the seedling developing from the germinating seed. The seed, treatment may also provide protection of foliage by translocation of the compound of this invention or a second active ingredient within the developing plant. Seed treatments can be applied to all types of seeds, including those from which plants genetically transformed to express specialized, traits will germinate. Representative examples include those expressing proteins toxic to invertebrate pests, such as Bacillus thuringiensis toxin or those expressing herbicide resistance such as glyphosate acetyltransferase, which provides resistance to glyphosate.

One method of seed treatment is by spraying or dusting the seed with a compound of the invention (i.e. as a formulated, composition) before sowing the seeds. Compositions formulated for seed treatment generally comprise a film former or adhesive agent. Therefore typically a seed coating composition of the present invention comprises a biologically effective amount of a compound of Formula 1, an N-oxide or salt thereof, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment Progress and Prospects, 1994 BCPC Mongraph No. 57, and references listed therein.

The treated seed typically comprises a compound of the present invention in an amount from about 0.1 g to 1 kg per 100 kg of seed (i.e. from about 0.0001 to 1% by weight of the seed before treatment). A flowable suspension formulated for seed treatment typically comprises from about 0.5 to about 70% of the active ingredient, from about 0.5 to about 30%) of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention can be incorporated into a bait composition that is consumed by an invertebrate pest or used within a device such as a trap, bait station, and the like. Such a bait composition can be in the form of granules which comprise (a) active ingredients, namely a biologically effective amount of a compound of Formula 1, an N-oxide, or salt thereof; (b) one or more food materials: optionally (c) an attractant, and optionally (d) one or more humectants. Of note are granules or bait compositions which comprise between about 0.001-5% active ingredients, about 40-99% food material and/or attractant; and optionally about 0.05-10% humectants, which are effective in controlling soil invertebrate pests at very low application rates, particularly at doses of active ingredient that are lethal by ingestion rather than by direct contact. Some food materials can function both as a food source and an attractant. Food materials include carbohydrates, proteins and lipids. Examples of food materials are vegetable flour, sugar, starches, animal fat, vegetable oil, yeast extracts and milk solids. Examples of attractants are odorants and flavorants, such as fruit or plant extracts, perfume, or other animal or plant component, pheromones or other agents known to attract a target invertebrate pest. Examples of humectants, i.e. moisture retaining agents, are glycols and other polyols, glycerine and sorbitol. Of note is a bait composition (and a method utilizing such a bait composition) used to control at least one invertebrate pest selected from the group consisting of ants, termites and cockroaches. A device for controlling an invertebrate pest can comprise the present bait composition and a housing adapted to receive the bait composition, wherein the housing has at least one opening sized to permit the invertebrate pest to pass through the opening so the invertebrate pest can gain access to the bait composition from a location outside the housing, and wherein the housing is further adapted to be placed in or near a locus of potential or known activity for the invertebrate pest.

The compounds of this invention can be applied without other adjuvants, but most often application will be of a formulation comprising one or more active ingredients with suitable carriers, diluents, and surfactants and possibly in combination with a food depending on the contemplated end use. One method, of application involves spraying a water dispersion or refined oil solution of a compound of the present invention. Combinations with spray oils, spray oil concentrations, spreader stickers, adjuvants, other solvents, and synergists such as piperonyl butoxide often enhance compound efficacy. For nonagronomic uses such sprays can be applied from spray containers such as a can, a bottle or other container, either by means of a pump or by releasing it from a pressurized container, e.g., a pressurized aerosol spray can. Such spray compositions can take various forms, for example, sprays, mists, foams, fumes or fog. Such spray compositions thus can further comprise propellants, foaming agents, etc. as needed for application. Of note is a spray composition comprising a biologically effective amount of a compound or a composition of the present invention and a carrier. One embodiment of such a spray composition comprises a biologically effective amount of a compound or a composition of the present invention and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one invertebrate pest selected from the group consisting of mosquitoes, black flies, stable flies, deer flies, horse flies, wasps, yellow jackets, hornets, ticks, spiders, ants, gnats, and the like, including individually or in combinations.

Nonagronomic uses refer to invertebrate pest control in the areas other than fields of crop plants. Nonagronomic uses of the present compounds and compositions include control of invertebrate pests in stored grains, beans and other foodstuffs, and in textiles such as clothing and carpets. Nonagronomic uses of the present compounds and compositions also include invertebrate pest control in ornamental plants, forests, in yards, along roadsides and railroad rights of way, and on turf such as lawns, golf courses and pastures. Nonagronomic uses of the present compounds and compositions also include invertebrate pest control in houses and other buildings which may be occupied by humans and/or companion, farm, ranch, zoo or other animals. Nonagronomic uses of the present compounds and compositions also include the control of pests such as termites that can damage wood or other structural materials used in buildings.

Nonagronomic uses of the present compounds and compositions also include protecting human and animal health by controlling invertebrate pests that are parasitic or transmit infectious diseases. The controlling of animal parasites includes controlling external parasites that are parasitic to the surface of the body of the host animal (e.g., shoulders, armpits, abdomen, inner part of the thighs) and internal parasites that are parasitic to the inside of the body of the host animal (e.g., stomach, intestine, lung, veins, under the skin, lymphatic tissue). External parasitic or disease transmitting pests include, for example, chiggers, ticks, lice, mosquitoes, flies, mites and fleas. Internal parasites include heartworms, hookworms and helminths. Compounds and compositions of the present invention are particularly suitable for combating external parasitic or disease transmitting pests. Compounds and compositions of the present invention are suitable for systemic and/or non-systemic control of infestation or infection by parasites on animals.

Compounds and compositions of the present invention are suitable for combating parasites that infest animal subjects including those in the wild, livestock and agricultural working animals such as cattle, sheep, goats, horses, pigs, donkeys, camels, bison, buffalos, rabbits, hens, turkeys, ducks, geese and bees (e.g., raised for meat, milk, butter, eggs, fur, leather, feathers and/or wool). By combating parasites, fatalities and performance reduction (in terms of meat, milk, wool, skins, eggs, honey, etc.) are reduced, so that applying a composition comprising a compound of the present invention allows more economic and simple husbandry of animals.

Compounds and compositions of the present invention are especially suitable for combating parasites that infest companion animals and pets (e.g., dogs, cats, pet birds and aquarium fish), research and experimental animals (e.g., hamsters, guinea pigs, rats and mice), as well as animals raised for/in zoos, wild habitats and/or circuses.

In an embodiment of this invention, the animal is preferably a vertebrate, and more preferably a mammal, avian or fish. In a particular embodiment, the animal subject is a mammal (including great apes, such as humans). Other mammalian subjects include primates (e.g., monkeys), bovine (e.g., cattle or dairy cows), porcine (e.g., hogs or pigs), ovine (e.g., goats or sheep), equine (e.g., horses), canine (e.g., dogs), feline (e.g., house cats), camels, deer, donkeys, bison, buffalos, antelopes, rabbits, and rodents (e.g., guinea pigs, squirrels, rats, mice, gerbils, and hamsters). Avians include Anatidae (swrans, ducks and geese), Columbidae (e.g., doves and pigeons), Phasianidae (e.g., partridges, grouse and turkeys), Thesienidae (e.g., domestic chickens), Psittacines (e.g., parakeets, macaws, and parrots), game birds, and ratites (e.g., ostriches).

Birds treated or protected by the inventive compounds can be associated with either commercial or noncommercial aviculture. These include Anatidae, such as swans, geese, and ducks, Columbidae, such as doves and domestic pigeons, Phasianidae, such as partridge, grouse and turkeys, Thesienidae, such as domestic chickens, and Psittacines, such as parakeets, macaws, and parrots raised for the pet or collector market, among others.

For purposes of the present invention, the term “fish” shall be understood to include without limitation, the Teleosti grouping offish, i.e., teleosts. Both the Salmoniformes order (which includes the Salmonidae family) and the Perciformes order (which includes the Centrarchidae family) are contained within the Teleosti grouping. Examples of potential fish recipients include the Salmonidae, Serranidae, Sparidae, Cichlidae, and Centrarchidae, among others.

Other animals are also contemplated to benefit from the inventive methods, including marsupials (such as kangaroos), reptiles (such as farmed turtles), and other economically important domestic animals for which the inventive methods are safe and effective in treating or preventing parasite infection or infestation.

Examples of invertebrate parasitic pests controlled by administering a parasiticidally effective amount of a compound of this invention to an animal to be protected include ectoparasites (arthropods, acarines, etc) and endoparasites (helminths, e.g., nematodes, trematodes, cestodes, acanthocephalans, etc.).

The disease or group of diseases described generally as helminthiasis is due to infection of an animal host with parasitic worms known as helminths. The term ‘helminths’ is meant to include nematodes, trematodes, cestodes and acanthocephalans. Helminthiasis is a prevalent and serious economic problem with domesticated animals such as swine, sheep, horses, cattle, goats, dogs, cats and poultry.

Among the Helminths, the group of worms described as nematodes causes widespread and at times serious infection in various species of animals. Nematodes that are contemplated to be treated by the compounds of this invention and by the inventive methods include, without limitation, the following genera: Acanthocheilonema, Aelurostrongylus, Ancylostoma, Angiostrongylus, Ascaridia, Ascaris, Brugia, Bunostomum, Capillaria, Chabertia, Cooperia, Crenosoma, Dictyocaulus, Dioctophyme, Dipetalonema, Diphyllobothrium, Dirofilaria, Dracunculus, Enterobius, Filaroides, Haemonchus, Heterakis, Lagochilascaris, Loa, Mansonella, Muellerius, Necator, Nematodirus, Oesophagostomum, Osteriagia, Oxyuris, Parafilaria, Parascaris, Physaloptera, Protostrongylus, Setaria, Spirocerca, Stephanofilaria, Strongyloides, Strongylus, Thelazia, Toxascaris, Toxocara, Trichinella, Trichonema, Trichostrongylus, Trichuris, Uncinaria and Wuchereria.

Of the above, the most common genera of nematodes infecting the animals referred to above are Haemonchus, Trichostrongylus, Osteriagia, Nematodirus, Cooperia, Ascaris, Bunostomum, Oesophagostomum, Chabertia, Trichuris, Strongylus, Trichonema, Dictyocaulus, Capillaria, Heterakis, Toxocara, Ascaridia, Oxyuris, Ancylostoma, Uncinaria, Toxascaris and Parascaris. Certain of these, such as Nematodirus, Cooperia and Oesophagostomum attack primarily the intestinal tract while others, such as Haemonchus and Osteriagia, are more prevalent in the stomach while others such as Dictyocaulus are found in the lungs. Still other parasites may be located in other tissues such as the heart and blood vessels, subcutaneous and lymphatic tissue and the like.

Trematodes that are contemplated to be treated, by the compounds of this invention and by the inventive methods include, without limitation, the following genera: Alaria, Fasciola, Nanophyetus, Opisthorchis, Paragonimus and Schistosoma.

Cestodes that are contemplated, to be treated by the compounds of this invention and by the inventive methods include, without limitation, the following genera: Diphyllobothrium, Diplydium, Spirometra and Taenia.

The most common genera of parasites of the gastrointestinal tract of humans are Ancylostoma, Necator, Ascaris, Strongyloides, Trichinella, Capillaria, Trichuris and Enterobius. Other medically important genera of parasites which are found in the blood or other tissues and organs outside the gastrointestinal tract are the filarial worms such as Wuchereria, Brugia, Onchocerca and Loa, as well as Dracunculus and extra intestinal stages of the intestinal worms Strongyloides and Trichinella.

Numerous other Helminth genera and species are known to the art, and are also contemplated to be treated by the compounds of the invention. These are enumerated in great detail in Textbook of Veterinary Clinical Parasitology, Volume 1, Helminths, E. J. L. Soulsby, F. A. Davis Co., Philadelphia, Pa.; Helminths, Arthropods and Protozoa, (6^{th} Edition of Monnig's Veterinary Helminihology and Entomology), E. J. L. Soulsby, The Williams and Wilkins Co., Baltimore, Md.

It is also contemplated that the inventive compounds are effective against a number of ectoparasites of animals, e.g., arthropod ectoparasites of mammals and birds although it is also recognized that some arthropods can be endoparasites as well.

Thus, insect and acarine pests include, e.g., biting insects, such as flies and mosquitoes, mites, ticks, lice, fleas, true bugs, parasitic maggots, and the like.

Adult flies include, e.g., the horn fly or Haematobia irritans, the horse fly or Tabanus spp., the stable fly or Stomoxys calcitrans, the black fly or Simulium spp., the deer fly or Chrysops spp., the louse fly or Melophagus ovinus, the tsetse fly or Glossina spp. Parasitic fly maggots include, e.g., the bot fly (Oestrus ovis and Cuterebra spp.), the blow fly or Phoenicia spp., the screwworm or Cochliomyia hominivorax, the cattle grub or Hypoderma spp., the fleeceworm and the Gastrophiius of horses. Mosquitoes include, for example, Culex spp., Anopheles spp., and Aedes spp.

Mites include Mesostigmata spp. e.g., mesostigmatids such as the chicken mite, Dermanyssus gallinae; itch or scab mites such as Sarcoptidae spp. for example, Sarcoptes scabiei; mange mites such as Psoroptidae spp. including Chorioptes bovis and Psoroptes ovis; chiggers e.g., Trombiculidae spp. for example the North American chigger, Trombicula alfreddugesi.

Ticks include, e.g., soft-bodied ticks including Argasidae spp. for example Argas spp, and Ornithodoros spp.; hard-bodied ticks including Ixodidae spp., for example Rhipicephalus sanguineus, Dermacentor variabilis, Dermacenlor andersoni, Amblyomma americanum, Ixodes scapularis and Boophilus spp.

Lice include, e.g., sucking lice, e.g., Menopon spp. and Bovicola spp.; biting lice, e.g., Haemalopinus spp., Linognathus spp. and Solenopotes spp.

Fleas include, e.g., Ctenocephalides spp., such as dog flea (Ctenocephalides canis) and cat flea (Ctenocephalides felis); Xenopsylla spp. such as oriental rat flea (Xenopsylla cheopis); and Pulex spp. such as human flea (Pulex irritans).

True bugs include, e.g., Cimicidae or e.g., the common bed bug (Cimex lectularius); Triatominae spp. including triatomid bugs also known as kissing bugs; for example Rhodnius prolixus and Triatoma spp.

Generally, flies, fleas, lice, mosquitoes, gnats, mites, ticks and helminths cause tremendous losses to the livestock and companion animal sectors. Arthropod parasites also are a nuisance to humans and can vector disease-causing organisms in humans and animals.

Numerous other arthropod pests and ectoparasites are known to the art, and are also contemplated to be treated by the compounds of the invention. These are enumerated in great detail in Medical and Veterinary Entomology, D. S. Kettle, John Wiley & Sons, New York and Toronto; Control of Arthropod Pests of Livestock: A Review of Technology, R. O. Drummand, J. E, George, and S. E. Kunz, CRC Press, Boca Raton, Fla.

The compounds and compositions of this invention may also be effective against a number of protozoa endoparasites of animals, such as those summarized by Table 1, as follows.

TABLE 1
Exemplary Parasitic Protozoa and Associated Human Diseases

| | | Representative | Human Disease or |
|---|---|---|---|
| Phylum | Subphylum | Genera | Disorder |
| Sarcomastigo- | Mastigophora | Leishmania | Visceral, cutaneous |
| phora (with | (Flagella) | | and mucocutaneous |
| flagella, | | | Infection |
| pseudopodia, | | | |
| or both) | | | |
| | | Trypansoma | Sleeping sickness |
| | | | Chagas' disease |
| | | Giardia | Diarrhea |
| | | Trichomonas | Vaginitis |
| | Sarcodina | Entamoeba | Dysentery, liver |
| | (pseudopodia) | | Abscess |
| | | Dientamoeba | Colitis |
| | | Naegleria and | Central nervous |
| | | Acanthamoeba | system and corneal |
| | | | ulcers |
| | | Babesia | Babesiesis |
| Apicomplexa | | Plasmodium | Malaria |
| (apical | | | |
| complex) | | | |
| | | Isospora | Diarrhea |
| | | Sarcocystis | Diarrhea |
| | | Cryptosporidum | Diarrhea |
| | | Toxoplasma | Toxoplasmosis |
| | | Eimeria | Chicken coccidiosis |
| Microspora | | Enterocytozoon | Diarrhea |
| Ciliaphora | | Balantidium | Dysentery |
| (with cilia) | | | |
| Unclassified | | Pneumocystis | Pneumonia |

In particular, the compounds of this invention are effective against ectoparasites including fleas such as Ctenocephalides felis (cat flea) and Ctenocephalides canis (dog flea).

The compounds of this invention may also be effective against other ectoparasites including flies such as Haematobia (Lyperosia) irritans (horn fly), Stomoxys calcitrans (stable fly), Simulium spp. (blaekfly), Giossina spp. (tsetse flies), Hydrotaea irritans (head fly), Musca autumnalis (face fly), Musca domestica (house fly), Moreliia simplex (sweat fly), Tabanus spp. (horse fly), Hypoderma bovis, Hypoderma lineatum, Lucilia sericata, Lucilia cuprina (green blowfly), Calliphora spp. (blowfly), Protophormia spp., Oestrus ovis (nasal botfly), Culicoides spp. (midges), Hippobosca equine, Gastrophilus instestinalis, Gastrophilus haemorrhoidalis and Gastrophilus naslis; lice such as Bovicola (Damalinia) bovis, Bovicola equi, Haemaiopinus asini, Felicola subrostratus, Heterodoxus spiniger, Lignonathus setosus and Trichodectes canis; keds such as Melophagus ovinus; mites such as Psoroptes spp., Sarcoptes scabei, Chorioptes bovis, Demodex equi, Cheyletiella spp., Notoedres cati, Trombicula spp. and Otodectes cyanotis (ear mites); and ticks such as Ixodes spp., Boophilus spp., Rhipicephalus spp., Amblyomma spp., Dermacentor spp., Hyalomma spp. and Haemaphysalis spp.

Biologically active compounds or agents useful in the compositions of the present invention include the organophosphate pesticides. This class of pesticides has very broad activity as insecticides and, in certain instances, anthelminitic activity. Organophosphate pesticides include, e.g., dicrotophos, terbufos, dimethoate, diazinon, disulfoton, trichlorfon, azinphos-methyl, chlorpyrifos, malathion, oxydemeton-methyl, metharaidophos, acephate, ethyl parathion, methyl parathion, mevinphos, phorate, carbofenthion and phosalone. It is also contemplated to include combinations of the inventive methods and compounds with carbamate type pesticides, including, e.g., carbaryl, carbofuran, aldicarb, molinate, methomyl, carbofuran, etc., as well as combinations with the organochlorine type pesticides. It is further contemplated to include combinations with biological pesticides, including repellents, the pyrethrins (as well as synthetic variations thereof, e.g., allethrin, resmethrin, permethrin, tralomethrin), and nicotine, that is often employed as an acaricide. Other contemplated combinations are with miscellaneous pesticides including: bacillus thuringensis, chlorobenzilate, formamidines (e.g., amitraz), copper compounds (e.g., copper hydroxide and cupric oxychloride sulfate), cyfluthrin, cypermethrin, dicofol, endosulfan, esenfenvalerate, fenvalerate, lambda-cyhalothrin, methoxychlor and sulfur.

Of note are additional biologically active compounds or agents selected from art-known anthelmintics, such as, for example, avermectins (e.g., ivermectin, moxidectin, miibemycin), benzimidazoles (e.g., albendazole, triclabendazole), salicylanilides (e.g., ciosantel, oxyclozanide), substituted phenols (e.g., nitroxynil), pyrimidines (e.g., pyrantel), imidazothiazoles (e.g., ievamisole) and praziquantel.

Other biologically active compounds or agents useful in the compositions of the present invention can be selected from Insect Growth Regulators (IGRs) and Juvenile Hormone Analogues (JHAs) such as diflubenzuron, triflumuron, fiuazuron, cyromazine, methoprene, etc., thereby providing both initial and sustained control of parasites (at all stages of insect development, including eggs) on the animal subject, as well as within the environment of the animal subject.

Of note are biologically active compounds or agents useful in the compositions of the present invention selected from the antiparasitic class of avermectin compounds. As stated above, the avermectin family of compounds is a series of very potent antiparasitic agents known to be useful against a broad spectrum of endoparasites and ectoparasites in mammals.

A notable compound for use within the scope of the present invention is ivermectin. Ivermectin is a semi-synthetic derivative of avermectin and is generally produced as a mixture of at least 80% 22,23-dihydroavermectin B₁ₐ and less than 20% 22,23-dihydroavemiectin B_{1b}. Ivermectin is disclosed in U.S. Pat. No. 4,199,569.

Abamectin is an avermectin that is disclosed as Avermectin B₁ₐ/B_{1b} in U.S. Pat. No. 4,310,519. Abamectin contains at least 80% of avermectin B₁ₐ and not more than 20% of avermectin B_{1b}.

Another notable avermectin is Doramectin, also known as 25-cyclohexyl-avermectin B₁. The structure and preparation of Doramectin is disclosed in U.S. Pat. No. 5,089,480.

Another notable avermectin is Moxidectin. Moxidectin, also known as LL-F28249 alpha, is known from U.S. Pat. No. 4,916,154.

Another notable avermectin is Selamectin. Selamectin is 25-cyclohexyl-25-de(1-methylpropyl)-5-deoxy-22,23-dihydro-5-(hydroxyimino)-avermectin B₁ monosaccharide.

Miibemycin, or B41, is a substance which is isolated from the fermentation broth of a Miibemycin producing strain of Streptomyces. The microorganism, the fermentation conditions and the isolation procedures are more fully described in U.S. Pat. No. 3,950,360 and U.S. Pat. No. 3,984,564.

Emamectin (4″-deoxy-4″-epi-methylaminoavermeetin B₁), which can be prepared as described in U.S. Pat. No. 5,288,710 or U.S. Pat. No. 5,399,717, is a mixture of two homologues, 4″-deoxy-4″-epi-methylaminoavermectin B₁ₐ and 4″-deoxy-4″-epi-methylaminoavermectin B_{1b}. Preferably, a salt of Emamectin is used. Non-limiting examples of salts of Emamectin which can be used in the present invention include the salts described in U.S. Pat. No. 5,288,710, e.g., salts derived from benzoic acid, substituted benzoic acid, benzenesulfonic acid, citric acid, phosphoric acid, tartaric acid, maieic acid, and the like. Most preferably, the Eraamectin salt used in the present invention is Emamectin benzoate.

Eprinomectin is chemically known as 4″-epi-acetylamino-4″-deoxy-avermectin B₁. Eprinoraectin was specifically developed to be used in all cattle classes and age groups. It was the first avermectin to show broad-spectrum activity against both endo- and ecto-parasites while also leaving minimal residues in meat and milk. It has the additional advantage of being highly potent when delivered topically.

The composition of the present invention optionally comprises combinations of one or more of the following antiparasite compounds: imidazo[1,2-b]pyridazine compounds as described by U.S. application Ser. No. 11/019,597, filed on Dec. 22, 2004, and published on Aug. 18, 2005 as US 2005-0182059A1; 1-(4-mono and di-halomethylsulphonylphenyl)-2-acylamino-3-fluoropropanol compounds, as described by U.S. application Ser. No. 11/018,156, filed on Dec. 21, 2004, now U.S. Pat. No. 7,361,689; trifluoromethanesulfonanilide oxime ether derivatives, as described by U.S. application Ser. No. 11/231,423, filed on Sep. 21, 2005, now U.S. Pat. No. 7,312,248; and n-[(phenyloxy)phenyl]-1,1,1-trifluoromethanesulfonamide and n-[(phenylsulfanyl)phenyl]-1,1,1-trifluoromethanesulfonamide derivatives, as described by U.S. Provisional Application Ser. No. 60/688,898, filed on Jun. 9, 2005, and published as US 2006-0281695A1 on Dec. 14, 2006.

The compositions of the present invention can also further comprise a flukicide. Suitable flukicides include, for example, triclabendazole, fenbendazole, albendazole, Clorsulon and oxibendazole. It will be appreciated that the above combinations can further include combinations of antibiotic, antiparasitic and anti-fluke active compounds.

In addition to the above combinations, it is also contemplated to provide combinations of the inventive methods and compounds, as described herein, with other animal health remedies such as trace elements, anti-inflammatories, anti-infectives, hormones, dermatological preparations, including antiseptics and disinfectants, and immunobiologicals such as vaccines and antisera for the prevention of disease.

For example, such antinfectives include one or more antibiotics that are optionally co-administered during treatment using the inventive compounds or methods, e.g., in a combined composition and/or in separate dosage forms. Art-known antibiotics suitable for this purpose include, for example, those listed herein below.

One useful antibiotic is Florfenicol, also known as D-(threo)-1-(4-methylsulfonylphenyl)-2-dichloroacetamido-3-fluoro-1-propanol. Another notable antibiotic compound is D-(threo)-1-(4-methylsulfonyphenyl)-2-difluoroacetamido-3-fluoro-1-propanol. Another useful antibiotic is Thiamphenicol. Processes for the manufacture of these antibiotic compounds, and intermediates useful in such processes, are described in U.S. Pat. No. 4,311,857; U.S. Pat. No. 4,582,918; U.S. Pat. No. 4,973,750; U.S. Pat. No. 4,876,352; U.S. Pat. No. 5,227,494: U.S. Pat. No. 4,743,700; U.S. Pat. No. 5,567,844; U.S. Pat. No. 5,105,009; U.S. Pat. No. 5,382,673; U.S. Pat. No. 5,352,832; and U.S. Pat. No. 5,663,361. Other florfenicol analogs and/or prodrugs have been disclosed and such analogs also can be used in the compositions and methods of the present invention (see e.g., U.S. Patent Application Publication No: 2004/0082553, now U.S. Pat. No. 7,041,670, and U.S. patent application Ser. No. 11/016,794, now U.S. Pat. No. 7,153,842).

Another useful antibiotic compound is Tilmicosin. Tilmicosin is a macrolide antibiotic that is chemically defined as 20-dihydro-20-deoxy-20-(cis-3,5-dimethylpiperidin-1-yl)-desmycosin and which is reportedly disclosed in U.S. Pat. No. 4,820,695.

Another useful antibiotic for use in the present invention is tulathromycin. Tulathromycin is also identified as (2R,3S,4R,5R,8R,10R,11R,12S,13S,14R) 13-[(2,6-dideoxy-3-C-methyl-3-O-methyl-4-C-[(propylamino)methyl]-alpha-L-ribo-hexopyranosyl]-oxy]-2-ethyl-3,4,10-trihydroxy-3,5,8,10,12,14-hexamethyl-11-[[3,4,6-trideoxy-3-(dimethyl-amino)-beta-D-xylo-hexopyranosyl]oxy]-1-oxa-6-azacyclopentadeean-15-one. Tulathromycin can be prepared in accordance with the procedures set forth in U.S. Patent Publication No. 2003/0064939 A1.

Further antibiotics for use in the present invention include the cephalosporins such as, for example, ceftiofur, cefquinome, etc. The concentration of the cephalosporin in the formulation of the present invention optionally varies between about 1 mg/mL to 500 mg/mL.

Another useful antibiotic includes the fluoroquinolones, such as, for example, enrofloxacin, danofloxacin, difioxaein, orbifloxacin and marbofloxacin. Enrofloxacin is typically administered in a concentration of about 100 mg/mL. Danofloxacin is typically administered at a concentration of about 180 mg/mL.

Other useful macrolide antibiotics include compounds from the class of ketolides, or, more specifically, the azalides. Such compounds are described in, for example, U.S. Pat. No. 6,514,945, U.S. Pat. No. 6,472,371, U.S. Pat. No. 6,270,768, U.S. Pat. No. 6,437,151, U.S. Pat. No. 6,271,255, U.S. Pat. No. 6,239,112, U.S. Pat. No. 5,958,888, U.S. Pat. No. 6,339,063 and U.S. Pat. No. 6,054,434.

Other useful antibiotics include the tetracyclines, particularly chlortetracycline and oxytetracycline. Other antibiotics may include β-lactams such as penicillins, e.g., penicillin, ampicillin, amoxicillin, or a combination of amoxicillin with clavulanic acid or other beta lactamase inhibitors.

Nonagronomic applications in the veterinary sector are by conventional means such as by enteral administration in the form of, for example, tablets, capsules, drinks, drenching preparations, granulates, pastes, boli, feed-through procedures, or suppositories; or by parenteral administration, such as by injection (including intramuscular, subcutaneous, intravenous, intraperitoneal) or implants; by nasal administration; by topical administration, for example, in the form of immersion or dipping, spraying, washing, coating with powder, or application to a small area of the animal, and through articles such as neck collars, ear tags, tail bands, limb bands or halters which comprise compounds or compositions of the present invention.

Any of the compounds of the present invention, or a suitable combination of such compounds, may be administered directly to the animal subject and/or indirectly by applying it to the local environment in which the animal dwells (such as bedding, enclosures, or the like). Direct administration includes contacting the skin, fur or feathers of a subject animal with the compounds, or by feeding or injecting the compounds into the animal

The compounds of the present invention may be administered in a controlled release form, e.g., in a subcutaneous slow release formulation, or in the form of a controlled, release device affixed to an animal such as a fleacollar. Collars for the controlled release of an insecticide agent for long term protection against flea infestation in a companion animal are art-known, and are described, for example, by U.S. Pat. No. 3,852,416, U.S. Pat. No. 4,224,901, U.S. Pat. No. 5,555,848 and U.S. Pat. No. 5,184,573.

Typically a parasiticidal composition according to the present invention comprises a mixture of a compound of Formula 1, an N-oxide or salt thereof, with one or more pharmaceutically or veterinarily acceptable carriers comprising excipients and auxiliaries selected, with regard to the intended route of administration (e.g., oral, topical or parenteral administration such as injection) and in accordance with standard practice. In addition, a suitable carrier is selected on the basis of compatibility with the one or more active ingredients in the composition, including such considerations as stability relative to pH and moisture content. Therefore of note is a composition for protecting an animal from an invertebrate parasitic pest comprising a parasitically effective amount of a compound of the invention and at least one carrier.

For parenteral administration including intravenous, intramuscular and subcutaneous injection, a compound of the present invention can be formulated, in suspension, solution or emulsion in oily or aqueous vehicles, and may contain adjuncts such as suspending, stabilizing and/or dispersing agents. The compounds of the present invention may also be formulated for bolus injection or continuous infusion. Pharmaceutical compositions for injection include aqueous solutions of water-soluble forms of active ingredients (e.g., a salt of an active compound), preferably in physiologically compatible buffers containing other excipients or auxiliaries as are known in the art of pharmaceutical formulation. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Formulations for injection may be presented in unit dosage form, e.g., in ampoules or in multi-dose containers. Alternatively, the active ingredient may be in powder form for constitution with a suitable vehicle, e.g., sterile, pyrogen-free water, before use.

In addition to the formulations described supra, the compounds of the present invention may also be formulated as a depot preparation. Such long acting formulations may be administered by implantation (for example, subcutaneously or intramuscularly) or by intramuscular or subcutaneous injection. The compounds of the present invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

For administration by inhalation, the compounds of the present invention can be delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

Compounds of the present invention have been discovered to have favorable pharmacokinetic and pharmacodynamic properties providing systemic availability from oral administration and ingestion. Therefore after ingestion by the animal to be protected, parasiticidally effective concentrations of compounds of the invention in the bloodstream protect the treated animal from blood-sucking pests such as fleas, ticks and lice. Therefore of note is a composition for protecting an animal from an invertebrate parasite pest in a form for oral administration (i.e. comprising, in addition to a parasiticidally effective amount of a compound of the invention, one or more carriers selected, from binders and fillers suitable for oral administration and feed concentrate carriers).

For oral administration in the form of solutions (the most readily available form for absorption), emulsions, suspensions, pastes, gels, capsules, tablets, boluses, powders, granules, rumen-retention and feed/water/lick blocks, a compound of the present invention can be formulated with binders/fillers known in the art to be suitable for oral administration compositions, such as sugars and sugar derivatives (e.g., lactose, sucrose, mannitol, sorbitol), starch (e.g., maize starch, wheat starch, rice starch, potato starch), cellulose and derivatives (e.g., methylcellulose, carboxymethylcellulose, ethylhydroxycellulose), protein derivatives (e.g., zein, gelatin), and synthetic polymers (e.g., polyvinyl alcohol, polyvinylpyrrolidone). If desired, lubricants (e.g., magnesium stearate), disintegrating agents (e.g., cross-linked polyvinylpyrrolidinone, agar, alginic acid) and dyes or pigments can be added. Pastes and gels often also contain adhesives (e.g., acacia, alginic acid, bentonite, cellulose, xanthan gum, colloidal magnesium aluminum silicate) to aid in keeping the composition in contact with the oral cavity and not being easily ejected.

If the parasiticidal compositions are in the form of feed concentrates, the carrier is typically selected from high-performance feed, feed cereals or protein concentrates. Such feed concentrate-containing compositions can, in addition to the parasiticidal active ingredients, comprise additives promoting animal health or growth, improving quality of meat from animals for slaughter or otherwise useful to animal husbandry. These additives can include, for example, vitamins, antibiotics, chemotherapeutics, bacteriostats, fungistats, coccidiostats and hormones.

The compounds of Formula 1 may also be formulated in rectal compositions such as suppositories or retention enemas, using, e.g., conventional suppository bases such as cocoa butter or other glycerides.

Formulations for topical administration are typically in the form of a powder, cream, suspension, spray, emulsion, foam, paste, aerosol, ointment, salve or gel. More typically a topical formulation is a water-soluble solution, which can be in the form of a concentrate that is diluted before use. Parasiticidal compositions suitable for topical administration typically comprise a compound, of the present invention and one or more topically suitable carriers. In applications of a parasiticidal composition topically to the exterior of an animal as a line or spot (i.e. “spot-on” treatment), the active ingredient migrates over the surface of the animal to cover most or all of its external surface area. As a result, the treated animal is particularly protected from invertebrate pests that feed off the epidermis of the animal such as ticks, fleas and lice. Therefore formulations for topical localized administration often comprise at least one organic solvent to facilitate transport of the active ingredient over the skin and/or penetration into the epidermis of the animal. Carriers in such formulations include propylene glycol, paraffins, aromatics, esters such as isopropyl myristate, glycol ethers, alcohols such as ethanol, n-propanol, 2-octyl dodecanol or oleyi alcohol; solutions in esters of monocarboxylic acids, such as isopropyl myristate, isopropyl palmitate, lauric acid, oxalic ester, oleic acid oleyi ester, oleic acid decyl ester, hexyl laurate, oieyl oleate, decyl oleate, caproic acid esters of saturated fatty alcohols of chain length C₁₂-C₁₈; solutions of esters of dicarboxylic acids, such as dibutyl phthalate, diisopropyl isophthalate, adipic acid diisopropyl ester, di-n-butyl adipate or solutions of esters of aliphatic acids, e.g., glycols. It may be advantageous for a crystallization inhibitor or a dispersant known from the pharmaceutical or cosmetic industry also to be present.

A pour-on formulation may also be prepared for control of parasites in an animal of agricultural worth. The pour-on formulations of this invention can be in the form of a liquid, powder, emulsion, foam, paste, aerosol, ointment, salve or gel. Typically, the pour-on formulation is liquid. These pour-on formulations can be effectively applied to sheep, cattle, goats, other ruminants, camelids, pigs and horses. The pour-on formulation is typically applied by pouring in one or several lines or in a spot-on the dorsal midline (back) or shoulder of an animal. More typically, the formulation is applied by pouring it along the back of the animal, following the spine. The formulation can also be applied to the animal by other conventional methods, including wiping an impregnated material over at least a small area of the animal, or applying it using a commercially available applicator, by means of a syringe, by spraying or by using a spray race. The pour-on formulations include a carrier and can also include one or more additional ingredients. Examples of suitable additional ingredients are stabilizers such as antioxidants, spreading agents, preservatives, adhesion promoters, active solubilisers such as oleic acid, viscosity modifiers, UV blockers or absorbers, and colourants. Surface active agents, including anionic, cationic, non-ionic and ampholytic surface active agents, can also be included in these formulations.

The formulations of this invention typically include an antioxidant, such as BHT (butylated hydroxy toluene). The antioxidant is generally present in amounts of at 0.1-5% (wt/vol). Some of the formulations require a solubilizer, such as oleic acid, to dissolve the active agent, particularly if spinosad is used. Common spreading agents used in these pour-on formulations are: IPM, IPP, caprylic/capric acid esters of saturated C₁₂-C₁₈ fatty alcohols, oleic acid, oleyl ester, ethyl oleate, triglycerides, silicone oils and DPM. The pour-on formulations of this invention are prepared according to known techniques. Where the pour-on is a solution, the parasiticide/insecticide is mixed with the carrier or vehicle, using heat and stirring where required. Auxiliary or additional ingredients can be added to the mixture of active agent and carrier, or they can be mixed with the active agent prior to the addition of the carrier. If the pour-on is an emulsion or suspension, these formulations are similarly prepared, using known techniques.

Other delivery systems for relatively hydrophobic pharmaceutical compounds may be employed. Liposomes and emulsions are well-known examples of delivery vehicles or carriers for hydrophobic drugs. In addition, organic solvents such as dimethylsulfoxide may be used, if needed.

For agronomic applications, the rate of application required for effective control (i.e. “biologically effective amount”) will depend, on such factors as the species of invertebrate to be controlled, the pest's life cycle, life stage, its size, location, time of year, host crop or animal, feeding behavior, mating behavior, ambient moisture, temperature, and the like. Under normal circumstances, application rates of about 0.01 to 2 kg of active ingredients per hectare are sufficient to control pests in agronomic ecosystems, but as little as 0.0001 kg/hectare may be sufficient or as much as 8 kg/hectare may be required. For nonagronomic applications, effective use rates will range from about 1.0 to 50 mg/square meter but as little as 0.1 mg/square meter may be sufficient or as much as 150 mg/square meter may be required. One skilled in the art can easily determine the biologically effective amount necessary for the desired level of invertebrate pest control.

In general for veterinary use, a compound of Formula 1, an N-oxide or salt thereof, is administered in a parasiticidally effective amount to an animal to be protected from invertebrate parasite pests. A parasiticidally effective amount is the amount of active ingredient needed to achieve an observable effect diminishing the occurrence or activity of the target invertebrate parasite pest. One skilled in the art will appreciate that the parasitically effective dose can vary for the various compounds and compositions of the present invention, the desired parasitical effect and duration, the target invertebrate pest species, the animal to be protected, the mode of application and the like, and the amount needed to achieve a particular result can be determined through simple experimentation.

For oral administration to homeothermic animals, the daily dosage of a compound of the present invention typically ranges from about 0.01 mg/kg to about 100 mg/kg, more typically from about 0.5 mg/kg to about 100 mg/kg, of animal body weight. For topical (e.g., dermal) administration, dips and sprays typically contain from about 0.5 ppm to about 5000 ppm, more typically from about 1 ppm to about 3000 ppm, of a compound of the present invention.

Representative compounds of this invention prepared by the methods described, herein are shown in Index Table A. For mass spectral data (AP⁺ (M+1)), the numerical value reported is the molecular weight of the parent molecular ion (M) formed by addition of H+ (molecular weight of 1) to the molecule to give a M+1 peak observed by mass spectrometry using atmospheric pressure chemical ionization (AP⁺). The alternate molecular ion peaks (e.g., M+2 or M+4) that occur with compounds containing multiple halogens are not reported.

The following additional abbreviations are used in Index Table A: Cmpd means Compound, i-Pr is isopropyl, c-Pr is cyclopropyl, Ph is phenyl, and a “—” entry in the column headed “Z” represents a direct bond.

Fragments A-1 through A-11 and B-1 through B-18 shown below are referred to in Index Table A. The asterisk * denotes the attachment point of the fragment to the remainder of the molecule.

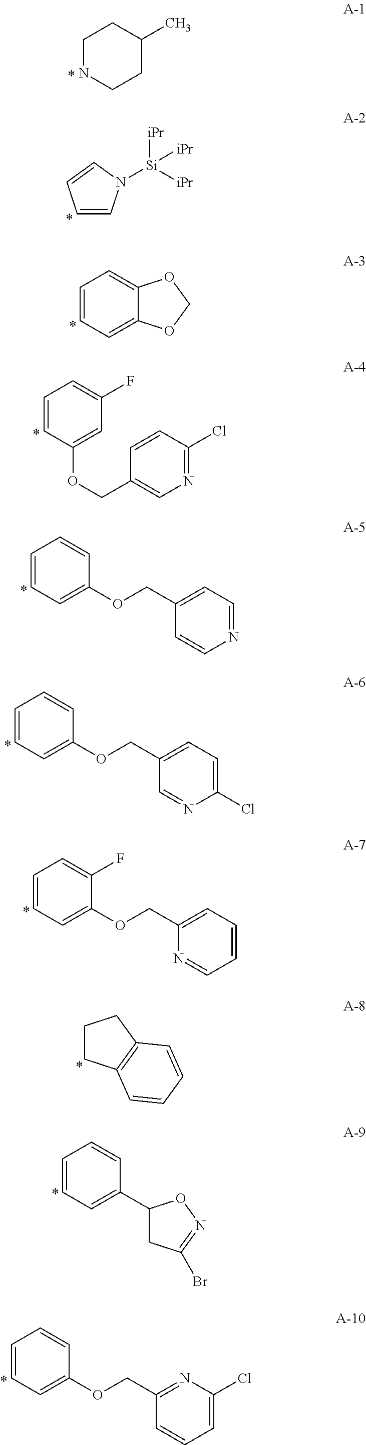

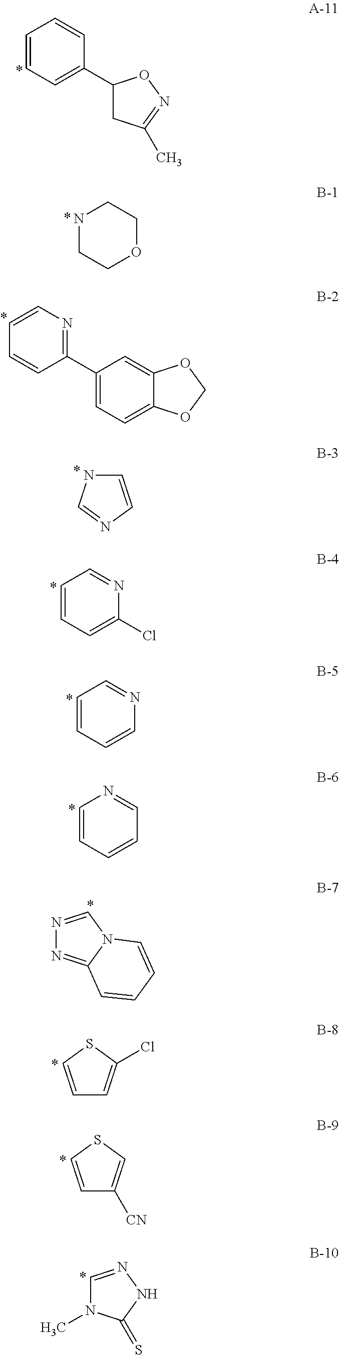

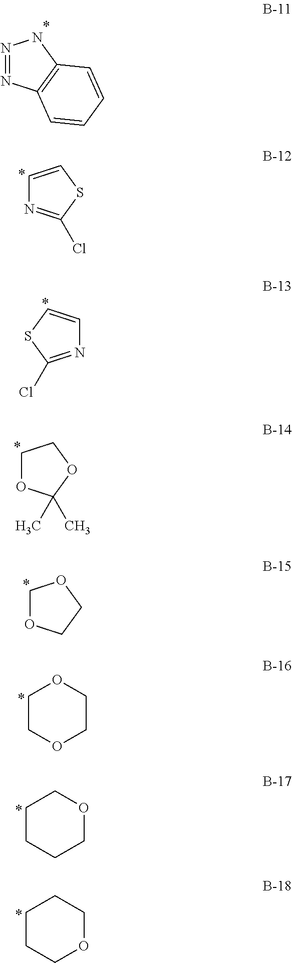

INDEX TABLE A

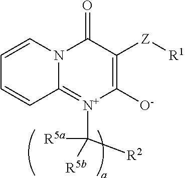

| | | | | | | | AP+ |
|---|---|---|---|---|---|---|---|
| INcode | Cmpd | Z | R¹ | (CR^{5a}R^{5b})ₐ | R² | m.p. (° C.) | (M + 1) |
| QCG51 | 1 | CH₂ | phenyl | CH₂ | CH₂CH₃ | | 295 |
| QCM37 | 2 | — | phenyl | CH₂ | phenyl | | 329 |
| QCN25 | 3 | — | phenyl | CH₂ | H | | 253 |
| QDZ23 | 4 | O | phenyl | CH₂ | CH₂CH₃ | ** | 297 |
| QEB97 | 5 | — | phenyl | CH₂ | CH₂CO₂CH₃ | | 325 |
| QEC92 | 6 | NH | phenyl | CH₂ | CH₂CH₃ | # | |
| QEP31 | 7 | O | 3-(trifluoromethyl)phenyl | CH₂ | CH₂CH₃ | | 365 |
| QEP32 | 8 | O | 4-(trifluoromethyl)phenyl | CH₂ | CH₂CH₃ | # | |
| QEP33 | 9 | O | 3,5-difluorophenyl | CH₂ | CH₂CH₃ | # | |
| QEP34 | 10 | O | 2,4-difluorophenyl | CH₂ | CH₂CH₃ | | 333 |
| QEQ21 | 11 | NH | 3-(trifluoromethyl)phenyl | CH₂ | CH₂CH₃ | # | |
| QEQ22 | 12 | NH | 4-(trifluoromethyl)phenyl | CH₂ | CH₂CH₃ | | 364 |
| QEQ23 | 13 | NH | 4-cyanophenyl | CH₂ | CH₂CH₃ | | 321 |
| QEQ24 | 14 | NH | 2-(trifluoromethyl)phenyl | CH₂ | CH₂CH₃ | # | |
| QER79 | 15 | — | A-1 | CH₂ | CH₂CH₃ | # | |
| QER80 | 16 | NH | 2,4-difluorophenyl | CH₂ | CH₂CH₃ | # | |
| QER81 | 17 | NH | 3,5-difluorophenyl | CH₂ | CH₂CH₃ | # | |
| QES88 | 18 | — | phenyl | CHCH₃ | CO₂CH₂CH₃ | | 339 |
| QEY48 | 19 | — | A-2 | CH₂ | CH₂CH₃ | 163-164 | |
| QKE23 | 20 | — | phenyl | CH₂ | 4-chlorophenyl | | 363 |
| QKE24 | 21 | — | 3-(trifluoromethoxy)phenyl | CH₂ | 4-chlorophenyl | | 447 |
| QKE25 | 22 | — | 4-fluorophenyl | CH₂ | 4-chlorophenyl | | 381 |
| QKQ76 | 23 | — | phenyl | CH₂CH₂ | N(CH₃)₂ | | 310 |
| QKQ77 | 24 | — | 4-fluorophenyl | CH₂CH₂ | N(CH₃)₂ | | 328 |
| QKS40 | 25 | — | 2,4-difluorophenyl | CH₂CH₂ | N(CH₃)₂ | | 346 |
| QKS41 | 26 | — | phenyl | CH₂CH₂CH₂ | B-1 | | 366 |
| QKS42 | 27 | — | 2,4-difluorophenyl | CH₂CH₂CH₂ | B-1 | | 402 |
| QKU13 | 28 | — | phenyl | CH₂CH₂ | B-1 | 183-185 | |
| QKU14 | 29 | — | 2,4-difluorophenyl | CH₂CH₂ | B-1 | 179-181 | |
| QKU18 | 30 | — | 4-fluorophenyl | CH₂ | C(O)NHCH₂CF₃ | | 396 |
| QKU19 | 31 | — | 3-(trifluoromethoxy)phenyl | CH₂ | C(O)NHCH₂CF₃ | | 462 |
| QKU37 | 32 | — | phenyl | CH₂ | C(O)NHCH₂CF₃ | | 378 |
| QKU72 | 33 | — | A-3 | CH₂ | B-2 | | 494 |
| QKU73 | 34 | — | phenyl | CH₂CH₂CH₂ | B-3 | | 347 |
| QKU74 | 35 | — | phenyl | CH₂CH₂ | NHC(O)CH₃ | 175-177 | |
| QKU75 | 36 | — | 2,4-difluorophenyl | CH₂CH₂ | NHC(O)CH₃ | 187-188 | |
| QKW11 | 37 | — | 3-(OCH₂CN)phenyl | CH₂ | CF₃ | 195-196 | ** |
| QKW14 | 38 | — | 4-fluorophenyl | CH₂CH₂ | B-1 | 203-204 | |
| QKW15 | 39 | — | 4-fluorophenyl | CH₂CH₂ | NHC(O)CH₃ | 198-200 | |
| QKW40 | 40 | — | 2,4-difluorophenyl | CH₂ | C(O)NHCH₂CF₃ | | 414 |
| QKY91 | 41 | — | 4-fluorophenyl | CH₂CH₂CH₂ | B-1 | 104-105 | |
| QLA16 | 42 | — | 3-(C(O)NHCH₂CF₃)phenyl | CH₂ | B-4 | | 489 |
| QLD87 | 43 | — | 3-(OCH₂CN)phenyl | CH₂ | B-4 | 189-191 | |
| QLE93 | 44 | — | CN | CH₂ | CF₃ | 229-230 | |
| QLG06 | 45 | — | CH₂CH₂CN | CH₂ | CF₃ | | 298 |
| QLK89 | 46 | — | A-4 | CH₂ | CF₃ | | 480 |
| QLM32 | 47 | — | 4-fluorophenyl | CH₂CH₂ | B-5 | # | |
| QLM36 | 48 | — | phenyl | CH₂CH₂ | B-5 | | 344 |
| QLM39 | 49 | — | A-5 | CH₂ | CF₃ | 223-225 | |
| QLM40 | 50 | — | A-6 | CH₂ | CF₃ | | 462 |
| QLM41 | 51 | — | A-6 | CH₂ | B-4 | | 505 |
| QLQ03 | 52 | — | 3-(trifluoromethoxy)phenyl | CH₂CH₂ | B-5 | | 428 |
| QLQ05 | 53 | — | 4-fluorophenyl | CH₂CH₂ | B-6 | # | |
| QLT24 | 54 | — | phenyl | CH₂CH₂ | B-6 | # | |
| QLW59 | 55 | — | 3-(OCH₂CF₃)phenyl | CH₂ | B-4 | # | |
| QNS52 | 56 | — | A-7 | CH₂ | CF₃ | | 446 |
| QNS51 | 57 | — | A-7 | CH₂ | B-4 | | 489 |
| QNZ08 | 58 | — | 4-fluorophenyl | CH(c-Pr) | B-4 | # | |
| QNZ09 | 59 | — | A-8 | CH₂ | B-4 | | 404 |
| QPB41 | 60 | — | phenyl | CH₂ | 3,4-dichlorophenyl | | 397 |
| QPB42 | 61 | — | phenyl | CH₂ | 3-chlorophenyl | # | |
| QPB47 | 62 | — | phenyl | CHCF₃ | B-4 | # | |
| QPD86 | 63 | — | 4-fluorophenyl | CHCF₃ | B-4 | | 450 |
| QPE68 | 64 | — | A-10 | CH₂ | CF₃ | 170-172 | |
| QPF09 | 65 | — | phenyl | CH₂ | 3-cyanophenyl | # | |
| QPF10 | 66 | — | phenyl | CH₂ | 4-cyanophenyl | | 354 |
| QPF11 | 67 | — | phenyl | CH₂ | 3-chloro-4-n-propylthiophenyl | | 437 |
| QPF31 | 68 | — | phenyl | CH₂ | B-7 | # | |
| QPG15 | 69 | — | phenyl | CH₂ | B-8 | # | |
| QPG16 | 70 | — | phenyl | CH₂ | B-9 | | 360 |
| QPG37 | 71 | — | 2,4-difluorophenyl | CHCF₃ | B-4 | | 468 |
| QPG38 | 72 | — | phenyl | CH(c-Pr) | B-4 | | 404 |
| QPG39 | 73 | — | 2,4-difluorophenyl | CH(c-Pr) | B-4 | | 440 |
| Q7Q20 | 74 | — | phenyl | CH₂ | B-10 | | 366 |
| Q8X71 | 75 | — | phenyl | CH₂CH₂ | CH(OCH₃)₂ | | 341 |
| Q8X72 | 76 | — | phenyl | CH₂ | CH(OCH₃)₂ | | 327 |
| RAB75 | 77 | — | phenyl | CH₂ | B-11 | | 370 |
| RAC76 | 78 | — | CH₂CH₂Ph | CH₂ | B-12 | | 398 |
| RAC77 | 79 | — | CH₂CH₂Ph | CH₂ | B-4 | | 392 |
| RBQ76 | 80 | — | CH₂CH₂Ph | CH₂ | B-13 | | 398 |
| RCV59 | 81 | — | phenyl | CH₂ | 3-methoxyphenyl | | 359 |
| RCV60 | 82 | — | phenyl | CH₂ | 4-methoxyphenyl | | 359 |
| RCX16 | 83 | — | phenyl | CH₂ | 2-methoxyphenyl | | 359 |
| RCZ42 | 84 | — | phenyl | CH₂ | SCH₂CF₃ | | 367 |
| RDF07 | 85 | — | A-8 | CH₂ | B-13 | | 410 |
| RDF42 | 86 | — | phenyl | CHCH₃ | SCH₂CF₃ | | 381 |
| RDH02 | 87 | — | A-9 | CH₂ | B-13 | | 517 |
| RDH03 | 88 | — | A-11 | CH₂ | B-13 | ** | 453 |
| RDH54 | 89 | — | phenyl | CH₂ | B-14 | | 353 |
| RDL66 | 90 | — | phenyl | CH₂ | B-15 | | 325 |
| RDL67 | 91 | — | phenyl | CH₂ | B-16 | | 339 |
| Q9Y73 | 92 | | 3-(C(CH₃)═NOCH₃)phenyl | CH₂ | B-13 | # | |
| RDS74 | 93 | — | phenyl | CH₂ | B-17 | | 337 |
| RDS75 | 94 | — | phenyl | CH₂ | B-18 | | 337 |
| REE65 | 95 | S | CCl₃ | CH₂ | B-13 | # | |
| REG84 | 96 | S | 2-fluorophenyl | CH₂ | B-13 | | 420 |
| REG85 | 97 | S | 2-fluorophenyl | CH₂ | B-4 | | 414 |
| REJ20 | 98 | S(O) | 2-fluorophenyl | CH₂ | B-13 | | 436 |
| REJ19 | 99 | S(O)₂ | 2-fluorophenyl | CH₂ | B-13 | # | |
| REK17 | 100 | S | C(O)OCH₃ | CH₂ | B-4 | | 378 |
| REK18 | 101 | S | C(O)OCH₃ | CH₂ | B-13 | | 384 |
| REK65 | 102 | S | CF₃ | CH₂ | B-13 | # | |
| REL22 | 103 | S | 2-methoxyphenyl | CH₂ | B-4 | | 426 |
| REL23 | 104 | S | 2-methoxyphenyl | CH₂ | B-13 | | 432 |
| REL24 | 105 | S | CF₃ | CH₂ | B-4 | # | |
| REL25 | 106 | S | CF₃ | CH₂ | CF₃ | # | |
| REN78 | 107 | — | 3-(CH₂CN)phenyl | CH₂ | B-13 | | 409 |
| REN79 | 108 | — | C(O)NHCH₂CF₃ | CH₂ | B-13 | | 495 |
| REP05 | 109 | S(O) | CF₃ | CH₂ | CF₃ | | 361 |
| REP06 | 110 | S(O)₂ | CF₃ | CH₂ | CF₃ | | 377 |
| RFT89 | 111 | S(O)₂ | CF₃ | CH₂ | B-13 | # | |
| RFW13 | 112 | S(O)₂ | CF₃ | CH₂ | B-4 | # | |
| RFY56 | 113 | — | 4-(CH₂CN)phenyl | CH₂ | B-13 | | 409 |
| RKY69 | 114 | — | 2-amino-5-(CF₃)phenyl | CH₂ | B-13 | # | |
| RKY70 | 115 | — | 3-aminophenyl | CH₂ | B-13 | # | |
| RLB46 | 116 | — | 3-(triethylsilyl)phenyl | CH₂ | B-13 | | 484 |
| RLC87 | 117 | — | 3-(OSi(t-Bu)Me₂)phenyl | CH₂ | B-13 | | 500 |
| RLD48 | 118 | — | 3-(triisopropysilyloxy)phenyl | CH₂ | B-13 | | 542 |
| RNN08 | 119 | — | 3-(OC(O)OEt)phenyl | CH₂ | B-13 | | 458 |
| RNX37 | 120 | — | CH₂CH₂CN | CH₂ | B-13 | | 347 |
| RNZ32 | 121 | — | CH₂CH═CHPh | CH₂ | B-13 | | 410 |
| RQW19 | 122 | — | C(O)CH₂CH₂Ph | CH₂ | B-13 | | 426 |
| RRT24 | 123 | — | C(O)CH₂(2,6-difluorophenyl) | CH₂ | B-13 | | 448 |
| RRW69 | 124 | — | phenyl | CH₂ | C(O)N(CH₃)₂ | 241.4-272.6 | |
| RSE93 | 125 | — | 2-fluorophenyl | CH₂ | C(O)N(CH₃)₂ | 242.5-243.8 | |
| RXP41 | 126 | — | 3-(trifluoromethoxy)phenyl | CH₂ | C(O)N(CH₃)₂ | | 408 |

# See Index Table B for ¹H NMR data.
** See synthesis example for ¹H NMR data.

INDEX TABLE B

| Cmpd No. | ¹H NMR Data^{a, b} |
|---|---|
| 6 | δ 10.27 (s, 1H), 9.91 (d, 1H), 7.69 (d, 2H), 7.50 (t, 1H), 7.33 (t, 2H), 7.18 (m, 3H), 7.05 (t, |
| | 1H), 4.05 (t, 2H), 1.86 (m, 2H), 1.04 (t, 3H). |
| 8 | δ 9.57 (d, 1H), 7.66 (d, 2H), 7.60 (t, 1H), 7.41 (d, 2H), 7.17 (m, 2H), 4.02 (t, 2H), 1.84 (m, |
| | 2H), 1.03 (t, 3H). |
| 9 | δ 9.55 (d, 1H), 7.61 (t, 2H), 7.17 (t, 2H), 6.87 (m, 2H), 6.69 (m, 1H), 4.01 (t, 2H), 1.84 (m, |
| | 2H), 1.03 (t, 3H). |
| 11 | δ 10.47 (s, 1H), 9.87 (d, 1H), 8.10 (s, 1H), 7.79 (d, 1H), 7.54 (t, 1H), 7.42 (t, 1H), 7.28 (d, |
| | 1H), 7.19 (m. 2H), 4.05 (t, 2H), 1.86 (m, 2H), 1.04 (t, 3H). |
| 14 | δ 10.53 (s, 1H), 9.86 (d, 1H), 8.38 (d, 1H), 7.63 (d, 1H), 7.52 (m, 2H), 7.19 (m. 3H), 4.08 (t, |
| | 2H), 1.85 (m, 2H), 1.03 (t, 3H). |
| 15 | δ 9.21 (d, 1H), 7.37 (t, 1H), 7.07 (d, 1H), 7.00 (t, 1H), 4.3 (br s, 2H), 2.99 (br s, 2H), 1.78 (m, |
| | 4H), 1.3 (m, 1H), 0.98 (d, 3H). |
| 16 | δ 10.42 (s, 1H), 9.84 (d, 1H), 8.42 (m, 1H), 7.53 (t, 1H), 7.20 (m, 2H), 6.88 (m, 2H), 4.05 (t, |
| | 2H), 1.86 (m, 2H), 1.03 (t, 3H). |
| 17 | δ 10.43 (s, 1H), 9.84 (d, 1H), 7.75 (t, 1H), 7.31 (m, 2H), 7.20 (m, 2H), 4.04 (t, 2H), 1.85 (m, |
| | 2H), 1.03 (t, 3H). |
| 47 | δ 9.53 (d, 1H), 8.57 (s, 1H), 8.53 (s, 1H), 8.07 (t, 1H), 7.74 (m, 2H), 7.67 (d, 1H), 7.39 (d, |
| | 1H), 7.35 (t, 1H), 4.55 (t, 2H), 3.11 (t, 2H). |
| 53 | δ 9.51 (d, 1H), 8.59 (d, 1H), 8.07 (t, 1H), 7.87 (d, 1H), 7.76 (m, 2H), 7.62 (t, 1H), 7.32 (t, |
| | 1H), 7.18 (m, 1H), 7.09 (t, 2H), 4.72 (dd, 2H), 3.25 (dd, 2H). |
| 54 | δ 9.51 (d, 1H), 8.59 (d, 1H), 8.00 (t, 1H), 7.85 (d, 1H), 7.77 (d, 2H), 7.62 (t, 1H), 7.40 (t, |
| | 2H), 7.17-7.30 (m, 4H), 4.71 (dd, 2H), 3.25 (dd, 2H). |
| 55 | δ 9.58 (dd, 1H), 8.48 (s, 1H), 8.07 (dd, 1H), 7.70 (m, 1H), 7.51 (d, 1H), 7.43-7.31 (m, 5H), |
| | 6.85 (dd, 1H), 5.65-5.55 (m, 2H), 4.38 (q, 2H). |
| 58 | δ 9.57 (d, 1H), 8.70 (s, 1H), 7.87 (t, 1H), 7.79 (m, 2H), 7.60 (d, 1H), 7.33 (d, 2H), |
| | 7.25 (overlapping CHCl₃, 1H), 7.11 (t, 3H), 6.44 (m, 1H), 1.73 (m, 1H), 1.31 (m, 2H), 0.75 (m, 2H). |
| 61 | δ (acetone-d₆) 9.45 (d, 1H), 8.25 (t, 1H), 7.90 (d, 2H), 7.75 (d, 1H), 7.65 (t, 1H), 7.50 (s, |
| | 1H), 7.39 (m, 2H), 7.35-7.25 (m, 4H), 7.15 (t, 1H), 5.70 (bs, 2H). |
| 62 | δ 9.65 (d, 1H), 8.5 (s, 1H), 8.28 (m, 1H), 7.93 (m, 1H), 7.78 (d, 2H), 7.67 (d, 1H), 7.43 (m, |
| | 3H), 7.15-7.30 (m, 2H). |
| 65 | δ 9.58 (d, 1H), 8.15 (m, 1H), 7.86 (d, 2H), 7.63 (m, 3H), 7.50 (m, 1H), 7.42-7.35 (m, 5H), |
| | 7.23 (m, 1H), 5.60 (bs, 2H). |
| 68 | δ 9.51 (d, 1H), 9.04 (d, 1H), 8.92 (d, 1H), 8.24 (t, 1H), 7.73 (m, 3H), 7.44 (m, 3H), |
| | 7.2-7.40 (m, 2H), 6.92 (t, 1H), 6.2 (br s, 2H). |
| 69 | δ (DMSO-d₆) 9.35 (d, 1H), 8.39 (t, 1H), 8.15 (d, 1H), 7.73 (d, 2H), 7.55 (t, 1H), |
| | 7.35-7.25 (m, 4H), 7.19 (m, 1H), 6.99 (m, 1H), 5.60 (bs, 2H). |
| 92 | δ 9.52 (d, 1H), 8.10 (t, 1H), 8.03 (s, 1H), 7585 (d, 1H), 7.65 (s, 1H), 7.54 (m, 2H), 7.38 (dd, |
| | 2H), 5.56 (br s, 2H), 3.98 (s, 3H), 2.24 (s, 3H). |
| 95 | δ 9.22 (d, 1H), 8.42 (t, 1H), 8.16 (d, 1H), 7.99 (s, 1H), 7.51 (t, 1H), 5.61 (s, 2H). |
| 99 | δ (acetone-d₆) 9.29 (d, 1H), 8.52 (t, 1H), 8.19-8.09 (m, 2H), 7.86 (s, 1H), 7.70-7.62 (m, 2H), |
| | 7.38 (t, 1H), 7.19 (t, 1H), 5.59 (s, 2H). |
| 102 | δ 9.22 (d, 1H), 8.42 (t, 1H), 8.16 (d, 1H), 7.96 (s, 1H), 7.58 (t, 1H), 5.59 (s, 2H). |
| 105 | δ 9.23 (d, 1H), 8.44 (d, 1H), 8.32 (dt, 1H), 7.76 (apparent d, 2H), 7.53 (t, 1H), 5.54 (s, 2H). |
| 106 | δ 9.27 (dd, 1H), 8.49 (dt, 1H), 8.12 (d, 1H), 7.63 (t, 1H), 5.30 (q, 2H). |
| 111 | δ 9.30 (dd, 1H), 8.59 (dt, 1H), 8.22 (d, 1H), 7.92 (s, 1H), 7.70 (t, 1H), 5.69 (s, 2H). |
| 112 | δ (acetone-d₆) 9.31 (d, 1H), 8.58-8.48 (m, 2H), 7.90-7.83 (m, 2H), 7.65 (t, 1H), 7.42 (d, 1H), |
| | 5.63 (d, 2H). |
| 114 | δ (DMSO-d₆) 9.28 (d, 1H), 8.58 (s, 1H), 8.40 (t, 1H), 8.17 (d, 1H), 7.99 (s, 2H), |
| | 7.44-7.58 (m, 3H), 7.27 (d, 1H), 5.63 (d, 2H). |
| 115 | δ 9.55 (d, 1H), 8.14 (t, 1H), 7.66 (s, 1H), 7.58 (d, 1H), 7.40 (d, 1H), 7.12-7.21 (m, 4H), |
| | 7.06 (s, 1H), 6.64 (d, 1H) 5.58 (s, 2H). |

^{a}¹H NMR data are in ppm downfield from tetramethylsilane. CDCl₃ solution unless indicated otherwise; “acetone-d₆” is CD₃C(═O)CD₃ and “DMSO-d₆” is CD₃S(═O)CD₃. Couplings are designated by (s)—singlet, (d)—doublet, (t)—triplet, (m)—multiplet, (dd)—doublet of doublets, (ddd)—doublet of doublet of doublets, (dt)—doublet of triplets, (td)—triplet of doublets, (br)—broad.
^{b}¹H NMR spectra of compounds wherein R² is CH₂CF₃ often do not show peaks corresponding to the CH₂CF₃ protons.

The following Tests demonstrate the control efficacy of compounds of this invention on specific pests. “Control efficacy” represents inhibition of invertebrate pest development (including mortality) that causes significantly reduced feeding. The pest control protection afforded, by the compounds is not limited, however, to these species. See Index Table A for compound, descriptions.

### Biological Examples of the Invention

### Test A

For evaluating control of diamondback moth (Plutella xylostella) the test unit consisted of a small open container with a 12-14-day-old radish plant inside. This was pre-infested with ˜50 neonate larvae that were dispensed into the test unit via corn cob grits using a bazooka inoculator. The larvae moved onto the test plant after being dispensed into the test unit.

Test compounds were formulated using a solution containing 10% acetone, 90% water and 300 ppm X-77® Spreader Lo-Foam Formula non-ionic surfactant containing alkylarylpolyoxyethylene, free fatty acids, glycols and isopropanol (Loveland Industries, Inc. Greeley, Colorado, USA). The formulated compounds were applied in 1 mL of liquid through a SUJ2 atomizer nozzle with ⅛ JJ custom body (Spraying Systems Co. Wheaton, Ill., USA) positioned 1.27 cm (0.5 inches) above the top of each test unit. Test compounds were sprayed at 250 and/or 50 ppm, and replicated three times. After spraying of the formulated test compound, each test unit was allowed to dry for 1 h and then a black, screened cap was placed on top. The test units were held for 6 days in a growth chamber at 25° C. and 70% relative humidity. Plant feeding damage was then visually assessed based on foliage consumed.

Of the compounds of Formula 1 tested at 250 ppm, the following provided very good to excellent levels of control efficacy (40% or less feeding damage and/or 100% mortality): 21, 50, 51, 55, 64, 69, 85, 92 and 93.

Of the compounds of Formula 1 tested at 50 ppm, the following provided very good to excellent levels of control efficacy (40% or less feeding damage and/or 100% mortality): 46, 55, 64, 69, 82, 87, 88, 92, 102, 107, 113, 115, 117, 118 and 119.

### Test B

For evaluating control of corn planthopper (Peregrinus maidis) through contact and/or systemic means, the test unit consisted of a small open container with a 3-4-day-old maize plant (spike) inside. White sand was added to the top of the soil prior to application. Test compounds were formulated and sprayed at 250 and/or 50 ppm, and replicated, three times as described for Test A. After spraying, the test units were allowed to dry for 1 h before they were post-infested with ˜15-20 nymphs (18 to 21 day old) by sprinkling them onto the sand with a salt shaker. A black, screened cap was placed on the top of the cylinder. The test units were held for 6 days in a growth chamber at 22-24° C. and 50-70% relative humidity. Each test unit was then visually assessed for insect mortality.

Of the compounds of Formula 1 tested at 250 ppm, the following provided very good to excellent levels of control efficacy (80% or more mortality): 3, 4, 37, 42, 43, 51, 52, 55, 57, 59, 90, 91 and 93.

Of the compounds of Formula 1 tested at 50 ppm, the following provided very good to excellent levels of control efficacy (80% or more mortality): 37, 42, 43, 55, 59, 87, 88, 92, 102, 105, 107, 113 and 123.

### Test C

For evaluating control of potato leafhopper (Empoasca fabae) through contact and/or systemic means, the test unit consisted of a small open container with a 5-6-day-old Soleil bean plant (primary leaves emerged) inside. White sand was added to the top of the soil and one of the primary leaves was excised prior to application.

Test compounds were formulated and sprayed at 250 and/or 50 ppm, and the tests were replicated three times as described for Test A. After spraying, the test units were allowed to dry for 1 h before they were post-infested with 5 potato leafhoppers (18-21-day-old adults). A black, screened cap was placed on the top of the cylinder. The test units were held for 6 days in a growth chamber at 24° C. and 70% relative humidity. Each test unit was then visually assessed for insect mortality.

Of the compounds of Formula 1 tested at 250 ppm, the following provided very good to excellent levels of control efficacy (80% or more mortality): 3, 43, 50, 55, 85, 90 and 93.

Of the compounds of Formula 1 tested at 50 ppm the following provided very good to excellent levels of control efficacy (80% or more mortality): 43, 85, 87, 88, 92, 93, 102, 106, 107, 113, 119 and 123.

### Test D

For evaluating control of cotton melon aphid (Aphis gossypii) through contact and/or systemic means, the test unit consisted of a small open container with a 6-7-day-old cotton plant inside. This was pre-infested with 30-40 insects on a piece of leaf according to the cut-leaf method described for Test C, and the soil of the test unit was covered with a layer of sand

Test compounds were formulated and sprayed, at 250 and/or 50 ppm as described for Test C. The applications were replicated three times. After spraying, the test units were maintained in a growth chamber and then visually rated as described for Test C.

Of the compounds of Formula 1 tested at 250 ppm, the following resulted in at least 80% mortality: 92, 102 and 105.

Of the compounds of Formula 1 tested at 50 ppm, the following resulted in at least 80% mortality: 92 and 107,

### Test E

For evaluating control of fall armyworm (Spodoptera frugiperda) the test unit consisted of a small open container with a 4-5-day-old maize (corn) plant inside. This was pre-infested (using a core sampler) with 10-15 1-day-old larvae on a piece of insect diet.

Test compounds were formulated and sprayed at 50 ppm as described for Test A. The applications were replicated three times. After spraying, the test units were maintained in a growth chamber at 25° C. and 70% relative humidity and then visually rated, as described for Test A.

Of the compounds of Formula 1 tested the following provided very good to excellent levels of control efficacy (40% or less feeding damage and/or 100% mortality): 92, 113, 117 and 118.

### Test F

For evaluating control of green peach aphid (Myzus persicae) through contact and/or systemic means, the test unit consisted, of a small open container with a 12-15-day-old radish plant inside. This was pre-infested by placing on a leaf of the test plant 30-40 aphids on a piece of leaf excised from a culture plant (cut-leaf method). The aphids moved onto the test plant as the leaf piece desiccated. After pre-infestation, the soil of the test unit was covered with a layer of sand

Test compounds were formulated and sprayed at 250 ppm and/or 50 ppm as described for Test A. The applications were replicated three times. After spraying of the formulated test compound, each test unit was allowed, to dry for 1 h and then a black, screened cap was placed, on top. The test units were held for 6 days in a growth chamber at 19-21° C. and 50-70% relative humidity. Each test unit was then visually assessed for insect mortality.

Of the compounds of Formula 1 tested, at 250 ppm, the following resulted in at least 80% mortality: 87, 88, 92, 102 and 105.

Of the compounds of Formula 1 tested at 50 ppm, the following resulted in at least 80% mortality: 102.

What is claimed is:

1. A compound of Formula 1, an N-oxide or salt thereof, 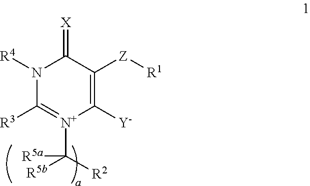
wherein
X is O or S;
Y is O or S;
Z is a direct bond, O, S(O)ₙ, NR⁶, C(R⁷)₂O, OC(R⁷)₂ or EC(═X¹);
X¹ is O, S or NR⁹;
E is O, S or NR^{9a};
R¹ is selected from the group consisting of cyano, CHO, C(═O)OH, C(═O)NH₂ and C(═S)NH₂; C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₅-C₁₀ alkylcycloalkylalkyl and C₃-C₆ cycloalkenyl, each unsubstituted or substituted with at least one substituent independently selected from R³¹; or
R¹ is a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁴;
R² is H, halogen, cyano, hydroxy, amino, nitro, —OCN, —SCN, CHO, C(═O)OH, C(═O)NH₂, C(═S)NH₂, SO₂NH₂, C(═O)R¹⁸, C(═O)OR¹⁸, NHR¹⁸, NR¹⁸R¹⁹, C(═O)NR¹⁸R¹⁹, C(═S)NR¹⁸R¹⁹, SO₂NR¹⁸R¹⁹, OC(═O)R²¹, OC(═O)OR¹⁸, OC(═O)NR¹⁸R¹⁹, N(R¹⁸)C(═O)R²¹, N(R²¹)C(═O)OR¹⁹, N(R²¹)C(═O)NR²¹R²², OSO₂R¹⁸, OSO₂NR²¹R²², NR¹⁸SO₂R¹⁸, NR²¹SO₂NR²¹R²² or Si(R¹⁸R¹⁹R²⁰); or C₁-C₈ alkyl C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl or C₂-C₈ alkynylsulfonyl, each unsubstituted or substituted, with at least one substituent independently selected from halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ and Si(R¹⁰)₃; or a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁵;
R³ is H, C₁-C₆ alkyl, C₁-C₆ haloalkyl, C₂-C₆ alkenyl, C₂-C₆ haloalkenyl, C₂-C₆ alkynyl, C₂-C₆ haloalkynyl or C≡CR²⁵; or
C₃-C₆ cycloalkyl or C₄-C₇ cycloalkylalkyl, each optionally substituted with up to 4 substituents independently selected from the group consisting of halogen, C₁-C₂ alkyl, 1 cyclopropyl and 1 CF₃; or
R³ is phenyl, naphthalenyi or a 5- or 6-membered heteroaromatic ring, each optionally substituted with up to 2 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino and C₂-C₆ dialkylamino;
R⁴ is C₁-C₆ alkyl, C₁-C₆ haloalkyl, C₂-C₆ alkenyl, C₂-C₆ haloalkenyl, C₂-C₆ alkynyl, C₂-C₆ haloalkynyl or C≡CR²⁵; or C₃-C₆ cycloalkyl or C₄-C₇ cycloalkylalkyl, each optionally substituted with up to 4 substituents independently selected from the group consisting of halogen, C₁-C₂ alkyl, 1 cyclopropyl and 1 CF₃; or
R⁴ is phenyl, naphthalenyl or a 5- or 6-membered heteroaromatic ring, each optionally substituted with up to 2 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino and C₂-C₆ dialkylamino; or
R³ and R⁴ are taken together with the contiguous linking nitrogen and carbon atoms to form optionally substituted ring R-1 or ring R-2 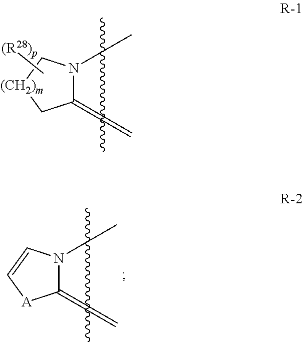
A is C(R^{29a})═C(R^{29b}), S, O or NCH₃, provided that the C(R^{29a})═C(R^{29b}) moiety is oriented so the carbon atom bonded to R^{29b} is connected as R³ in Formula 1;
each R^{5a} and R^{5b} is independently H, halogen, cyano, hydroxy, amino, nitro, —OCN, —SCN, CHO, C(═O)OH, C(═O)NH₂, C(═S)NH₂ or SO₂NH₂; or C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₂ cycloalkylcycloalkyl, C₅-C₈ alkylcycloalkylalkyl, C₃-C₆ cycloalkenyl, C₁-C₆ alkoxy, C₃-C₆ cycloalkoxy, C₄-C₈ cycloalkylalkoxy, C₂-C₆ alkenyloxy or C₂-C₆ alkynyloxy, each optionally substituted with halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ or Si(R¹⁰)₃;
each R⁶, R⁷ and R⁸ is independently H; or C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₆ cycloalkenyl, C₂-C₆ alkylcarbonyl or C₂-C₆ alkoxycarbonyl, each unsubstituted or substituted with at least one substituent independently selected from halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ and Si(R¹⁰)₃;
each R⁹ is independently C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₆ cycloalkenyl, C₂-C₆ alkylcarbonyl or C₂-C₆ alkoxycarbonyl, each unsubstituted or substituted with at least one substituent independently selected from halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ and Si(R¹⁰)₃;
each R^{9a} is independently H; or C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₆ cycloalkenyl, C₂-C₆ alkylcarbonyl or C₂-C₆ alkoxycarbonyl, each unsubstituted or substituted with at least one substituent independently selected from halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³ and Si(R¹⁰)₃;
each R¹⁰, R¹¹, R¹² and R¹³ is independently C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl or C₃-C₆ cycloalkenyl, each unsubstituted or substituted with at least one substituent independently selected from halogen, cyano, nitro, CHO, C(═O)OH, C(═O)NH₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl and C₃-C₆ trialkylsilyl; or phenyl or a 5- or 6-membered heteroaromatic ring, each unsubstituted or substituted with at least one substituent independently selected from C₁-C₆ alkyl, C₁-C₆ haloalkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₆ cycloalkenyl, halogen, cyano, nitro, CHO, C(═O)OH, C(═O)NH₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl and C₃-C₆ trialkylsilyl;
each R¹⁴ is independently halogen, cyano, hydroxy, amino, nitro, SF₅, —OCN, —SCN, CHO, C(═O)OH, C(═O)NH₂, C(═S)NH₂, SO₂NH₂, C(═O)R¹⁸, C(═O)OR¹⁸, NHR¹⁸, NR¹⁸R¹⁹, C(═O)NR¹⁸R¹⁹, C(═S)NR¹⁸R¹⁹, SO₂NR¹⁸R¹⁹, OC(═O)R²¹, OC(═O)OR¹⁸, OC(═O)NR¹⁸R¹⁹, N(R¹⁸)C(═O)R²¹, N(R²¹)C(═O)OR¹⁹, N(R²¹)C(═O)NR²¹R²², OSO₂R¹⁸, OSO₂NR²¹R²², NR¹⁸SO₂R¹⁸, NR²¹SO₂NR²¹R²², Si(R¹⁸R¹⁹R²⁰), C(═NR²¹)R²², C(═NOR²¹)R²², C(═NNR²¹R²²)R²³, C(═NN(C(═O)R¹⁹)R²¹)R²², C(═NN(C(═O)OR¹⁹)R²¹)R²², ON═CR²¹R²², ONR²¹R²², S(═O)(═NR²¹)R²², SO₂NR²¹C(═O)NR²²R²³, P(═X²)R¹⁸R¹⁹, OP(═X²)R¹⁸R¹⁹, OP(═X²)(OR¹⁸)R¹⁹, OP(═X²)(OR¹⁸)OR¹⁹, N═CR²¹R²², NR²¹N═CR²²R²³, NR²¹NR²²R²³, NR²¹C(═X²)NR²²R²³, NR²¹C(═NR²¹)NR²²R²³, NR²¹NR²¹C(═X²)NR²²R²³, NR²¹NR²¹SO₂NR²²R²³, Z¹Q^{t} or Z¹QⁱZ¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl or C₂-C₈ alkynylsulfonyl, each unsubstituted or substituted with at least one substituent independently selected from R¹⁷; or
two R¹⁴ substituents on adjacent ring atoms are taken together with the adjacent ring atoms to form a 5- to 7-membered carbocyclic or heterocyclic ring, each ring containing ring members selected from carbon atoms and up to 3 heteroatoms independently selected from up to 2 O, up to 2 S, and up to 3 N, wherein up to 2 carbon atom ring members are independently selected from C(═O) and C(═S) and the sulfur atom ring members are independently selected from S(═O)ₙ, each ring optionally substituted with up to 3 substituents independently selected from the group consisting of halogen, cyano, hydroxy, amino, nitro, C(═O)OH, C(═O)NH₂, SO₂NH₂, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₃-C₇ cycloalkyl, C₃-C₇ halocycloalkyl, C₄-C₈ alkylcycloalkyl, C₄-C₈ haloalkylcycloalkyl, C₄-C₈ cycloalkylalkyl, C₄-C₈ halocycloalkylalkyl, C₁-C₆ alkoxy, C₁-C₆ haloalkoxy, C₂-C₆ alkoxycarbonyl, C₂-C₆ haloalkoxycarbonyl, C₂-C₆ alkylcarbonyl and C₂-C₆ haloalkylcarbonyl;
each X² is independently O or S;
each Z¹ is independently a direct bond, O, S(O)ₙ, NR⁶, CH(R⁷), C(R⁷)═(R⁷), C≡C, C(R⁷)₂O, OC(R⁷)₂, C(═X¹), C(═X¹)E, EC(═X¹), C(—NOR⁸) or C(═NN(R⁶)₂);
each Qⁱ is independently a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 4 substituents independently selected from the group consisting of halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³, Si(R¹⁰)₃ and R¹⁶;
each Q^{t} is independently a 3- to 10-membered ring or a 7- to 11-membered ring system, each ring or ring system containing ring members selected from carbon atoms and up to 4 heteroatoms independently selected, from up to 2 O, up to 2 S, and up to 4 N, wherein up to 3 carbon atom ring members are independently selected from C(═O) and C(═S) and the sulfur atom ring members are independently selected, from S(═O)ᵤ(═NR²⁴)_{z}, each ring or ring system optionally substituted with up to 5 substituents independently selected from the group consisting of halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³, Si(R¹⁰)₃ and R¹⁶;
each R¹⁵ is independently halogen, cyano, hydroxy, amino, nitro, SF₅, —OCN, —SCN, CHO, C(═O)OH, C(═O)NH₂, C(═S)NH₂, SO₂NH₂, C(═O)R¹⁸, C(═O)OR¹⁸, NHR¹⁸, NR¹⁸R¹⁹, C(═O)NR¹⁸R¹⁹, C(═S)NR¹⁸R¹⁹, SO₂NR¹⁸R¹⁹, OC(═O)R²¹, OC(═O)OR¹⁸, OC(═O)NR¹⁸R¹⁹, N(R¹⁸)C(═O)R²¹, N(R²¹)C(═O)OR¹⁹, N(R²¹)C(═O)NR²¹R²², OSO₂R¹⁸, OSO₂NR²¹R²², NR¹⁸SO₂R¹⁸, NR²¹SO₂NR²¹R²², Si(R¹⁸R¹⁹R²⁰) or Z¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl or C₂-C₈ alkynylsulfonyl, each unsubstituted or substituted with at least one substituent independently selected from R¹⁷; or
two R¹⁵ substituents on adjacent ring atoms are taken together with adjacent ring atoms to form a 5- to 7-membered carbocyclic or heterocyclic ring, each ring containing ring members selected from carbon atoms and up to 3 heteroatoms independently selected from up to 2 O, up to 2 S, and up to 3 N, wherein up to 2 carbon atom ring members are independently selected from C(═O) and C(═S) and the sulfur atom ring members are independently selected from S(═O)ₙ, each ring optionally substituted with up to 3 substituents independently selected from the group consisting of halogen, cyano, hydroxy, amino, nitro, C(═O)OH, C(═O)NH₂, SO₂NH₂, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₃-C₇ cycloalkyl, C₃-C₇ halocycloalkyl, C₄-C₈ alkylcycloalkyl, C₄-C₈ haloalkylcycloalkyl, C₄-C₈ cycloalkylalkyl, C₄-C₈ halocycloalkylalkyl, C₁-C₆ alkoxy, C₁-C₆ haloalkoxy, C₂-C₆ alkoxycarbonyl, C₂-C₆ haloalkoxycarbonyl, C₂-C₆ alkylcarbonyl and C₂-C₆ haloalkylcarbonyl;
each R¹⁶ is independently C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl or C₃-C₆ cycloalkenyl, each unsubstituted or substituted with at least one substituent independently selected from the group consisting of halogen, cyano, nitro, CHO, C(═O)OH, C(═O)NH₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl and C₃-C₆ trialkylsilyl; or phenyl or a 5- or 6-membered heteroaromatic ring, each unsubstituted or substituted with at least one substituent independently selected from the group consisting of C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl, C₃-C₆ cycloalkenyl, halogen, cyano, nitro, CHO, C(═O)OH, C(═O)NH₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl and C₃-C₆ trialkylsilyl;
each R¹⁷ is independently halogen, cyano, nitro, CHO, C(═O)O H, C(═O)NH₂, C(═O)R¹⁰, C(═O)OR¹¹, C(═O)NR¹²R¹³, OR¹¹, S(O)ₙR¹⁰, SO₂NR¹²R¹³, Si(R¹⁰)₃ or Z¹Q^{t};
each R¹⁸, R¹⁹ and R²⁰ is independently C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl or C₃-C₆ cycloalkenyl, each unsubstituted or substituted with at least one substituent independently selected from R¹⁷; or Q^{t};
each R²¹, R²² and R²³ is independently H, C₁-C₆ alkyl, C₂-C₆ alkenyl, C₂-C₆ alkynyl, C₃-C₈ cycloalkyl, C₄-C₈ alkylcycloalkyl, C₄-C₈ cycloalkylalkyl, C₆-C₁₀ cycloalkylcycloalkyl, C₅-C₁₀ alkylcycloalkylalkyl or C₃-C₆ cycloalkenyl, each unsubstituted or substituted with at least one substituent independently selected from R¹⁷; or Q^{t};
each R²⁴ is independently H, cyano, —OCN, —SCN, CHO, C(═O)OH, C(═O)NH₂, C(═S)NH₂, SO₂NH₂, C(═O)R¹⁸, C(═O)OR¹⁸, NHR¹⁸, NR¹⁸R¹⁹, C(═O)NR¹⁸R¹⁹, C(═S)NR¹⁸R¹⁹, SO₂NR¹⁸R¹⁹, OC(═O)R²¹, OC(═O)OR¹⁸, OC(═O)NR¹⁸R¹⁹, N(R¹⁸)C(═O)R²¹, N(R²¹)C(═O)OR¹⁹, N(R²¹)C(═O)NR²¹R²², OSO₂R¹⁸, OSO₂NR²¹R²², NR¹⁸SO₂R¹⁸, NR²¹SO₂NR²¹R²², Si(R¹⁸R¹⁹R²⁰) or Z¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl or C₂-C₈ alkynylsulfonyl, each unsubstituted or substituted with at least one substituent independently selected from R¹⁷;
each R²⁵ is independently Si(R³⁰)₃; or phenyl or pyridinyl, each optionally substituted, with 1 to 3 substituents independently selected, from the group consisting of halogen, cyano, nitro, SF₅, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₂-C₆ alkoxyalkyl, C₁-C₄ alkoxy, C₁-C₄ haloalkoxy, S(O)ₙR²⁶, S(O)₂R²⁷, C₁-C₄ alkylamino and C₂-C₆ dialkylamino;
each R²⁶ is independently C₁-C₄ alkyl or C₁-C₄ haloalkyl;
each R²⁷ is independently C₁-C₄ alkylamino, C₂-C₆ dialkylamino or —NCH₂Z²CH₂;
each Z² is independently CH₂CH₂, CH₂CH₂CH₂ or CH₂OCH₂.
each R²⁸ is independently H, halogen, cyano, CF₃, C₁-C₃ alkyl or C₃-C₆ cycloalkyl;
R^{29a} is H or F;
R^{29b} is H, F, CF₂H or CF₃;
each R³⁰ is independently C₁-C₄ alkyl:
each R³¹ is independently halogen, cyano, nitro, CHO, C(═O)OH, C(═O)NH₂, C(═O)R¹⁰, C(═O)OR¹¹, C(═O)NR¹²R¹³, OR¹¹, S(O)ₙR¹⁰, SO₂NR¹²R¹³, Si(R¹⁰)₃ or Z¹Q^{t};
a is 1, 2 or 3;
m is 0, 1, 2 or 3;
p is 0, 1, 2, 3 or 4;
each n is independently 0, i or 2; and
u and z in each instance of S(═O)ᵤ(═NR²⁴)_{z} are independently 0, 1 or 2, provided that the sum of u and z in each instance of S(═O)ᵤ(═NR²⁴)_{z} is 0, 1 or 2;
provided that when
R¹ is C₁-C₆ alkyl, C₁-C₆ haloalkyl, C₂-C₆ alkenyl, C₂-C₆ haloalkenyl, C₂-C₆ alkynyl, C₂-C₆ haloalkynyl, CR³⁴═C(R³⁴)R²⁵ or C≡CR²⁵; or
C₃-C₇ cycloalkyl, C₄-C₈ cycloalkylalkyl or C₅-C₇ cycloalkenyl, each optionally substituted with 1 to 4 substituents independently selected from the group consisting of halogen, C₁-C₂ alkyl, C₁-C₂ alkoxy, 1 cyclopropyl, 1 CF₃ and 1 OCF₃; or
phenyl, naphthalenyl or a 5- or 6-membered heteroaromatic ring, each optionally substituted with up to 3 substituents independently selected, from the group consisting of halogen, cyano, nitro, C₁-C₄ alkyl, C₂-C₄ alkenyl, C₂-C₄ alkynyl, C₁-C₄ haloalkyl, C₂-C₄ haloalkenyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH2, C₁-C₄ alkoxy, C₁-C₄ haloalkoxy, C₂-C₆ alkoxyalkyl, S(O)ₙR²⁶, S(O)₂R²⁷, C₁-C₄ alkylamino, C₂-C₆ dialkylamino, SF₅, Si(CH₃)₃, CHO, hydroxy, OC(O)R³² and N(R³³)C(O)R³²; or 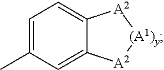
or
CHO; or
an 8- to 10-membered heteroaromatic bicyclic ring system optionally substituted on carbon ring members with up to 3 substituents independently selected from the group consisting of halogen, cyano, nitro, SF₅, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino, C₂-C₆ dialkylamino, Si(CH₃)₃, CHO, hydroxy, OC(O)R³² and N(R³³)C(O)R³², and optionally substituted on nitrogen ring members with methyl; or
phenyl or a 5- or 6-membered heteroaromatic ring, each substituted with GQ¹, each optionally substituted with 1 Q² and each optionally substituted with up to 2 substituents independently selected from the group consisting of halogen, cyano, nitro, SF₅, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino, C₂-C₆ dialkylamino, Si(CH₃)₃, CHO, hydroxy, OC(O)R³² and N(R³³)C(O)R³²; or
phenyl or a 5- or 6-membered heteroaromatic ring, each substituted with LQ¹ and optionally substituted, with up to 2 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino and C₂-C₆ dialkylamino;
each R³² is independently C₁-C₄ alkyl;
each R³³ is independently H or C₁-C₄ alkyl;
each R³⁴ is independently H, F or CH₃;
each A¹ is independently C(R⁵⁰)₂;
each A² is independently C(R⁵¹)₂, O, S(O)ₙ or NR⁵²;
each R⁵⁰ is independently H, F or CH₃;
each R⁵¹ is independently H or C₁-C₄ alkyl;
each R⁵² is independently C₁-C₄ alkyl;
G is a direct bond, O, S(O)ₙ, NH, N(CH₃), CH₂, CH₂O, OCH₂, C(O), C(O)O, OC(O), C(O)NH or NHC(O);
L is a phenyl or 5- or 6-membered heteroaromatic ring optionally substituted with 1 or 2 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino and C₂-C₆ dialkylamino;
Q¹ is phenyl or a 5- or 6-membered heteroaromatic ring, each optionally substituted with 1 to 3 substituents independently selected from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino, C₂-C₆ dialkylamino, SF₅, Si(CH₃)₃, CHO, hydroxy, OC(O)R³² and N(R³³)C(O)R³²;
Q² is phenyl or a 5- or 6-membered heteroaromatic ring, each optionally substituted with up to 2 substituents independently selected, from the group consisting of halogen, cyano, nitro, C₁-C₄ alkyl, C₂-C₄ alkenyl, C₂-C₄ alkynyl, C₁-C₄ haloalkyl, C₂-C₄ alkylcarbonyl, C₂-C₄ haloalkylcarbonyl, C₂-C₄ alkoxycarbonyl, C₂-C₄ alkylaminocarbonyl, C₃-C₇ dialkylaminocarbonyl, C(O)NCH₂Z²CH₂, C₁-C₄ alkoxy, C₁-C₄ haloalkoxy, C₂-C₆ alkoxyalkyl, S(O)ₙR²⁶, S(O)₂R²⁷, C₁-C₄ alkylamino and C₂-C₆ dialkylamino;
a is 1;
y is 1 or 2;
R^{5a} is H, halogen, cyano or C₁-C₄ alkyl;
R^{5b} is H, halogen or CH₃;
R² is C₁-C₅ alkyl, C₁-C₅ haloalkyl, C₂-C₅ alkenyl, C₂-C₅ haloalkenyl, C₂-C₅ alkynyl, C₂-C₅ haloalkynyl, CO₂R³³, CH₂OR³³, CH₂CH₂OR³³, CH₂S(O)ₙR³³ or CH₂CH₂S(O)ₙR³³; or
C₃-C₆ cycloalkyl or C₄-C₆ cycloalkylalkyl, each optionally substituted with up to 4 substituents selected from the group consisting of halogen, C₁-C₂ alkyl, 1 cyclopropyl and 1 CF₃; or
an optionally substituted 5- or 6-membered heterocyclic ring;
then Z is O, S(O)ₙ, NR⁶, C(R⁷)₂O, OC(R⁷)₂ or EC(═X¹).

2. A compound of claim 1 wherein
A is C(R^{29a})═C(R^{29b}), S or NCH₃.
X is O;
Y is O;
Z is a direct bond, O or NR⁶;
R¹ is C₁-C₈ alkyl optionally substituted with halogen; or a 3- to 10-membered ring or a 7- to 11-membered ring system, each ring or ring system containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁴;
R² is C(═O)OR¹⁸; or C₁-C₈ alkyl optionally substituted with halogen; or a 3- to 10-membered ring or a 7- to 11-membered ring system, each ring or ring system containing ring members selected, from carbon atoms and up to 4 heteroatoms independently selected from up to 2 O, up to 2 S, and up to 4 N, wherein up to 3-carbon atom ring members are independently selected from C(═O) and C(═S) and the sulfur atom ring members are independently selected from S(═O)ᵤ(═NR²⁴)_{z}, each ring or ring system optionally substituted with up to 5 substituents independently selected from R¹⁵;
R³ is H, C₁-C₆ alkyl, C₁-C₆ haloalkyl, C₂-C₆ alkenyl, C₂-C₆ haloalkenyl, C₂-C₆ alkynyl, C₂-C₆ haloalkynyl or C≡CR²⁵; or C₃-C₆ cycloalkyl or C₄-C₇ cycloalkylalkyl, each optionally substituted with up to 4 substituents independently selected from the group consisting of halogen, C₁-C₂ alkyl, 1 cyclopropyl and 1 CF₃;
R⁴ is C₁-C₆ alkyl, C₁-C₆ haloalkyl, C₂-C₆ alkenyl, C₂-C₆ haloalkenyl, C₂-C₆ alkynyl, C₂-C₆ haloalkynyl or C≡CR²⁵; or C₃-C₆ cycloalkyl or C₄-C₇ cycloalkylalkyl, each optionally substituted with up to 4 substituents independently selected, from the group consisting of halogen, C₁-C₂ alkyl, 1 cyclopropyl and 1 CF₃; or
R³ and R⁴ are taken together with the contiguous linking nitrogen and carbon atoms to form an optionally substituted ring R-1 or ring R-2;
R^{5a} and R^{5b} are H; and
a is 1.

3. A compound of claim 2 wherein
A is C(R^{29a})═C(R^{29b}) or NCH₃;
Z is a direct bond;
R¹ is a phenyl ring, a 5- or 6-membered heteroaromatic ring, or a 5- or 6-membered non-aromatic ring optionally substituted with up to 3 substituents independently selected from R¹⁴;
R² is pyridinyl, pyrimidinyl or thiazolyl, each optionally substituted with up to 3 substituents independently selected from R¹⁵;
R³ is C₁-C₆ alkyl, C₁-C₆ haloalkyl or cyclopropyl;
R⁴ is C₁-C₆ alkyl, C₁-C₆ haloalkyl or cyclopropyl; or
R³ and R⁴ are taken together with the contiguous linking nitrogen and carbon atoms to form the optionally substituted ring R-1 or ring R-2;
each R¹⁴ is independently halogen, cyano, SF₅, CHO, C(═O)R¹⁸, C(═O)OR¹⁸, C(═O)NR¹⁸R¹⁹, C(═NOR²¹)R²², Z¹Q^{t} or Z¹QⁱZ¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio or C₂-C₈ alkynylthio, each unsubstituted or substituted with at least one substituent independently selected, from R¹⁷;
each R¹⁵ is independently halogen, cyano, SF₅, CHO, C(═O)R¹⁸, C(═O)OR¹⁸, C(═O)NR¹⁸R¹⁹, Z¹Q^{t}; or C₁-C₈ alkyl, C₂-C₈ alkenyl, C₂-C₈ alkynyl, C₃-C₁₀ cycloalkyl, C₄-C₁₀ alkylcycloalkyl, C₄-C₁₀ cycloalkylalkyl, C₁-C₈ alkoxy, C₃-C₈ cycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₄-C₁₀ cycloalkylalkylthio, C₂-C₈ alkenylthio and C₂-C₈ alkynylthio, each unsubstituted or substituted with at least one substituent independently selected from R¹⁷;
R¹⁷ is halogen, OR¹¹ or Z¹Q^{t};
R²⁸ is CH₃; and
Z¹ is a direct bond;
each Qⁱ and Q^{t} is independently a 5- to 6-membered ring, each ring containing ring members selected from carbon atoms and up to 4 heteroatoms independently selected from up to 2 O, up to 2 S, and up to 4 N, wherein up to 3 carbon atom ring members are independently selected from C(═O) and C(═S) and the sulfur atom ring members are independently selected from S(═O)ᵤ(═NR²⁴)_{z}, each ring optionally substituted with up to 5 substituents independently selected from the group consisting of halogen, cyano, OR¹¹, S(O)ₙR¹⁰ and R¹⁶;
m is 2; and
p is 1.

4. A compound of claim 3 wherein
R¹ is phenyl optionally substituted with up to 3 substituents independently selected from R¹⁴;
R³ and R⁴ are taken together with the contiguous linking nitrogen and carbon atoms to
form the optionally substituted ring R-2; and
each Qⁱ and Q^{t} is independently phenyl or pyridinyl, each optionally substituted with up to 5 substituents independently selected from the group consisting of halogen and C₁-C₄ haloalkyl.

5. A compound of claim 4 wherein
A is C(R^{29a})═C(R^{29b});
each R¹⁴ is independently halogen, cyano, C(═O)OR¹⁸, C(═O)NR¹⁸R¹⁹, C(═NOR²¹)R²², Z¹Q^{t}; or C₁-C₈ alkyl, C₁-C₈ alkoxy or C₁-C₈ alkylthio, each optionally substituted with halogen;
each R¹⁵ is independently halogen or C₁-C₄ alkyl;
R^{29a} is H; and
R^{29b} is H or F.

6. A compound selected from the group consisting of
1-[(5-chloro-2-thienyl)methyl]-2-hydroxy-4-oxo-3-phenyl-4H-pyrido[1,2-a]pyrimidinium inner salt;
2-hydroxy-4-oxo-3-phenoxy-1-propyl-4H-pyrido[1,2-a]pyrimidinium inner salt;
1-[(4-chlorophenyl)methyl]-2-hydroxy-4-oxo-3-[3-(trifluoromethoxy)phenyl]-4H-pyrido[1,2-a]pyrimidinium inner salt;
1-[(6-chloro-3-pyridinyl)methyl]-2-hydroxy-4-oxo-3-[3-[[(2,2,2-trifluoroethyl)amino]carbonyl]phenyl]-4H-pyrido[1,2-a]pyrimidinium inner salt;
2-hydroxy-4-oxo-1-[2-(3-pyridinyl)ethyl]-3-[3-(trifluoromethoxy)phenyl]-4H-pyrido[1,2-a]pyrimidinium inner salt;
3-(4-fluorophenyl)-2-hydroxy-4-oxo-1-[2-(2-pyridinyl)ethyl]-4H-pyrido[1,2-a]pyrimidinium inner salt;
1-[(6-chloro-3-pyridinyl)methyl]-3-(2,3-dihydro-1H-inden-1-yl)-2-hydroxy-4-oxo-4H-pyrido-[1,2-a]pyrimidinium inner salt;
1-[(2-chloro-5-thiazolyl)methyl]-3-(2,3-dihydro-1H-inden-1-yl)-2-hydroxy-4-oxo-4H-pyrido[1,2-a]pyrimidinium inner salt;
2-hydroxy-1-methyl-4-oxo-3-phenyl-4H-pyrido[1,2-a]pyrimidinium inner salt;
1-[2-(acetylamino)ethyl]-2-hydroxy-4-oxo-3-phenyl-4H-pyrido[1,2-a]pyrimidinium inner salt;
3-[3-(cyanomethoxy)phenyl]-2-hydroxy-4-oxo-1-(2,2,2-trifluoroethyl)-4H-pyrido[1,2-a]pyrimidinium inner salt;
1-[(6-chloro-3 pyridinyl)methyl]-3-[3-(cyanomethoxy)phenyl]-2-hydroxy-4-oxo-4H-pyrido[1,2-a]pyrimidinium inner salt;
3-[2-[(6-chloro-3-pyridinyl)methoxy]-4-fluorophenyl]-2-hydroxy-4-oxo-1-(2,2,2-trifluoroethyl)-4H-pyrido[1,2-a]pyrimidinium inner salt;
3-[3-[(6-chloro-3-pyridinyl)methoxy]phenyl]-2-hydroxy-4-oxo-1-(2,2,2)-trifluoroethoxy)phenyl]-4H-pyrido[1,2-a]pyrimidinium inner salt;
3-[3-[(6-chloro-3-pyridinyl)methoxy]phenyl]-1-[(6-chloro-3-pyridinyl)methyl]-2-hydroxy-4-oxo-4H-pyrido[1,2-a]pyrimidinium inner salt;
1-[(6-chloro-3-pyridinyl)methyl]-2-hydroxy-4-oxo-3-[3-(2,2,2-trifluoroethoxy)phenyl]4H-pyrido[1,2-a]pyrimidinium inner salt;
1-[(6-chloro-3-pyridinyl)methyl]-3-[4-fluoro-3-(2-pyridinylmethoxy)phenyl]-2-hydroxy-4-oxo-4H-pyrido[1,2-a]pyrimidinium inner salt;
3-[3-[(6-chloro-2-pyridinyl)methoxy]phenyl]-2-hydroxy-4-oxo-1-(2,2,2-trifluoroethyl)-4H-pyrido[1,2-a]pyrimidinium inner salt;
2-hydroxy-1-[(4-methoxyphenyl)methyl]-4-oxo-3-phenyl-4H-pyrido[1,2-a]pyrimidinium inner salt;
3-[3-(3-bromo-4,5-dihydro-5-isoxazolyl)phenyl]-1-[(2-chloro-5-thiazolyl)methyl]-2-hydroxy-4-oxo-4H-pyrido[1,2-a]pyrimidinium inner salt;
1-[(2-chloro-5-thiazolyl)methyl]-3-[3-(4,5-dihydro-3-methyl-5-isoxazolyl)phenyl]-2-hydroxy-4-oxo-4H-pyrido[1,2-a]pyrimidinium inner salt;
1-(1,3-dioxolan-2-ylmethyl)-2-hydroxy-4-oxo-3-phenyl-4H-pyrido[1,2-a]pyrimidinium inner salt;
1-(1,4-dioxan-2-ylmethyl)-2-hydroxy-4-oxo-3-phenyl-4H-pyrido[1,2-a]pyrimidinium inner salt; and
1-[(2-chloro-5-thiazolyl)methyl]-2-hydroxy-3-[3-[1-(methoxyimino)ethyl]phenyl]-4-oxo-4H-pyrido[1,2-a]pyrimidinium inner salt.

7. A composition comprising a compound of claim 1 and at least one additional component selected from the group consisting of surfactants, solid diluents and liquid diluents.

8. The composition of claim 7 further comprising at least one additional biologically active compound or agent.

9. The composition of claim 8 wherein the at least one additional biologically active compound or agent is selected from the group consisting of abamectin, acephate, acequinocyl, acetamiprid, acrinathrin, amidoflumet, amitraz, avermectin, azadirachtin, azinphos-methyl, bensultap, bifenthrin, bifenazate, bistrifluoron, borate, 3-bromo-1-(3-chloro-2-pyridinyl)-N-[4-cyano-2-methyl-6-[(methylamino)carbonyl]phenyl]-1H-pyrazole-5-carboxamide (cyantraniliprole), buprofezin, cadusafos, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazoie, fenbutatin oxide, fenothiocarb, fenoxycarb, fenpropathrin, fenvalerate, fipronil, flonicamid, flubendiamide, flucythrinate, flufenerim, flufenoxuron, fluvalinate, tau-fluvalinate, fonophos, formetanate, fosthiazate, halofenozide, hexaflumuron, hexythiazox, hydramethylnon, imidacloprid, indoxacarb, insecticidal soaps, isofenphos, lufenuron, malathion, metaflumizone, metaldehyde, methamidophos, methidathion, methiodicarb, methomyl, methoprene, methoxychlor, metofluthrin, monocrotophos, methoxyfenozide, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, spinetoram, spinosad, spirodiclofen, spiromesifen, spirotetramat, sulprofos, sulfoxaflor, tebufenozide, tebufenpyrad, teflubenzuron, tefluthrin, terbufos, tetrachlorvinphos, tetramethrin, thiacloprid, thiamethoxam, thiodicarb, thiosultap-sodium, tolfenpyrad, tralomethrin, triazamate, trichlorfon, triflumuron, Bacillus thuringiensis delta-endotoxins, entoniopathogenic bacteria, entomopathogenic viruses and entomopathogenic fungi.

10. A composition for protecting an animal from an invertebrate parasitic pest comprising a parasiticidally effective amount of a compound of claim 1 and at least one carrier.

11. A method for controlling an invertebrate pest comprising contacting the invertebrate pest or its environment with a biologically effective amount of a compound, of claim 1.

12. A treated seed comprising a compound of claim 1 in an amount of from about 0.0001 to 1% by weight of the seed before treatment.
